(19) United States
(12) Patent Application Publication
(10) Pub. No.: US 2008/0312206 A1
(43) Pub. Date: Dec. 18, 2008

(54) Chemical Compounds-149

(75) Inventors: Brian Aquila, Marlborough, MA (US); Donald Cook, Derry, NH (US); Leslie Dakin, Natick, MA (US); Stephanos Ioannidis, Watertown, MA (US); Paul Lyne, Arlington, MA (US); David Scott, Somerville, MA (US); Xiaolan Zheng, Waltham, MA (US)

Correspondence Address: ASTRAZENECA R&D BOSTON, 35 GATEHOUSE DRIVE, WALTHAM, MA, 02451-1215, US

(73) Assignee: AstraZeneca AB, Sodertalje (SE)

(21) Appl. No.: 12/097,652

(22) PCT Filed: Dec. 19, 2006
(86) PCT No.: PCT/GB06/04743

§ 371 Date: Jun. 16, 2008

Related U.S. Application Data
(60) Provisional application No. 60/753,299, filed on Dec. 22, 2005.

(51) Int. Cl. A61K 31/427; C07D 277/46; A61K 31/426; A61P 35/00; C07D 417/02

(52) U.S. Cl. 514/210.2; 548/195; 514/371

(57) ABSTRACT

The invention relates to chemical compounds, or pharmaceutically acceptable salts thereof of the formula which possess CSF 1R kinase inhibitory activity and are accordingly useful for their anti cancer activity and thus in methods of treatment of the human or animal body. The invention also relates to processes for the manufacture of said chemical compounds, to pharmaceutical compositions containing them and to their use in the manufacture of medicaments of use in the production of an anti-cancer effect in a warm blooded animal such as man.

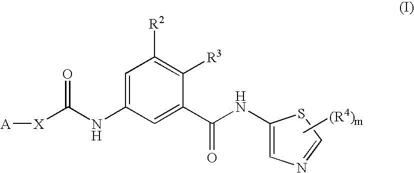

### BACKGROUND OF THE INVENTION

The invention relates to chemical compounds, or pharmaceutically acceptable salts thereof which possess colony stimulating factor 1 receptor (CSF-1R) kinase inhibitory activity and are accordingly useful for their anti-cancer activity and thus in methods of treatment of the human or animal body. The invention also relates to processes for the manufacture of said chemical compounds, to pharmaceutical compositions containing them and to their use in the manufacture of medicaments of use in the production of an anti-cancer effect in a warm-blooded animal such as man.

Receptor tyrosine kinases (RTK's) are a sub-family of protein kinases that play a critical role in cell signalling and are involved in a variety of cancer related processes including cell proliferation, survival, angiogenesis, invasion and metastasis. There are believed to be at least 96 different RTK's including CSF-1R.

CSF-1R or c-fms was originally identified as the oncogene v-fms from the feline sarcoma virus. CSF-1R is a member of the class III RTK's along with c-Kit, fms-related tyrosine kinase 3 (Flt3) and Platelet-derived growth factor receptor α and β (PDGFRα and PDGFRβ). All of these kinases have been implicated in the process of tumorigenesis. CSF-1R is normally expressed as an immature 130 kDa transmembrane protein and ultimately results in a mature 145-160 kDa cell surface N-linked glycosylated protein. Macrophage colony stimulating factor (M-CSF or CSF-1), the ligand for CSF-1R, binds to the receptor resulting in dimerization, auto-phosphorylation of the receptor and subsequent activation of downstream signal transduction cascades (C. J. Sherr, Biochim Biophys Acta, 1988, 948: 225-243).

CSF-1R is normally expressed in myeloid cells of the mononuclear phagocytic lineage and their bone-marrow progenitors as well as the epithelial cells of the ducts and alveoli in the lactating, but not normal resting, breast tissue. CSF-1R activation stimulates the proliferation, survival, motility and differentiation of cells of the monocyte/macrophage lineage. The mature macrophage plays a key role in normal tissue development and immune defence (F. L. Pixley and E. R. Stanley, Trends in Cell Biology, 2004, 14(11): 628-638). For example, osteoblasts secrete CSF-1 and activate the receptor on osteoclastic progenitors resulting in differentiation into mature osteoclasts (S. L. Teitelbaum, Science, 2000, 289: 1504-1508). The CSF-1R axis plays an important role in placental development, embryonic implantation, mammary gland ductal and lobuloalveolar development and lactation (E. Sapi, Exp Biol Med, 2004, 229:1-11).

Transfection of CSF-1R with or without CSF-1 induces transformation and in vivo tumorigenicity of NIH3T3 (Rat2 and ovarian granulosa cells. Autocrine and/or paracrine signaling mechanisms have been implicated in the activation of CSF-1R in the tumour epithelium and tumour associated macrophage. Aberrant expression and activation of CSF-1R and/or its ligand have been found in human myeloid leukaemia, prostate, breast, ovarian, endometrial and a variety of other cancers. A number of studies have demonstrated that the overexpression of CSF-1R is associated with poor prognosis in several of these cancers. In addition, the CSF-1/CSF-1R axis plays a key role in the regulation of tumour-associated macrophage, which have been postulated to play a significant role in tumour angiogenesis, invasion and progression (E. Sapi, Exp Biol Med, 2004, 229: 1-11).

### SUMMARY OF THE INVENTION

Accordingly, the present invention provides a compound of formula (I):

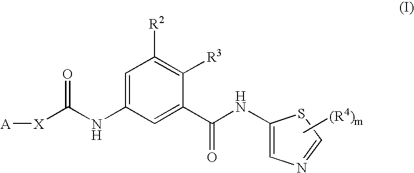

or a pharmaceutically acceptable salt thereof; wherein:

A is

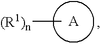

wherein Ring A is aryl, heteroaryl, carbocyclyl or heterocyclyl; wherein if said heterocyclyl contains an —NH— moiety that nitrogen may be optionally substituted by a group selected from R⁵; or

A is C₁₋₆alkyl, C₂₋₆alkenyl, or C₂₋₆alkynyl, each of which may be optionally substituted with 1, 2, or 3 substituents selected from aryl, heteroaryl, halo, nitro, cyano, hydroxy, trifluoromethoxy, amino, carboxy, carbamoyl, mercapto, sulphamoyl, C₁₋₆alkoxy, C₁₋₆alkanoyl, C₁₋₆alkanoyloxy, N—(C₁₋₆alkyl)amino, N,N—(C₁₋₆alkyl)₂-amino, C₁₋₆alkanoylamino, N—(C₁₋₆alkyl)carbamoyl, N,N—(C₁₋₆alkyl)₂-carbamoyl, C₁₋₆allylS(O)ₐ wherein a is 0 to 2, C₁₋₆alkoxycarbonyl, C₁₋₆alkoxycarbonylamino, N—(C₁₋₆alkyl)sulphamoyl, N,N—(C₁₋₆alkyl)₂sulphamoyl, N—(C₁₋₆alkyl)-N—(C₁₋₆alkoxy)sulphamoyl, N,N′—(C₁₋₆alkyl)₂ureido, N′,N—(C₁₋₆alkyl)₂ureido, N—(C₁₋₆alkyl)-N′,N′—(C₁₋₆alkyl)₂ureido, C₁₋₆alkylsulphonylamino, carbocyclyl-R⁶— or heterocyclyl-R⁷—; wherein A may be optionally substituted on carbon by one or more R⁸; and wherein if said heterocyclyl contains an —NH— moiety that nitrogen may be optionally substituted by a group selected from R⁹;

R¹ is a substituent on carbon and is selected from halo, nitro, cyano, hydroxy, trifluoromethoxy, amino, carboxy, carbamoyl, mercapto, sulphamoyl, C₁₋₆alkyl, C₂₋₆alkenyl, C₂₋₆alkynyl, C₁₋₆alkoxy, C₁₋₆alkanoyl, C₁₋₆alkanoyloxy, N—(C₁₋₆alkyl)amino, N,N—(C₁₋₆alkyl)₂-amino, C₁₋₆alkanoylamino, N—(C₁₋₆alkyl)carbamoyl, N,N—(C₁₋₆alkyl)₂-carbamoyl, C₁₋₆alkylS(O)ₐ wherein a is 0 to 2, C₁₋₆alkoxycarbonyl, C₁₋₆alkoxycarbonylamino, N—(C₁₋₆alkyl)sulphamoyl, N,N—(C₁₋₆alkyl)₂sulphamoyl, N—(C₁₋₆alkyl)-N—(C₁₋₆alkoxy)sulphamoyl, N,N′—(C₁₋₆alkyl)₂ureido, N′,N′—(C₁₋₆alkyl)₂ureido, N—(C₁₋₆alkyl)-N′,N′—(C₁₋₆alkyl)₂ureido, C₁₋₆alkylsulphonylamino, carbocyclyl-R⁶— or heterocyclyl-R⁷—; wherein R¹ may be optionally substituted on carbon by one or more R⁸; and wherein if said heterocyclyl contains an —NH— moiety that nitrogen may be optionally substituted by a group selected from R⁹;

n is selected from 0-4; wherein the values of R¹ may be the same or different;

X is absent or is O or NRₐ, wherein Rₐ is H or C₁₋₆alkyl;

R² and R³ are independently selected from hydrogen, halo, nitro, cyano, hydroxy, trifluoromethoxy, amino, carboxy, carbamoyl, mercapto, sulphamoyl, C₁₋₆alkyl, C₂₋₆alkenyl, C₂₋₆alkynyl, C₁₋₆alkoxy, C₁₋₆alkanoyl, C₁₋₆alkanoyloxy, N—(C₁₋₆alkyl)amino, N,N—(C₁₋₆alkyl)₂-amino, C₁₋₆alkanoylamino, N—(C₁₋₆alkyl)carbamoyl, N,N—(C₁₋₆alkyl)₂-carbamoyl, C₁₋₆alkylS(O)ₐ wherein a is 0 to 2, C₁₋₆alkoxycarbonyl, N—(C₁₋₆alkyl)sulphamoyl, N,N—(C₁₋₆alkyl)₂sulphamoyl, C₁₋₆alkylsulphonylamino, carbocyclyl-R¹⁰— or heterocyclyl-R¹¹—; wherein R² may be optionally substituted on carbon by one or more R²; and wherein if said heterocyclyl contains an —NH— moiety that nitrogen may be optionally substituted by a group selected from R¹³;

R⁴ is selected from halo, cyano, amino, carboxy, carbamoyl, mercapto, sulphamoyl, ureido, C₁₋₆alkyl, C₂₋₆alkenyl, C₂₋₆alkynyl, C₁₋₆alkoxy, C₁₋₆alkanoyl, C₁₋₆alkanoyloxy, N—(C₁₋₆alkyl)amino, N,N—(C₁₋₆alkyl)₂-amino, C₁₋₆alkanoylamino, N—(C₁₋₆alkyl)carbamoyl, N,N—(C₁₋₆alkyl)₂-carbamoyl, C₁₋₆alkylS(O)ₐ wherein a is 0 to 2, C₁₋₆alkoxycarbonyl, N—(C₁₋₆alkyl)sulphamoyl, N,N—(C₁₋₆alkyl)₂sulphamoyl, C₁₋₆alkylsulphonylamino, carbocyclyl-R¹⁴— or heterocyclyl-R¹⁵—; wherein R⁴ may be optionally substituted on carbon by one or more R¹⁶; and wherein if said heterocyclyl contains an —NH— moiety that nitrogen may be optionally substituted by a group selected from R¹⁷;

m is selected from 0-2; wherein the values of R⁴ may be the same or different;

R⁸ and R¹² are independently selected from halo, nitro, cyano, hydroxy, trifluoromethoxy, amino, carboxy, carbamoyl, mercapto, sulphamoyl, C₁₋₆alkyl, C₂₋₆alkenyl, C₂₋₆alkynyl, C₁₋₆alkoxy, C₁₋₆alkanoyl, C₁₋₆alkanoyloxy, N—(C₁₋₆alkyl)amino, N,N—(C₁₋₆alkyl)₂-amino, N—(C₁₋₆alkyl)-N—(C₁₋₆alkoxy)amino, C₁₋₆alkanoylamino, N—(C₁₋₆alkyl)carbamoyl, N,N—(C₁₋₆alkyl)₂-carbamoyl, C₁₋₆alkylS(O)ₐ wherein a is 0 to 2, C₁₋₆alkoxycarbonyl, N—(C₁₋₆alkyl)sulphamoyl, N,N—(C₁₋₆alkyl)₂sulphamoyl, C₁₋₆alkylsulphonylamino, carbocyclyl-R¹⁸— or heterocyclyl-R¹⁹—; wherein R⁸ and R¹² independently of each other may be optionally substituted on carbon by one or more R²⁰; and wherein if said heterocyclyl contains an —NH— moiety that nitrogen may be optionally substituted by a group selected from R²¹;

R¹⁶ is selected from halo, nitro, cyano, hydroxy, trifluoromethoxy, amino, carboxy, carbamoyl, mercapto, sulphamoyl, C₁₋₆alkyl, C₂₋₆alkenyl, C₂₋₆alkynyl, C₁₋₆alkoxy, C₁₋₆alkanoyl, C₁₋₆alkanoyloxy, N—(C₁₋₆alkyl)amino, N,N—(C₁₋₆alkyl)₂-amino, C₁₋₆alkanoylamino, N—(C₁₋₆alkyl)carbamoyl, N,N—(C₁₋₆alkyl)₂-carbamoyl, C₁₋₆alkylS(O)ₐ wherein a is 0 to 2, C₁₋₆alkoxycarbonyl, N—(C₁₋₆alkyl)sulphamoyl, N,N—(C₁₋₆alkyl)₂sulphamoyl, C₁₋₆alkylsulphonylamino, carbocyclyl-R²²— or heterocyclyl-R²³—; wherein R¹⁶ may be optionally substituted on carbon by one or more R²⁴; and wherein if said heterocyclyl contains an —NH— moiety that nitrogen may be optionally substituted by a group selected from R²⁵;

R⁶, R⁷, R¹⁰, R¹¹, R¹⁴, R¹⁵, R¹⁸, R¹⁹, R²² and R²³ are independently selected from a direct bond, —O—, —N(R²⁶)—, —C(O)—, —N(R²⁷)C(O)—, —C(O)N(R²⁸)—, —S(O)ₛ—, —SO₂N(R²⁹)— or —N(R³⁰)SO₂—; wherein R²⁶, R²⁷, R²⁸, R²⁹ and R³⁰ are independently selected from hydrogen or C₁₋₆alkyl and s is 0-2;

R⁵, R⁹, R¹³, R¹⁷, R²¹ and R²⁵ are independently selected from C₁₋₆alkyl, C₁₋₆alkanoyl, C₁₋₆alkylsulphonyl, C₁₋₆alkoxycarbonyl, carbamoyl, N—(C₁₋₆alkyl)carbamoyl, N,N—(C₁₋₆alkyl)carbamoyl, benzyl, benzyloxycarbonyl, benzoyl and phenylsulphonyl;

R²⁰ and R²⁴ are independently selected from halo, nitro, cyano, hydroxy, trifluoromethoxy, trifluoromethyl, amino, carboxy, carbamoyl, mercapto, sulphamoyl, methyl, ethyl, methoxy, ethoxy, acetyl, acetoxy, methylamino, ethylamino, dimethylamino, diethylamino, N-methyl-N-ethylamino, acetylamino, N-methylcarbamoyl, N-ethylcarbamoyl, N,N-dimethylcarbamoyl, N,N-diethylcarbamoyl, N-methyl-N-ethylcarbamoyl, phenyl, methylthio, ethylthio, methylsulphinyl, ethylsulphinyl, mesyl, ethylsulphonyl, methoxycarbonyl, ethoxycarbonyl, N-methylsulphamoyl, N-ethylsulphamoyl, N,N-dimethylsulphamoyl, N,N-diethylsulphamoyl or N-methyl-N-ethylsulphamoyl.

In another aspect, the invention relates to compounds of formula (I), wherein:

A is

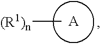

wherein Ring A is aryl, heteroaryl, carbocyclyl or heterocyclyl; wherein if said heteroaryl or heterocyclyl contains an —NH— moiety that nitrogen may be optionally substituted by a group selected from R⁵; or

A is C₁₋₆alkyl, C₂₋₆alkenyl, or C₂₋₆alkynyl; wherein A may be optionally substituted on carbon by one or more R^{8a}; and wherein if said heterocyclyl contains an —NH— moiety that nitrogen may be optionally substituted by a group selected from R^{9a};

R¹ is a substituent on carbon and is selected from halo, nitro, cyano, hydroxy, trifluoromethoxy, amino, carboxy, carbamoyl, mercapto, sulphamoyl, C₁₋₆alkyl, C₂₋₆alkenyl, C₂₋₆alkynyl, C₁₋₆alkoxy, C₁₋₆alkanoyl, C₁₋₆alkanoyloxy, N—(C₁₋₆alkyl)amino, N,N—(C₁₋₆alkyl)₂-amino, C₁₋₆alkanoylamino, N—(C₁₋₆alkyl)carbamoyl, N,N—(C₁₋₆alkyl)₂-carbamoyl, C₁₋₆alkylS(O)ₐ wherein a is 0 to 2, C₁₋₆alkoxycarbonyl, C₁₋₆alkoxycarbonylamino, N—(C₁₋₆alkyl)sulphamoyl, N,N—(C₁₋₆alkyl)₂sulphamoyl, N—(C₁₋₆alkyl)-N—(C₁₋₆alkoxy)sulphamoyl, N,N′—(C₁₋₆alkyl)₂ureido, N′,N′—(C₁₋₆alkyl)₂ureido, N—(C₁₋₆alkyl)-N′,N′—(C₁₋₆alkyl)₂ureido, C₆alkylsulphonylamino, carbocyclyl-R⁶— or heterocyclyl-R⁷—; wherein R¹ may be optionally substituted on carbon by one or more R⁸; and wherein if said heterocyclyl contains an —NH— moiety that nitrogen may be optionally substituted by a group selected from R⁹;

n is selected from 0-4; wherein the values of R¹ may be the same or different;

X is absent or is O or NRₐ, wherein Rₐ is H or C₁₋₆alkyl;

R² and R³ are independently selected from hydrogen, halo, nitro, cyano, hydroxy, trifluoromethoxy, amino, carboxy, carbamoyl, mercapto, sulphamoyl, C₁₋₆alkyl, C₂₋₆alkenyl, C₂₋₆alkynyl, C₁₋₆alkoxy, C₁₋₆alkanoyl, C₁₋₆alkanoyloxy, N—(C₁₋₆alkyl)amino, N,N—(C₁₋₆alkyl)₂-amino, C₁₋₆alkanoylamino, N—(C₁₋₆alkyl)carbamoyl, N,N—(C₁₋₆alkyl)₂-carbamoyl, C₁₋₆alkylS(O)ₐ wherein a is 0 to 2, C₁₋₆alkoxycarbonyl, N—(C₁₋₆alkyl)sulphamoyl, N,N—(C₁₋₆alkyl)₂sulphamoyl, C₁₋₆alkylsulphonylamino, carbocyclyl-R¹⁰— or heterocyclyl-R¹¹; wherein R² may be optionally substituted on carbon by one or more R¹²; and wherein if said heterocyclyl contains an —NH— moiety that nitrogen may be optionally substituted by a group selected from R¹³;

R⁴ is selected from halo, cyano, amino, carboxy, carbamoyl, mercapto, sulphamoyl, ureido, C₁₋₆alkyl, C₂₋₆alkenyl, C₂₋₆alkynyl, C₁₋₆alkoxy, C₁₋₆alkanoyl, C₁₋₆alkanoyloxy, N—(C₁₋₆alkyl)amino, N,N—(C₁₋₆alkyl)₂-amino, C₁₋₆alkanoylamino, N—(C₁₋₆alkyl)carbamoyl, N,N—(C₁₋₆alkyl)₂-carbamoyl, N—(C₁₋₆alkyl)-N—(C₁₋₆alkoxy)carbamoyl, C₁₋₆alkylS(O)ₐ wherein a is 0 to 2, C₁₋₆alkoxycarbonyl, N—(C₁₋₆alkyl)sulphamoyl, N,N—(C₁₋₆alkyl)₂sulphamoyl, C₁₋₆alkylsulphonylamino, carbocyclyl-R¹⁴— or heterocyclyl-R¹⁵—; wherein R⁴ may be optionally substituted on carbon by one or more R¹⁶; and wherein if said heterocyclyl contains an —NH— moiety that nitrogen may be optionally substituted by a group selected from R¹⁷;

m is selected from 0-2; wherein the values of R⁴ may be the same or different;

R⁸, R^{8a}, and R¹² in each occurrence are independently selected from aryl, heteroaryl, halo, nitro, cyano, hydroxy, trifluoromethoxy, amino, carboxy, carbamoyl, mercapto, sulphamoyl, C₁₋₆alkyl, C₂₋₆alkenyl, C₂₋₆alkynyl, C₁₋₆alkoxy, C₁₋₆alkanoyl, C₁₋₆alkanoyloxy, N—(C₁₋₆alkyl)amino, N,N—(C₁₋₆alkyl)₂-amino, N—(C₁₋₆alkyl)-N—(C₁₋₆alkoxy)amino, C₁₋₆alkanoylamino, N—(C₁₋₆alkyl)carbamoyl, N,N—(C₁₋₆alkyl)₂-carbamoyl, C₁₋₆alkylS(O)ₐ wherein a is 0 to 2, C₁₋₆alkoxycarbonyl, C₁₋₆alkoxycarbonylamino, N—(C₁₋₆alkyl)sulphamoyl, N—(C₁₋₆alkyl)-N—(C₁₋₆alkoxy)sulphamoyl, N,N—(C₁₋₆alkyl)₂sulphamoyl, N,N′—(C₁₋₆alkyl)₂ureido, N′,N′—(C₁₋₆alkyl)₂ureido, N—(C₁₋₆alkyl)-N′,N′—(C₁₋₆alkyl)₂ureido, C₁₋₆alkylsulphonylamino, carbocyclyl-R¹⁸— or heterocyclyl-R¹⁹—; wherein R⁸, R^{8a}, and R¹² independently of each other may be optionally substituted on carbon by one or more R²⁰; and wherein if said heterocyclyl contains an —NH— moiety that nitrogen may be optionally substituted by a group selected from R¹;

R¹⁶ in each occurrence is independently selected from halo, nitro, cyano, hydroxy, trifluoromethoxy, amino, carboxy, carbamoyl, mercapto, sulphamoyl, C₁₋₆alkyl, C₂₋₆alkenyl, C₂₋₆alkynyl, C₁₋₆alkoxy, C₁₋₆alkanoyl, C₁₋₆alkanoyloxy, N—(C₁₋₆alkyl)amino, N,N—(C₁₋₆alkyl)₂-amino, C₁₋₆alkanoylamino, N—(C₁₋₆alkyl)carbamoyl, N,N—(C₁₋₆alkyl)₂-carbamoyl, C₁₋₆alkylS(O)ₐ wherein a is 0 to 2, C₁₋₆alkoxycarbonyl, N—(C₁₋₆alkyl)sulphamoyl, N,N—(C₁₋₆alkyl)₂sulphamoyl, C₁₋₆alkylsulphonylamino, carbocyclyl-R²²— or heterocyclyl-R²³—; wherein R¹⁶ may be optionally substituted on carbon by one or more R²⁴; and wherein if said heterocyclyl contains an —NH— moiety that nitrogen may be optionally substituted by a group selected from R²⁵;

R⁶, R⁷, R¹⁰, R¹¹, R¹⁴, R¹⁵, R¹⁸, R¹⁹, R²² and R²³ in each occurrence are independently selected from a direct bond, —O—, —N(R²⁶)—, —C(O)—, —N(R²⁷)C(O)—, —C(O)N(R²⁸)—, —S(O)ₛ—, —SO₂N(R²⁹)— or —N(R³⁰)SO₂—; wherein R²⁶, R²⁷, R²⁸, R²⁹ and R³⁰ are independently selected from hydrogen or C₁₋₆alkyl and s is 0-2;

R⁵, R⁹, R^{9a}, R¹³, R¹⁷, R²¹ and R²⁵ in each occurrence are independently selected from C₁₋₆alkyl, C₁₋₆alkanoyl, C₁₋₆alkylsulphonyl, C₁₋₆alkoxycarbonyl, carbamoyl, N—(C₁₋₆alkyl)carbamoyl, N,N—(C₁₋₆alkyl)carbamoyl, benzyl, benzyloxycarbonyl, benzoyl and phenylsulphonyl;

R²⁰ and R²⁴ in each occurrence are independently selected from halo, nitro, cyano, hydroxy, trifluoromethoxy, trifluoromethyl, amino, carboxy, carbamoyl, mercapto, sulphamoyl, methyl, ethyl, cyclopropyl, cyclobutyl, cyclopentyl, methoxy, ethoxy, acetyl, acetoxy, methylamino, ethylamino, dimethylamino, diethylamino, N-methyl-N-ethylamino, acetylamino, N-methylcarbamoyl, N-ethylcarbamoyl, N,N-dimethylcarbamoyl, N,N-diethylcarbamoyl, N-methyl-N-ethylcarbamoyl, phenyl, methylthio, ethylthio, methylsulphinyl, ethylsulphinyl, mesyl, ethylsulphonyl, methoxycarbonyl, ethoxycarbonyl, N-methylsulphamoyl, N-ethylsulphamoyl, N,N-dimethylsulphamoyl, N,N-diethylsulphamoyl or N-methyl-N-ethylsulphamoyl; wherein R²⁰ and R²⁴ may be optionally substituted on carbon by one or more R⁵⁰; and

R⁵⁰ in each occurrence is independently selected from halo, hydroxy, cyano, and C₁₋₆alkoxy.

Particular values of variable groups contained in formula (I) are as follows. Such values may be used where appropriate with any of the definitions, claims, or embodiments defined hereinabove or hereinbelow.

A is

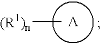

wherein Ring A is selected from aryl, heteroaryl, and carbocyclyl; wherein if said heteroaryl contains an —NH— moiety that nitrogen may be optionally substituted by a group selected from R⁵; or

A is C₁₋₆alkyl; wherein A may be optionally substituted on carbon by one or more R^{8a};

R⁵ is C₁₋₆alkyl;

R^{8a} in each occurrence is independently selected from halo, C₁₋₆alkoxy, and carbocyclyl-R¹⁸—, wherein R^{8a} may be optionally substituted on carbon by one or more R²⁰;

R¹⁸ is a direct bond; and

R²⁰ is methyl.

A is

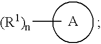

wherein Ring A is selected from phenyl, pyridinyl, cyclopentyl, cyclohexyl, and 1H-pyrazolyl, wherein if said 1H-pyrazolyl contains an —NH— moiety that nitrogen may be optionally substituted by a group selected from R⁵; or

A is selected from ethyl, butyl, 3-methylpentyl, 2-methylbutyl, 3-methylbutyl, sec-butoxymethyl, 2-methylprop-2-yl, but-2-yl, hex-2-yl; wherein said methyl, butyl, 3-methylpentyl, 2-methylbutyl, 3-methylbutyl, sec-butoxymethyl, 2-methylprop-2-yl, but-2-yl, and hex-2-yl may be optionally substituted on carbon by one or more R^{8a};

R⁵ in each occurrence is independently selected from methyl, 2-methylprop-2-yl, and prop-2-yl;

R^{8a} in each occurrence is independently selected from fluoro, 1-methyl-propoxy, cyclopropyl-R¹⁸—, cyclopentyl-R¹⁸—, and cyclohexyl-R¹⁸—; wherein said 1-methyl-propoxy, cyclopropyl-R¹⁸—, cyclopentyl-R¹⁸—, and cyclohexyl-R¹⁸— may be optionally substituted on carbon by one or more R²⁰;

R¹⁸ is a direct bond; and

R²⁰ is methyl.

A is selected from 3-(1-cyano-1-methylethyl)phenyl, 3-(trifluoromethyl)phenyl, 3-chlorophenyl, 3,5-dimethylphenyl, 3-fluoro-5-(trifluoromethyl)phenyl, 3-chloro-5-fluorophenyl, 3-cyclopropyl-5-fluorophenyl, 3,4-dichlorophenyl, 3-cyclopropylphenyl, 3-methylphenyl, 3-methylcyclohexyl, 2,6-dichloropyridin-4-yl, cyclopentyl, 3,4-dimethylphenyl, 6-methylpyridin-2-yl, 3-chloropyridin-4-yl, 5-methylpyridin-3-yl, 1,5-dimethyl-1H-pyrazol-3-yl, 5-methyl-1H-pyrazol-3-yl, 4-methylcyclohexyl, 3-(trifluoromethyl)cyclohexyl, 4,4-difluorocyclohexyl, 1-tert-butyl-5-methyl-1H-pyrazol-3-yl, 1-isopropyl-1H-pyrazol-3-yl, butyl, 3-methylpentyl, 2-methylbutyl, 3-methylbutyl, sec-butoxymethyl, cyclohexylmethyl, 2-methylprop-2-yl, (4-methylcyclohexyl)methyl, but-2-yl, hex-2-yl, cyclopropylmethyl, cyclopentylmethyl, and cyclohexyl(difluoro)methyl.

Ring A is selected from aryl, heteroaryl, and carbocyclyl; wherein if said heteroaryl contains an —NH— moiety that nitrogen may be optionally substituted by a group selected from R⁵; and

R⁵ is C₁₋₆alkyl.

Ring A is selected from phenyl, pyridinyl, cyclopentyl, cyclohexyl, and 1H-pyrazolyl, wherein if said 1H-pyrazolyl contains an —NH— moiety that nitrogen may be optionally substituted by a group selected from R⁵; and

R⁵ in each occurrence is independently selected from methyl, 2-methylprop-2-yl, and prop-2-yl.

Ring A is selected from 3-(1-cyano-1-methylethyl)phenyl, 3-(trifluoromethyl)phenyl, 3-chlorophenyl, 3,5-dimethylphenyl, 3-fluoro-5-(trifluoromethyl)phenyl, 3-chloro-5-fluorophenyl, 3-cyclopropyl-5-fluorophenyl, 3,4-dichlorophenyl, 3-cyclopropylphenyl, 3-methylphenyl, 3-methylcyclohexyl, 2,6-dichloropyridin-4-yl, cyclopentyl, 3,4-dimethylphenyl, 6-methylpyridin-2-yl, 3-chloropyridin-4-yl, 5-methylpyridin-3-yl, 1,5-dimethyl-1H-pyrazol-3-yl, 5-methyl-1H-pyrazol-3-yl, 4-methylcyclohexyl, 3-(trifluoromethyl)cyclohexyl, 4,4-difluorocyclohexyl, 1-tert-butyl-5-methyl-1H-pyrazol-3-yl, and 1-isopropyl-1H-pyrazol-3-yl.

A is selected from C₁₋₆alkyl; wherein said C₁₋₆alkyl may be optionally substituted on carbon by one or more R^{8a};

R^{8a} in each occurrence is independently selected from halo, C₁₋₆alkoxy, and carbocyclyl-R¹⁸—, wherein R^{8a} may be optionally substituted on carbon by one or more R²⁰;

R¹⁸ is a direct bond; and

R²⁰ is methyl.

A is selected from methyl, butyl, 3-methylpentyl, 2-methylbutyl, 3-methylbutyl, sec-butoxymethyl, 2-methylprop-2-yl, but-2-yl, hex-2-yl; wherein said methyl, butyl, 3-methylpentyl, 2-methylbutyl, 3-methylbutyl, sec-butoxymethyl, 2-methylprop-2-yl, but-2-yl, and hex-2-yl may be optionally substituted on carbon by one or more R^{8a};

R^{8a} in each occurrence is independently selected from fluoro, 1-methyl-propoxy, cyclopropyl-R¹⁸—, cyclopentyl-R¹⁸—, and cyclohexyl-R¹⁸—; wherein said 1-methyl-propoxy, cyclopropyl-R¹⁸—, cyclopentyl-R¹⁸—, and cyclohexyl-R¹⁸— may be optionally substituted on carbon by one or more R²⁰;

R¹⁸ is a direct bond; and

R²⁰ is methyl.

A is selected from butyl, 3-methylpentyl, 2-methylbutyl, 3-methylbutyl, sec-butoxymethyl, cyclohexylmethyl, 2-methylprop-2-yl, (4-methylcyclohexyl)methyl, but-2-yl, hex-2-yl, cyclopropylmethyl, cyclopentylmethyl, and cyclohexyl(difluoro)methyl.

X is absent or O.

X is absent.

X is O.

R¹ is a substituent on carbon and is selected from halo, C₁₋₆alkyl, and carbocyclyl-R⁶—; wherein R¹ may be optionally substituted on carbon by one or more R⁸;

R⁶ is a direct bond; and

R⁸ in each occurrence is independently selected from halo and cyano.

R¹ is a substituent on carbon and is selected from fluoro, chloro, methyl, isopropyl, and cyclopropyl-R⁶—; wherein R¹ may optionally be substituted on carbon by one or more R⁸;

R⁶ is a direct bond; and

R⁸ in each occurrence is independently selected from fluoro and cyano.

R¹ is a substituent on carbon and is selected from fluoro, chloro, methyl, trifluoromethyl, 2-cyanoprop-2-yl, and cyclopropyl.

R² is hydrogen.

R² and R³ are independently selected from hydrogen, halo, C₁₋₆alkyl.

R² and R³ are independently selected from hydrogen, chloro, and methyl.

R² is hydrogen and R³ is selected from halo and C₁₋₆alkyl.

R² is hydrogen and R³ is selected from chloro and methyl.

R³ is selected from halo and C₁₋₆alkyl.

R³ is selected from chloro and methyl.

R³ is chloro.

R³ is methyl.

R⁴ is selected from C₁₋₆alkyl, N—(C₁₋₆alkyl)carbamoyl, N—(C₁₋₆alkyl)-N—(C₁₋₆alkoxy)carbamoyl, carbocyclyl-R¹⁴— and heterocyclyl-R¹⁵—; wherein R⁴ may be optionally substituted on carbon by one or more R¹⁶;

R¹⁴ is a direct bond;

R¹⁵ is —C(O)—;

R¹⁶ in each occurrence is independently selected from hydroxy, N—(C₁₋₆alkyl)amino, N,N—(C₁₋₆alkyl)₂-amino, carbocyclyl-R²²— and heterocyclyl-R²³—; wherein R¹⁶ may be optionally substituted on carbon by one or more R²⁴; and wherein if said heterocyclyl contains an —NH— moiety that nitrogen may be optionally substituted by a group selected from R²⁵;

R²² is —N(R⁶)—;

R²³ is a direct bond;

R²⁴ in each occurrence is independently selected from methyl, methoxy, dimethylamino, and cyclopropyl, wherein R²⁴ may be optionally substituted on carbon by one or more R⁵⁰;

R²⁵ is C₁₋₆alkyl;

R²⁶ is hydrogen; and

R⁵⁰ is hydroxy.

R⁴ is selected from methyl, isopropyl, N-methylcarbamoyl, N-methyl-N-methoxycarbamoyl, cyclopropyl-R¹⁴—, and morpholino-R¹⁵—; wherein R⁴ may be optionally substituted on carbon by one or more R¹⁶;

R¹⁴ is a direct bond;

R¹⁵ is —C(O)—;

R¹⁶ in each occurrence is independently selected from hydroxy, methylamino, ethylamino, dimethylamino, N-methyl-N-ethylamino, azetidin-1-yl, morpholino, piperazin-1-yl, piperidin-1-yl, cyclobutyl-R²²—, and cyclopropyl-R²²—; wherein R¹⁶ may be optionally substituted on carbon by one or more R²⁴; and wherein said piperazin-1-yl may be optionally substituted on nitrogen by a group selected from R²⁵;

R²² is —N(R²⁶)—; wherein R²⁶ is hydrogen;

R²⁴ in each occurrence is independently selected from methoxy, dimethylamino, cyclopropyl, cyclobutyl, and cyclopropyl, wherein R²⁴ may be optionally substituted on carbon by one or more R⁵⁰;

R²⁵ is methyl; and

R⁵⁰ is hydroxy.

R⁴ is selected from methyl, isopropyl, N-methylcarbamoyl, (4-methylpiperazin-1-yl)methyl morpholincarbonyl, N-methyl-N-methoxycarbamoyl, hydroxymethyl, (dimethylamino)methyl, 1-hydroxyethyl, piperidinomethyl, (methylamino)methyl, morpholin-4-ylmethyl, 2-(dimethylamino)ethyl, 1-azetidinylmethyl, (cyclobutylainno)methyl, [(cyclopropylmethyl)amino]methyl, [(2-methoxyethyl)methylamino]methyl, [4-(hydroxymethyl)piperidin-1-yl]methyl, isopropyl, (cyclopropylamino)methyl, and cyclopropyl.

m is selected from 0 to 2, wherein the values of R⁴ may be the same or different.

m is selected from 0 and 1.

m is 1.

m is 0.

n is selected from 0 to 2, wherein the values of R¹ may be the same or different.

n is 2, wherein the values of R¹ may be the same or different.

n is selected from 1 and 2, wherein the values of R¹ may be the same or different.

n is 1.

n is 0.

In a further aspect of the invention here is provided a compound of formula (I) (as depicted hereinabove) wherein:

A is

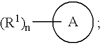

wherein Ring A is selected from aryl, heteroaryl, and carbocyclyl; wherein if said heteroaryl contains an —NH— moiety that nitrogen may be optionally substituted by a group selected from R⁵; or

A is C₁₋₆alkyl; wherein A may be optionally substituted on carbon by one or more R^{8a};

X is absent or O;

R¹ is a substituent on carbon and is selected from halo, C₁₋₆alkyl, and carbocyclyl-R⁶—;

wherein R¹ may be optionally substituted on carbon by one or more R⁸;

R² and R³ are independently selected from hydrogen, halo, C₁₋₆alkyl;

R⁴ is selected from C₁₋₆alkyl, N—(C₁₋₆alkyl)carbamoyl, N—(C₁₋₆alkyl)-N—(C₁₋₆alkoxy)carbamoyl, carbocyclyl-R¹⁴, and heterocyclyl-R¹⁵—; wherein R⁴ may be optionally substituted on carbon by one or more R¹⁶;

R⁵ is C₁₋₆alkyl;

R⁶ is a direct bond;

R⁸ in each occurrence is independently selected from halo and cyano;

R^{8a} in each occurrence is independently selected from halo, C₁₋₆alkoxy, and carbocyclyl-R¹⁸—, wherein R^{8a} may be optionally substituted on carbon by one or more R²⁰;

R¹⁴ is a direct bond;

R¹⁵ is —C(O)—;

R¹⁶ in each occurrence is independently selected from hydroxy, N—(C₁₋₆alkyl)amino, N,N—(C₁₋₆alkyl)₂-amino, carbocyclyl-R²²— and heterocyclyl-R²³—; wherein R¹⁶ may be optionally substituted on carbon by one or more R²⁴; and wherein if said heterocyclyl contains an —NH— moiety that nitrogen may be optionally substituted by a group selected from R²⁵;

R¹⁸ is a direct bond;

R²⁰ is methyl;

R²² is —N(R⁶)—;

R²³ is a direct bond;

R²⁴ in each occurrence is independently selected from methyl, methoxy, dimethylamino, and cyclopropyl, wherein R²⁴ may be optionally substituted on carbon by one or more R⁵⁰;

R²⁵ is C₁₋₆alkyl;

R²⁶ is hydrogen;

R⁵⁰ is hydroxy;

m is selected from 0 to 2, wherein the values of R⁴ may be the same or different; and

n is selected from 0 to 2, wherein the values of R¹ may be the same or different.

A is selected from A is

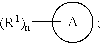

wherein Ring A is selected from phenyl, pyridinyl, cyclopentyl, cyclohexyl, and 1H-pyrazolyl, wherein if said 1H-pyrazolyl contains an —NH— moiety that nitrogen may be optionally substituted by a group selected from R⁵; or

A is selected from ethyl, butyl, 3-methylpentyl, 2-methylbutyl, 3-methylbutyl, sec-butoxymethyl 2-methylprop-2-yl, but-2-yl, and hex-2-yl; wherein said methyl, butyl, 3-methylpentyl, 2-methylbutyl, 3-methylbutyl, sec-butoxymethyl, 2-methylprop-2-yl, but-2-yl, and hex-2-yl may be optionally substituted on carbon by one or more R^{8a};

X is absent or O;

R¹ is a substituent on carbon and is selected from fluoro, chloro, methyl, isopropyl, and cyclopropyl-R⁶—; wherein R¹ may optionally be substituted on carbon by one or more R⁸;

R² and R³ are independently selected from hydrogen, chloro, and methyl;

R⁴ is selected from methyl, isopropyl, N-methylcarbamoyl, N-methyl-N-methoxycarbamoyl, cyclopropyl-R¹⁴—, and morpholino-R¹⁵—; wherein R⁴ may be optionally substituted on carbon by one or more R⁶;

R⁶ is a direct bond;

R⁵ in each occurrence is independently selected from methyl, 2-methylprop-2-yl, and prop-2-yl;

R⁸ in each occurrence is independently selected from fluoro and cyano;

R^{8a} in each occurrence is independently selected from fluoro, 1-methyl-propoxy, cyclopropyl-R¹⁸—, cyclopentyl-R¹⁸— and cyclohexyl-R¹⁸—; wherein said 1-methyl-propoxy, cyclopropyl-R⁸—, cyclopentyl-R¹⁸—, and cyclohexyl-R¹⁸— may be optionally substituted on carbon by one or more R²⁰;

R¹⁴ is a direct bond;

R¹⁵ is —C(O)—;

R¹⁶ in each occurrence is independently selected from hydroxy, methylamino, ethylamino, dimethylamino, N-methyl-N-ethylamino, azetidin-1-yl, morpholino, piperazin-1-yl, piperidin-1-yl, cyclobutyl-R²²—, and cyclopropyl-R²²—; wherein R¹⁶ may be optionally substituted on carbon by one or more R²⁴; and wherein said piperazin-1-yl may be optionally substituted on nitrogen by a group selected from R²⁵;

R¹⁸ is a direct bond;

R²⁰ is methyl;

R²² is —N(R²⁶)—; wherein R²⁶ is hydrogen;

R²⁴ in each occurrence is independently selected from methoxy, dimethylamino, cyclopropyl, cyclobutyl, and cyclopropyl, wherein R²⁴ may be optionally substituted on carbon by one or more R⁵⁰;

R²⁵ is methyl;

R⁵⁰ is hydroxy;

m is selected from 0 and 1; and

n is selected from 0 to 2, wherein the values of R¹ may be the same or different.

A is selected from 3-(1-cyano-1-methylethyl)phenyl, 3-(trifluoromethyl)phenyl, 3-chlorophenyl, 3,5-dimethylphenyl, 3-fluoro-5-(trifluoromethyl)phenyl, 3-chloro-5-fluorophenyl, 3-cyclopropyl-5-fluorophenyl, 3,4-dichlorophenyl, 3-cyclopropylphenyl, 3-methylphenyl, 3-methylcyclohexyl, 2,6-dichloropyridin-4-yl, cyclopentyl, 3,4-dimethylphenyl, 6-methylpyridin-2-yl, 3-chloropyridin-4-yl, 5-methylpyridin-3-yl, 1,5-dimethyl-1H-pyrazol-3-yl, 5-methyl-1H-pyrazol-3-yl, 4-methylcyclohexyl, 3-(trifluoromethyl)cyclohexyl, 4,4-difluorocyclohexyl 1-tert-butyl-5-methyl-1H-pyrazol-3-yl, 1-isopropyl-1H-pyrazol-3-yl, butyl, 3-methylpentyl, 2-methylbutyl, 3-methylbutyl, sec-butoxymethyl, cyclohexylmethyl, 2-methylprop-2-yl, (4-methylcyclohexyl)methyl, but-2-yl, hex-2-yl, cyclopropylmethyl, cyclopentylmethyl, and cyclohexyl(difluoro)methyl;

X is absent or O;

R¹ is a substituent on carbon and is selected from fluoro, chloro, methyl, trifluoromethyl, 2-cyanoprop-2-yl, and cyclopropyl;

R² is hydrogen;

R³ is selected from chloro and methyl;

R⁴ is selected from methyl, isopropyl, N-methylcarbamoyl, (4-methylpiperazin-1-yl)methyl, morpholincarbonyl, N-methyl-N-methoxycarbamoyl, hydroxymethyl, (dimethylamino)methyl, 1-hydroxyethyl, piperidinomethyl, (methylamino)methy, morpholin-4-ylmethyl, 2-(dimethylamino)ethyl, 1-azetidinylmethyl, (cyclobutylamino)methyl, [(cyclopropylmethyl)amino]methyl, [(2-methoxyethyl)methylamino]methyl, [4-(hydroxymethyl)piperidin-1-yl]methyl, isopropyl, (cyclopropylamino)methyl, and cyclopropyl;

m is selected from 0 and 1; and

n is selected from 0 to 2, wherein the values of R¹ may be the same or different.

What is also provided is a compound of formula (Ia):

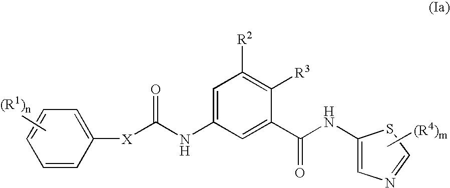

or a pharmaceutically acceptable salt thereof, wherein: R, n, X, R², R³, R⁴, and m are as defined for a compound of formula (I).

What is also provided is a compound of formula (Ib):

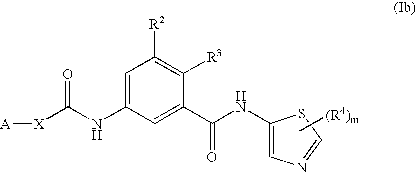

or a pharmaceutically acceptable salt thereof; wherein:

A is

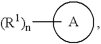

wherein Ring A is heteroaryl; and

R¹, n, X, R², R³, R⁴, and m are as defined for a compound of formula (I).

What is also provided is a compound of formula (Ic):

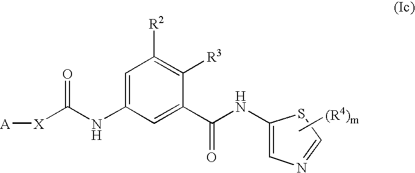

or a pharmaceutically acceptable salt thereof; wherein:

A is

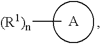

wherein Ring A is carbocyclyl; and

R¹, n, X, R², R³, R⁴, and m are as defined for a compound of formula (I).

What is also provided is a compound of formula (Id):

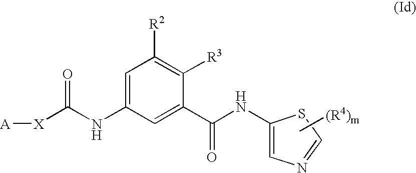

or a pharmaceutically acceptable salt thereof; wherein:

A is

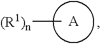

wherein Ring A is heterocyclyl; wherein if said heterocyclyl contains an —NH— moiety that nitrogen may be optionally substituted by a group selected from R⁵; and

R¹, n, X, R², R³, R⁴, R⁵, and m are as defined for a compound of formula (I).

What is also provided is a compound of formula (Ie):

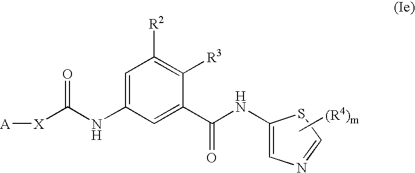

or a pharmaceutically acceptable salt thereof; wherein:

A is C₁₋₆alkyl, C₂₋₆alkenyl, or C₂₋₆alkynyl, each of which may be optionally substituted with 1, 2, or 3 substituents selected from halo, nitro, cyano, hydroxy, trifluoromethoxy, amino, carboxy, carbamoyl, mercapto, sulphamoyl, C₁₋₆alkoxy, C₁₋₆alkanoyl, C₁₋₆alkanoyloxy, N—(C₁₋₆allyl)amino, N,N—(C₁₋₆allyl)₂-amino, C₁₋₆alkalnoylamino, N—(C₁₋₆alkyl)carbamoyl, N,N—(C₁₋₆alkyl)₂-carbamoyl, C₁₋₆alkylS(O)ₐ wherein a is 0 to 2, C₁₋₆alkoxycarbonyl, C₁₋₆alkoxycarbonylamino, N—(C₁₋₆alkyl)sulphamoyl, N,N—(C₁₋₆alkyl)₂sulphamoyl, N—(C₁₋₆alkyl)-N—(C₁₋₆alkoxy)sulphamoyl, N,N′—(C₁₋₆allyl)₂ureido, N′,N′—(C₁₋₆alkyl)₂ureido, N—(C₁₋₆alkyl)-N′,N′—(C₁₋₆alkyl)₂ureido, C₁₋₆alkylsulphonylamino, carbocyclyl-R⁶— or heterocyclyl-R⁷—; wherein A may be optionally substituted on carbon by one or more R⁸; and wherein if said heterocyclyl contains an —NH— moiety that nitrogen may be optionally substituted by a group selected from R⁹; and

X, R², R³, R⁴, R⁶⁻⁸, and m are as defined for a compound of formula (I).

What is also provided is a compound which is:
5-{[3-(1-Cyano-1-methylethyl)benzoyl]amino}-2-methyl-N-(2-methyl-1,3-thiazol-5-yl)benzamide;
2-Chloro-N-1,3-thiazol-5-yl-5-{[3-(trifluoromethyl)benzoyl]amino}benzamide;
2-Chloro-5-[(3-chlorobenzoyl)amino]-N-1,3-thiazol-5-ylbenzamide;
2-Chloro-5-[(3,5-dimethylbenzoyl)amino]-N-1,3-thiazol-5-ylbenzamide;
5-{[3-(1-Cyano-1-methylethyl)benzoyl]amino}-2-methyl-N-1,3-thiazol-5-ylbenzamide;
2-Methyl-N-(2-methyl-1,3-thiazol-5-yl)-5-{[3-(trifluoromethyl)benzoyl]amino}benzamide;
2-Chloro-5-[(3-chlorobenzoyl)amino]-N-(2-methyl-1,3-thiazol-5-yl)benzamide;
2-Chloro-5-[(3,5-dimethylbenzoyl)amino]-N-(2-methyl-1,3-thiazol-5-yl)benzamide;
2-Chloro-N-(2-methyl-1,3-thiazol-5-yl)-5-{[3-(trifluoromethyl)benzoyl]amino}benzamide;
2-Chloro-5-{[3-fluoro-5-(trifluoromethyl)benzoyl]amino}-N-(2-methyl-1,3-thiazol-5-yl)benzamide;
5-[(5-{[3-(1-Cyano-1-methylethyl)benzoyl]amino}-2-methylbenzoyl)amino]-N-methyl-1,3-thiazole-2-carboxamide;
5-{[3-fluoro-5-(trifluoromethyl)benzoyl]amino}-2-methyl-N-(2-methyl-1,3-thiazol-5-yl)benzamide;
5-[(3-Chloro-5-fluorobenzoyl)amino]-2-methyl-N-(2-methyl-1,3-thiazol-5-yl)benzamide;
5-[(3-Cyclopropyl-5-fluorobenzoyl)amino]-2-methyl-N-(2-methyl-1,3-thiazol-5-yl)benzamide;
5-[(3-Chlorobenzoyl)amino]-2-methyl-N-(2-methyl-1,3-thiazol-5-yl)benzamide;
5-[3,4-Dichlorobenzoyl)amino]-2-methyl-N-(2-methyl-1,3-thiazol-5-yl)benzamide;
5-[(3-Cyclopropylbenzoyl)amino]-2-methyl-N-(2-methyl-1,3-thiazol-5-yl)benzamide;
5-[(3,5-Dimethylbenzoyl)amino]-2-methyl-N-(2-methyl-1,3-thiazol-5-yl)benzamide;
2-methyl-5-[(3-methylbenzoyl)amino]-N-(2-methyl-1,3-thiazol-5-yl)benzamide;
2,6-Dichloro-N-(4-methyl-3-{[(2-methyl-1,3-thiazol-5-yl)amino]carbonyl}phenyl)isonicotinamide;
2-Methyl-5-{[(3-methylcyclohexyl)carbonyl]amino}-N-(2-methyl-1,3-thiazol-5-yl)benzamide;
2-Methyl-N-(2-methyl-1,3-thiazol-5-yl)-5-(pentanoylamino)benzamide; or
2-methyl-5-[(4-methylhexanoyl)amino]-N-(2-methyl-1,3-thiazol-5-yl)benzamide.

What is also provided is a pharmaceutical composition which comprises a compound of formula (I), or a pharmaceutically acceptable salt thereof, in association with a pharmaceutically-acceptable diluent or carrier.

What is also provided is a compound of formula (I), or a pharmaceutically acceptable salt thereof, for use as a medicament.

What is also provided is the use of a compound of the formula (I), or a pharmaceutically acceptable salt thereof, in the manufacture of a medicament for use in the production of a CSF-1R kinase inhibitory effect in a warm-blooded animal such as man.

What is also provided is the use of a compound of the formula (I), or a pharmaceutically acceptable salt thereof, in the manufacture of a medicament for use in the production of an anti-cancer effect in a warm-blooded animal such as man.

What is also provided is the use of a compound of the formula (I), or a pharmaceutically acceptable salt thereof, in the manufacture of a medicament for use in the treatment of melanoma, papillary thyroid tumours, cholangiocarcinomas, colon cancer, ovarian cancer, lung cancer, leukaemias, lymphoid malignancies, carcinomas and sarcomas in the liver, kidney, bladder, prostate, breast and pancreas, and primary and recurrent solid tumours of the skin, colon, thyroid, lungs and ovaries.

What is also provided is the use of a compound of formula (I), or a pharmaceutically acceptable salt thereof, in the manufacture of a medicament for use in the treatment of breast, ovarian, bladder, cervical, endometrial, prostate, lung, kidney and pancreatic tumors; haematological malignancies including myelodysplastic syndrome, acute myelogenous leukemia, chronic myelogenous leukemia, non Hodgkin's lymphoma, Hodgkin's disease, multiple myeloma and chronic lymphocytic leukemia; and glioma, squamous cell carcinoma of the esophagus, malignant uveal melanoma and follicular lymphoma, in a warm-blooded animal such as man.

What is also provided is the use of a compound of formula (I), or a pharmaceutically acceptable salt thereof, in the manufacture of a medicament for use in the treatment of tumor-associated osteolysis, osteoporosis including ovariectomy-induced bone loss, orthopedic implant failure, autoimmune disorders including systemic lupus erythematosus, arthritis including rheumatoid arthritis, osteoarthritis, renal inflammation and glomerulonephritis; inflammatory bowel disease; transplant rejection including renal and bone marrow allografts and skin xenograft, atherosclerosis, obesity, Alzheimer's Disease and Langerhans cell histiocytosis, in a warm-blooded animal such as man.

What is also provided is a method for producing a CSF-1R kinase inhibitory effect in a warm-blooded animal, such as man, in need of such treatment, said method comprising administering to said animal an effective amount of a compound of formula (I), or a pharmaceutically acceptable salt thereof.

What is also provided is a method for producing an anti-cancer effect in a warm-blooded animal, such as man, in need of such treatment, said method comprising administering to said animal an effective amount of a compound of formula (I), or a pharmaceutically acceptable salt thereof.

What is also provided is a method of treating melanoma, papillary thyroid tumours, cholangiocarcinomas, colon cancer, ovarian cancer, lung cancer, leukaemias, lymphoid malignancies, carcinomas and sarcomas in the liver, kidney, bladder, prostate, breast and pancreas, and primary and recurrent solid tumours of the skin, colon, thyroid, lungs and ovaries, in a warm-blooded animal, such as man, in need of such treatment which comprises administering to said animal an effective amount of a compound of formula (I) or a pharmaceutically acceptable salt thereof.

What is also provided is a method for treating breast, ovarian, bladder, cervical, endometrial, prostate, lung, kidney and pancreatic tumors; haematological malignancies including myelodysplastic syndrome, acute myelogenous leukemia, chronic myelogenous leukemia, non Hodgkin's lymphoma, Hodgkin's disease, multiple myeloma and chronic lymphocytic leukemia; and glioma, squamous cell carcinoma of the esophagus, malignant uveal melanoma and follicular lymphoma, in a warm-blooded animal, such as man, in need of such treatment, said method comprising administering to said animal an effective amount of a compound of formula (I), or a pharmaceutically acceptable salt thereof.

What is also provided is a method for treating tumor-associated osteolysis, osteoporosis including ovariectomy-induced bone loss, orthopedic implant failure, autoimmune disorders including systemic lupus erythematosus, arthritis including rheumatoid arthritis, osteoarthritis, renal inflammation and glomerulonephritis; inflammatory bowel disease; transplant rejection including renal and bone marrow allografts and skin xenograft, atherosclerosis, obesity, Alzheimer's Disease and Langerhans cell histiocytosis, in a warm blooded animal, such as man, in need of such treatment, said method comprising administering to said animal an effective amount of a compound of formula (I), or a pharmaceutically salt thereof.

What is also provided is a pharmaceutical composition which comprises a compound of the formula (I), or a pharmaceutically acceptable salt thereof, in association with a pharmaceutically-acceptable diluent or carrier for use in the production of a CSF-1R kinase inhibitory effect in a warm-blooded animal such as man.

What is also provided is a pharmaceutical composition which comprises a compound of the formula (I), or a pharmaceutically acceptable salt thereof, in association with a pharmaceutically-acceptable diluent or carrier for use in the production of an anti-cancer effect in a warm-blooded animal such as man.

What is also provided is a pharmaceutical composition which comprises a compound of the formula (I) or a pharmaceutically acceptable salt thereof, in association with a pharmaceutically-acceptable diluent or carrier for use in the treatment of melanoma, papillary thyroid tumors, cholangiocarcinomas, colon cancer, ovarian cancer, lung cancer, leukaemias, lymphoid malignancies, carcinomas and sarcomas in the liver, kidney, bladder, prostate, breast and pancreas, and primary and recurrent solid tumours of the skin, colon, thyroid, lungs and ovaries in a warm-blooded animal such as man.

What is also provided is a pharmaceutical composition comprising a compound of formula (I), or a pharmaceutically acceptable salt thereof, and at least one pharmaceutically acceptable diluent or carrier, for use in the treatment of breast, ovarian, bladder, cervical, endometrial, prostate, lung, kidney and pancreatic tumors; haematological malignancies including myelodysplastic syndrome, acute myelogenous leukemia, chronic myelogenous leukemia, non Hodgkin's lymphoma, Hodgkin's disease, multiple myeloma and chronic lymphocytic leukemia; and glioma, squamous cell carcinoma of the esophagus, malignant uveal melanoma and follicular lymphoma, in a warm-blooded animal such as man.

What is also provided is a pharmaceutical composition comprising a compound of formula (I), or a pharmaceutically acceptable salt thereof, and at least one pharmaceutically acceptable diluent or carrier, for use in the treatment of tumor-associated osteolysis, osteoporosis including ovariectomy-induced bone loss, orthopedic implant failure, autoimmune disorders including systemic lupus erythematosus, arthritis including rheumatoid arthritis, osteoarthritis, renal inflammation and glomerulonephritis; inflammatory bowel disease; transplant rejection including renal and bone marrow allografts and skin xenograft, atherosclerosis, obesity, Alzheimer's Disease and Langerhans cell histiocytosis, in a warm-blooded animal such as man.

What is also provided is a compound of formula (I), or a pharmaceutically acceptable salt thereof, for use in the production of a CSF-1R kinase inhibitory effect in a warm-blooded animal such as man.

What is also provided is a compound of formula (I), or a pharmaceutically acceptable salt thereof, for use in the production of an anti-cancer effect in a warm-blooded animal such as man.

What is also provided is a compound of formula (I), or a pharmaceutically acceptable salt thereof, for use in the treatment of breast, ovarian, bladder, cervical, endometrial, prostate, lung, kidney and pancreatic tumors; haematological malignancies including myelodysplastic syndrome, acute myelogenous leukemia, chronic myelogenous leukemia, non Hodgkin's lymphoma, Hodgkin's disease, multiple myeloma and chronic lymphocytic leukemia; and glioma, squamous cell carcinoma of the esophagus, malignant uveal melanoma and follicular lymphoma, in a warm-blooded animal such as man.

What is also provided is a compound of formula (I), or a pharmaceutically acceptable salt thereof, for use in the treatment of tumor-associated osteolysis, osteoporosis including ovariectomy-induced bone loss, orthopedic implant failure, autoimmune disorders including systemic lupus erythematosus, arthritis including rheumatoid arthritis, osteoarthritis, renal inflammation and glomerulonephritis; inflammatory bowel disease; transplant rejection including renal and bone marrow allografts and skin xenograft, atherosclerosis, obesity, Alzheimer's Disease and Langerhans cell histiocytosis, in a warm-blooded animal such as man.

What is also provided is a process for preparing a compound of formula (I) or a pharmaceutically acceptable salt thereof, comprising:

Process a-1) Reacting an amine of the formula (A)

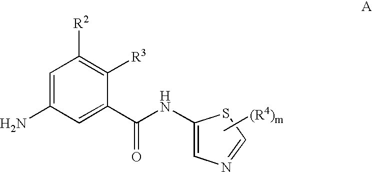

with an acid of formula B or an activated acid derivative thereof:

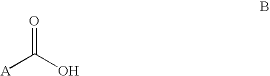

Process a-2) Reacting an amine of the formula (A)

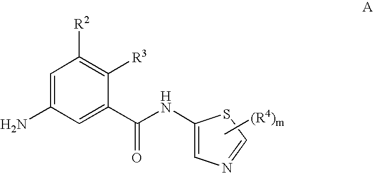

with R—N═C═O, wherein R is C₁₋₆alkyl, aryl, aralkyl, heteroaralkyl, or heteroaryl;
   Process a-3) Reacting an amine of the formula (A)

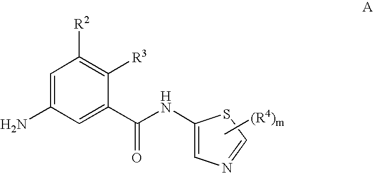

with a chloroformate or an activating agent (e.g., carbonyl diimidazole, phosgene, or another reagent known to the skilled artisan), followed by ROH or RR′NH, wherein R is C₁₋₆alkyl, aryl, heteroaryl, aralkyl, or heteroaralkyl and R¹ is H or C₁₋₆alkyl;
   Process b) Reacting an acid of formula C or an activated acid derivative thereof:

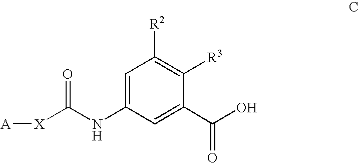

with an amine of formula D:

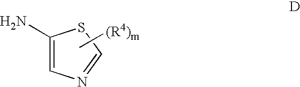

and thereafter if necessary:

i) converting a compound of formula (I) into another compound of formula (I);

ii) removing any protecting groups;

iii) forming a pharmaceutically acceptable salt.

### DETAILED DESCRIPTION OF THE INVENTION

### Definitions

In this specification the term “alkyl” includes both straight and branched chain alkyl groups. References to individual alkyl groups such as “propyl” are specific for the straight chain version only and references to individual branched chain alkyl groups such as ‘isopropyl’ are specific for the branched chain version only. For example, “C₁₋₆alkyl” includes C₁₋₄alkyl, C₁₋₃alkyl, propyl, isopropyl and t-butyl. A similar convention applies to other radicals, for example “phenylC₁₋₆alkyl” includes phenylC₁₋₄alkyl, benzyl, 1-phenylethyl and 2-phenylethyl.

The term “halo” refers to fluoro, chloro, bromo and iodo.

Where optional substituents are chosen from “one or more” groups it is to be understood that this definition includes all substituents being chosen from one of the specified groups or the substituents being chosen from two or more of the specified groups.

“Heterocyclyl” means a saturated or partially saturated monocyclic, fused, bridged, or spiro bicyclic heterocyclic ring system(s). Monocyclic heterocyclic rings contain from about 3 to 12 ring atoms, with from 1 to 5 heteroatoms selected from N, O, and S, and preferably from 3 to 7 member atoms, in the ring. Bicyclic heterocycles contain from 7 to 17 member atoms, preferably 7 to 12 member atoms, in the ring. Bicyclic heterocycles contain from about 7 to about 17 ring atoms, preferably from 7 to 12 ring atoms. Bicyclic heterocyclic(s) rings may be fused, spiro, or bridged ring systems. Examples of heterocyclic groups include cyclic ethers (oxiranes) such as ethyleneoxide, tetrahydrofuran, dioxane, and substituted cyclic ethers, wherein the substituents are as specified. Typical substituted cyclic ethers include propyleneoxide, phenyloxirane (styrene oxide), cis-2-butene-oxide (2,3-dimethyloxirane), 3-Chlorotetrahydrofuran, 2,6-dimethyl-1,4-dioxane, and the like. Heterocycles containing nitrogen are groups such as pyrrolidine, piperidine, piperazine, tetrahydrotriazine, tetrahydropyrazole, and substituted groups such as 3-aminopyrrolidine, 4-methylpiperazin-1-yl, and the like. Typical sulfur containing heterocycles include tetrahydrothiophene, dihydro-1,3-dithiol-2-yl, and hexahydrothiepin-4-yl. Other commonly employed heterocycles include dihydro-oxathiol-4-yl, tetrahydro-oxazolyl, tetrahydro-oxadiazolyl, tetrahydrodioxazolyl, tetrahydro-oxathiazolyl, hexahydrotriazinyl, tetrahydro-oxazinyl, morpholinyl, thiomorpholinyl, tetrahydropyrimidinyl, dioxolinyl, octahydrobenzofuranyl, octahydrobenzimidazolyl, and octahydrobenzothiazolyl. For heterocycles containing sulfur, the oxidized sulfur heterocycles containing SO or SO₂ groups are also included. Examples include the sulfoxide and sulfone forms of tetrahydrothiophene.

“Carbocyclyl” is a saturated or partially saturated, hydrocarbon ring containing from 3 to 6 carbon atoms, for example, cyclopropyl, cyclobutyl, cyclopentyl, cyclohexyl. Where possible, the cycloalkyl group may contain double bonds, for example, 3-cyclohexen-1-yl.

The term “aryl” means a cyclic or polycyclic aromatic ring having from 5 to 12 carbon atoms. The term aryl includes both monovalent species and divalent species. Examples of aryl groups include, but are not limited to, phenyl, biphenyl, naphthyl, 2-chlorophenyl, 3-chlorophenyl, 4-chlorophenyl, 2-methylphenyl, 3-methylphenyl, 4-methylphenyl, 2-methoxyphenyl, 3-methoxyphenyl, 4-methoxyphenyl, 2-chloro-3-methylphenyl, 2-chloro-4-methylphenyl, 2-chloro-5-methylphenyl, 3-chloro-2-methylphenyl, 3-chloro-4-methylphenyl, 4-chloro-2-methylphenyl, 4-chloro-3-methylphenyl, 5-chloro-2-methylphenyl, 2,3-dichlorophenyl, 2,5-dichlorophenyl, 3,4-dichlorophenyl, 2,3-dimethylphenyl, 3,4-dimethylphenyl, 4-trifluoromethyl and the like.

“Alkylene” means a group that is positioned between and serves to connect two other chemical groups. Thus, “(C₁-C₆)alkylene” means a linear saturated divalent hydrocarbon radical of one to six carbon atoms or a branched saturated divalent hydrocarbon radical of three to six carbon atoms, e.g., methylene, ethylene, propylene, 2-methylpropylene, pentylene, and the like.

Aralkyl means an aryl group covalently attached to a (C₁-C₆)alkylene group, both of which are defined herein. Examples of aralykl groups include benzyl, phenylethyl, 3-(3-chlorophenyl)-2-methylpentyl, and the like.

The term “heteroaryl” means an aromatic mono-, bi-, or polycyclic ring incorporating one or more (i.e. 1-4) heteroatoms selected from N, O, and S. The term heteroaryl includes both monovalent species and divalent species. Examples of monocyclic heteroaryl include, but are not limited to substituted or unsubstituted thienyl, furanyl, pyrrolyl, imidazolyl, pyrazolyl, isothiazolyl, isoxazolyl, triazolyl, tetrazolyl, pyridinyl, pyrazinyl, pyrimidinyl, piperidinyl, pyrrolidinyl, piperazinyl, azetidinyl, aziridinyl, morpholinyl, thietanyl, oxetaryl. Monocyclic diheterocycles include, but are not limited to, 5-imidazolyl, pyrazolyl, isothiazolyl, isoxazolyl, triazolyl, tetrazolyl, pyrazinyl, pyrimidinyl, piperazinyl, morpholinyl. Examples of bicyclic and polyclic heteroaryl groups include, but are not limited to include but are not limited to indolizinyl, isoindolyl, indolyl, indazolyl, purinyl, quinolizinyl, quinolinyl, isoquinolinyl, phthalazinyl, naphthyridinyl, quinazolinyl, cinnolinyl, pteridinyl, carbazolyl, carbazolyl, carbolinyl, phenanthridinyl, acridinyl, perimidinyl, phenathrolinyl, phenazinyl, phenothiazinyl, phenoxazinyl, benzisoquinolinyl, thieno[2,3-b]furanyl, pyrazino[2,3-c]carbazolyl, furo[3,2-b]-pyranyl, pyrido[2,3-d]-o-oxazinyl, pyrazolo[4,3-d]-oxazolyl, imidazo[4,5-d]thiazolyl, pyrazino[2,3-d]pyridazinyl, imidazo[2,1-b]thiazolyl, furo[3,4-c]cinnolinyl, 4H-pyrido[2,3-c]carbazolyl, imidazo[1,2-b][1,2,4]triazinyl, 7-benzo[b]thienyl, benzoxazolyl, benzimidazolyl, benzothiazolyl, benzoxapinyl, benzoxazinyl, 1H-pyrrolo[1,2-b][2]benzazapinyl. Typical fused heteroaryl groups include, but are not limited to quinolinyl, isoquinolinyl, indolyl, benzo[b]thienyl, benzoxazolyl, benzimidazolyl, benzothiazolyl.

“Heteroaralkyl” means an heteroaryl group covalently attached to a (C₁-C₆)alkylene group, both of which are defined herein. Examples of heteroaralkyl groups include pyridin-3-ylmethyl, 3-(benzofuran-2-yl)propyl, and the like.

An example of “C₁₋₆alkanoyloxy” is acetoxy.

Examples of “C₁₋₆alkoxycarbonyl” include methoxycarbonyl, ethoxycarbonyl, n- and t-butoxycarbonyl.

Examples of “C₁₋₆alkoxy” include methoxy, ethoxy and propoxy.

Examples of “C₁₋₆alkanoylamino” include formamido, acetamido and propionylamino.

Examples of “C₁₋₆alkylS(O)ₐ wherein a is 0 to 2” include methylthio, ethylthio, methylsulphinyl, ethylsulphinyl, mesyl and ethylsulphonyl.

Examples of “C₁₋₆alkanoyl” include propionyl and acetyl.

Examples of “N—(C₁₋₆alkyl)amino” include methylamino and ethylamino.

Examples of “N,N—(C₁₋₆alkyl)₂-amino” include di-N-methylamino, di-(N-ethyl)amino and N-ethyl-N-methylamino.

Examples of “C₂₋₆alkenyl” are vinyl, allyl and 1-propenyl.

Examples of “C₂₋₆alkynyl” are ethynyl, 1-propynyl and 2-propynyl.

Examples of “N—(C₁₋₆alkyl)sulphamoyl” are N-(methyl)sulphamoyl and N-(ethyl)sulphamoyl.

Examples of “N—(C₁₋₆alkyl)₂sulphamoyl” are N,N-(dimethyl)sulphamoyl and N-(methyl)-N-(ethyl)sulphamoyl.

Examples of “N—(C₁₋₆alkyl)carbamoyl” are N—(C₁₋₄alkyl)carbamoyl, methylaminocarbamoyl and ethylaminocarbamoyl.

Examples of “N,N—(C₁₋₆alkyl)₂-carbamoyl” are N,N—(C₁₋₄alkyl)₂-carbamoyl, dimethylaminocarbamoyl and methylethylaminocarbamoyl.

Examples of “C₁₋₆alkylsulphonyl” are mesyl, ethylsulphonyl and isopropylsulphonyl.

Examples of “C₁₋₆alkylsulphonylamino” are mesylamino, ethylsulphonylamino and isopropylsulphonylamino.

Examples of “C₁₋₆alkoxycarbonylamino” are methoxycarbonylamino and t-butoxycarbonylamino.

Examples of “N—(C₁₋₆alkyl)-N—(C₁₋₆alkoxy)sulphamoyl” are N-(methyl)-N-(methoxy)sulphamoyl and N-(ethyl)-N-(propoxy)sulphamoyl.

Examples of “N,N′—(C₁₋₆alkyl)₂ureido” are N,N′-dimethylureido and N-methyl-N′-propylureido.

Examples of “N′,N′—(C₁₋₆alkyl)₂ureido” are N′,N′-diethylureido and N′-methyl-N′-propylureido.

Example of “N—(C₁₋₆allyl)-N′,N′—(C₁₋₆alkyl)₂ureido” are N-(methyl)-N′-ethyl-N′-isopropylureido and N-ethyl-N′,N′-diethylureido.

Examples of “N—(C₁₋₆alkyl)-N—(C₁₋₆alkoxy)amino” are N-(methyl)-N-(propoxy)amino and N-methyl-N-methoxyamino.

A suitable pharmaceutically acceptable salt of a compound of the invention is, for example, an acid-addition salt of a compound of the invention which is sufficiently basic, for example, an acid-addition salt with, for example, an inorganic or organic acid, for example hydrochloric, hydrobromic, sulphuric, phosphoric, trifluoroacetic, citric or maleic acid. In addition a suitable pharmaceutically acceptable salt of a compound of the invention which is sufficiently acidic is an alkali metal salt, for example a sodium or potassium salt, an alkaline earth metal salt, for example a calcium or magnesium salt, an ammonium salt or a salt with an organic base which affords a physiologically-acceptable cation, for example a salt with methylamine, dimethylamine, trimethylamine, piperidine, morpholine or tris-(2-hydroxyethyl)amine.

Some compounds of the formula (I) may have chiral centres and/or geometric isomeric centres (E- and Z-isomers), and it is to be understood that the invention encompasses all such optical, diastereoisomers and geometric isomers that possess CSF-1R kinase inhibitory activity. The invention further relates to any and all tautomeric forms of the compounds of the formula (I) that possess CSF-1R kinase inhibitory activity.

It is also to be understood that certain compounds of the formula (I) can exist in solvated as well as unsolvated forms such as, for example, hydrated forms. It is to be understood that the invention encompasses all such solvated forms that possess CSF-1R kinase inhibitory activity.

Preparation of Invention Compounds

Another aspect of the present invention provides a process for preparing a compound of formula (I) or a pharmaceutically acceptable salt thereof, comprising:

Process a-1) Reacting an amine of the formula (A)

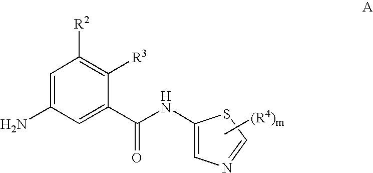

with an acid of formula B or an activated acid derivative thereof:

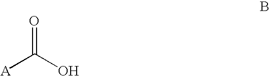

Process a-2) Reacting an amine of the formula (A)

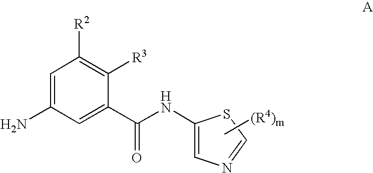

with R—N═C═O, wherein R is C₁₋₆alkyl, aryl, aralkyl, heteroaralkyl, or heteroaryl;
   Process a-3) Reacting an amine of the formula (A)

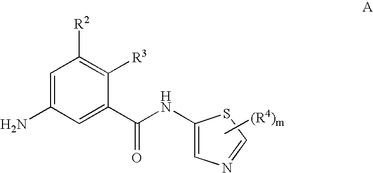

with a chloroformate or an activating agent (e.g., carbonyl diimidazole, phosgene, or another reagent known to the skilled artisan), followed by ROH or RR′NH, wherein R is C₁₋₆alkyl, aryl, heteroaryl, aralkyl, or heteroaralkyl and R′ is H or C₁₋₆alkyl;
   Process b) Reacting an acid of formula C or an activated acid derivative thereof:

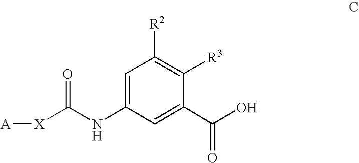

with an amine of formula D:

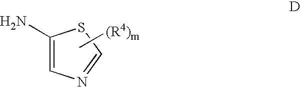

and thereafter if necessary:

i) converting a compound of formula (I) into another compound of formula (I);

ii) removing any protecting groups;

iii) forming a pharmaceutically acceptable salt.

Specific reaction conditions for the above reactions are as follows.

### Process a) and Process b)

Amines and acids may be coupled together in the presence of a suitable coupling reagent. Standard peptide coupling reagents known in the art can be employed as suitable coupling reagents, or for Example carbonyldiimidazole and dicyclohexyl-carbodiimide, optionally in the presence of a catalyst such as dimethylaminopyridine or 4-pyrrolidinopyridine, optionally in the presence of a base for Example triethylamine, pyridine, or 2,6-di-alkyl-pyridines such as 2,6-lutidine or 2,6-di-tert-butylpyridine. Suitable solvents include dimethylacetamide, dichloromethane, benzene, tetrahydrofuran and dimethylformamide. The coupling reaction may conveniently be performed at a temperature in the range of −40 to 40° C.

Suitable activated acid derivatives include acid halides, for Example acid chlorides, and active esters, for Example pentafluorophenyl esters. The reaction of these types of compounds with amines is well known in the art, for Example they may be reacted in the presence of a base, such as those described above, and in a suitable solvent, such as those described above. The reaction may conveniently be performed at a temperature in the range of −40 to 40° C.

Amines of formula A may be prepared according to Scheme 1.

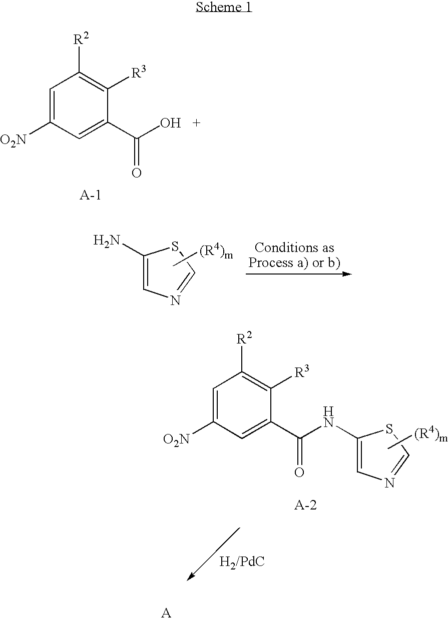

An alternative to the Scheme 1 approach commencing from the corresponding amino compound is depicted in Scheme 2.

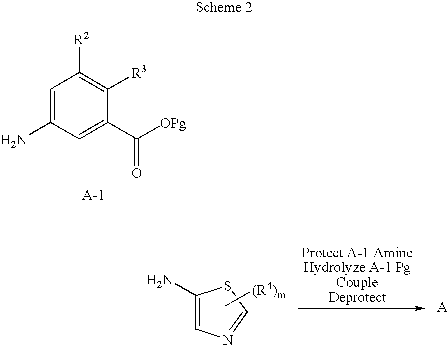

Acids of formula C may be prepared according to Scheme 3.

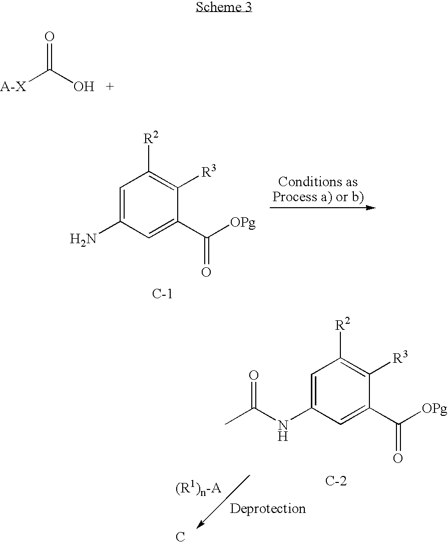

Wherein Pg is an acid protecting group, for example such as those described herein below.

It will be appreciated that certain of the various ring substituents in the compounds of the present invention may be introduced by standard aromatic substitution reactions or generated by conventional functional group modifications either prior to or immediately following the processes mentioned above, and as such are included in the process aspect of the invention. Such reactions and modifications include, for example, introduction of a substituent by means of an aromatic substitution reaction, reduction of substituents, alkylation of substituents and oxidation of substituents. The reagents and reaction conditions for such procedures are well known in the chemical art. Particular examples of aromatic substitution reactions include the introduction of a nitro group using concentrated nitric acid, the introduction of an acyl group using, for example, an acyl halide and Lewis acid (such as aluminium trichloride) under Friedel Crafts conditions; the introduction of an alkyl group using an alkyl halide and Lewis acid (such as aluminium trichloride) under Friedel Crafts conditions; and the introduction of a halogeno group. Particular examples of modifications include the reduction of a nitro group to an amino group by for example, catalytic hydrogenation with a nickel catalyst or treatment with iron in the presence of hydrochloric acid with heating; oxidation of alkylthio to alkylsulphinyl or alkylsulphonyl.

It will also be appreciated that in some of the reactions mentioned herein it may be necessary/desirable to protect any sensitive groups in the compounds. The instances where protection is necessary or desirable and suitable methods for protection are known to those skilled in the art. Conventional protecting groups may be used in accordance with standard practice (for illustration see T. W. Green, Protective Groups in Organic Synthesis, John Wiley and Sons, 1991). Thus, if reactants include groups such as amino, carboxy or hydroxy it may be desirable to protect the group in some of the reactions mentioned herein.

A suitable protecting group for an amino or alkylamino group is, for example, an acyl group, for example an alkanoyl group such as acetyl, an alkoxycarbonyl group, for example a methoxycarbonyl, ethoxycarbonyl or t-butoxycarbonyl group, an arylmethoxycarbonyl group, for example benzyloxycarbonyl, or an aroyl group, for example benzoyl. The deprotection conditions for the above protecting groups necessarily vary with the choice of protecting group. Thus, for example, an acyl group such as an alkanoyl or alkoxycarbonyl group or an aroyl group may be removed for example, by hydrolysis with a suitable base such as an alkali metal hydroxide, for example lithium or sodium hydroxide. Alternatively an acyl group such as a t-butoxycarbonyl group may be removed, for example, by treatment with a suitable acid as hydrochloric, sulphuric or phosphoric acid or trifluoroacetic acid and an arylmethoxycarbonyl group such as a benzyloxycarbonyl group may be removed, for example, by hydrogenation over a catalyst such as palladium-on-carbon, or by treatment with a Lewis acid for example boron tris(trifluoroacetate). A suitable alternative protecting group for a primary amino group is, for example, a phthaloyl group which may be removed by treatment with an alkylamine, for example dimethylaminopropylamine, or with hydrazine.

A suitable protecting group for a hydroxy group is, for example, an acyl group, for example an alkanoyl group such as acetyl, an aroyl group, for example benzoyl, or an arylmethyl group, for example benzyl. The deprotection conditions for the above protecting groups will necessarily vary with the choice of protecting group. Thus, for example, an acyl group such as an alkanoyl or an aroyl group may be removed, for example, by hydrolysis with a suitable base such as an alkali metal hydroxide, for example lithium or sodium hydroxide. Alternatively an arylmethyl group such as a benzyl group may be removed, for example, by hydrogenation over a catalyst such as palladium-on-carbon.

A suitable protecting group for a carboxy group is, for example, an esterifying group, for example a methyl or an ethyl group which may be removed, for example, by hydrolysis with a base such as sodium hydroxide, or for example a t-butyl group which may be removed, for example, by treatment with an acid, for example an organic acid such as trifluoroacetic acid, or for example a benzyl group which may be removed, for example, by hydrogenation over a catalyst such as palladium-on-carbon.

The protecting groups may be removed at any convenient stage in the synthesis using conventional techniques well known in the chemical art.

As stated hereinbefore the compounds defined in the present invention possess anti-cancer activity which is believed to arise from the CSF-1R kinase inhibitory activity of the compounds. These properties may be assessed, for example, using the procedure set out below.

### Biological Activity

CSF-1R In Vitro AlphaScreen Assay

Activity of purified CSF-1R was determined in vitro using an Amplified Luminescent Proximity Homogeneous Assay (ALPHA)(Perkin Elmer), which measures phosphorylation of the CSF-1R substrate, biotinylated poly-glutamine-tyrosine peptide (pEY-HTRF CisBio 61GT0BLD), as described below. The His-tagged kinase domain of CSF-1R (i.e., amino acids 568-912, GeneBank ID NM_{—}005211; (see page 25 lines 13-19 of WO 2006/067445 for the sequence listing)) was purified from baculovirus infected SF+Express insect cells (1.4×106 cells/ml), French pressed and chromatographed through subsequent Qiagen Ni-NTA, Superflow Mono Q HR 10/10, and Superdex 200 SEC columns. Typical yield was 322 ug/l of cell pellet at >95% purity.

The phosphorylation of the CSF-1R substrate in the presence and absence of the compound of interest was determined. Briefly, 0.2 pM of purified CSF-1R, 5 nM pEY substrate, and compound were preincubated in 1× buffer for 30 minutes at 25° C. Reactions were initiated with addition of 90 μM adenosine triphosphate (ATP) in 1× buffer and incubated at 25° C. for 40 minutes and reactions stopped by addition of 5 μl of detection mix consisting of 136 mM NaCl, 102 mM ethylenediamine tetraacetic acid, 1.65 mg/ml BSA, 40 ug/ml Streptavidin donor beads (Perkin Elmer 6760620M), 40 ug/ml pEY100 acceptor beads (Perkin Elmer 6760620M). Plates were incubated at 25° C. for 18 hours in the dark. Phosphorylated substrate was detected by an EnVision plate reader (Perkin Elmer) 680 nm excitation, 520-620 nm emission. Data was graphed and IC₅₀s calculated using Excel Fit (Microsoft).

When tested in the above in vitro assay, the compounds of the present invention exhibited activity less than 30 μM. For example the following results were obtained:

| Example No. | IC₅₀ (nM) |
|---|---|
| 15 | 7 nM |
| 20 | 10 nM |
| 21 | 13 nM |

### Pharmaceutical Formulations

According to a further aspect of the invention there is provided a pharmaceutical composition which comprises a compound of the formula (I), or a pharmaceutically acceptable salt thereof, as defined herein, in association with a pharmaceutically-acceptable diluent or carrier.

The composition may be in a form suitable for oral administration, for example as a tablet or capsule, for parenteral injection (including intravenous, subcutaneous, intramuscular, intravascular or infusion) as a sterile solution, suspension or emulsion, for topical administration as an ointment or cream or for rectal administration as a suppository.

In general the above compositions may be prepared in a conventional manner using conventional excipients.

The compound of formula (I) will normally be administered to a warm-blooded animal at a unit dose within the range 1-1000 mg/kg, and this normally provides a therapeutically-effective dose. Preferably a daily dose in the range of 10-100 mg/kg is employed. However the daily dose will necessarily be varied depending upon the host treated, the particular route of administration, and the severity of the illness being treated. Accordingly the optimum dosage may be determined by the practitioner who is treating any particular patient.

### Uses

According to a further aspect of the present invention there is provided a compound of the formula (I), or a pharmaceutically acceptable salt thereof, as defined hereinbefore for use in a method of treatment of the human or animal body by therapy.

We have found that the compounds defined in the present invention, or a pharmaceutically acceptable salt thereof, are effective anti-cancer agents which property is believed to arise from their CSF-1R kinase inhibitory properties. Accordingly the compounds of the present invention are expected to be useful in the treatment of diseases or medical conditions mediated alone or in part by CSF-1R kinase, i.e. the compounds may be used to produce a CSF-1R kinase inhibitory effect in a warm-blooded animal in need of such treatment.

Thus the compounds of the present invention provide a method for treating cancer characterised by inhibition of CSF-1R kinase, i.e. the compounds may be used to produce an anti-cancer effect mediated alone or in part by the inhibition of CSF-1R kinase.

Such a compound of the invention is expected to possess a wide range of anti-cancer properties as aberrant expression of CSF1R and/or CSF1 has been observed in multiple human cancers and derived cell lines, including but not limited to, breast, ovarian, endometrial, prostate, lung, kidney and pancreatic tumors as well as haematological malignancies including, but not limited to, myelodysplastic syndrome, acute myelogenous leukemia, chronic myelogenous leukemia, non Hodgkin's lymphoma, Hodgkin's disease, multiple myeloma and chronic lymphocytic leukemia. Activating mutations have also been reported in haematopoietic and lymphoid tissue and lung cancer. Further, tumor associated macrophages have been associated with poor prognosis in multiple tumor types including, but not limited to, breast, endometrial, kidney, lung, bladder and cervical cancers, glioma, squamous cell carcinoma of the esophagus, malignant uveal melanoma and follicular lymphoma. It is expected that a compound of the invention will possess anticancer activity against these cancers through direct effect on the tumor and/or indirectly through effect on tumor associated macrophages. Alternatively particular cancers include melanoma, papillary thyroid tumours, cholangiocarcinomas, colon cancer, ovarian cancer, lung cancer, leukaemias, lymphoid malignancies, carcinomas and sarcomas in the liver, kidney, bladder, prostate, breast and pancreas, and primary and recurrent solid tumours of the skin, colon, thyroid, lungs and ovaries.

In a further aspect of the invention, compounds of formula (I) may be also be of value in the treatment of certain additional indications. These indications include, but are not limited to tumor-associated osteolysis, osteoporosis including ovariectomy-induced bone loss, orthopedic implant failure, autoimmune disorders including systemic lupus erythematosus, arthritis including rheumatoid arthritis, osteoarthritis, renal inflammation and glomerulonephritis; inflammatory bowel disease; transplant rejection including renal and bone marrow allografts and skin xenograft, atherosclerosis, obesity, Alzheimer's Disease and Langerhans cell histiocytosis. A further aspect of the present invention therefore includes the treatment of one of more of these diseases, particularly arthritis including rheumatoid arthritis and osteoarthritis.

Thus according to this aspect of the invention there is provided a compound of the formula (I), or a pharmaceutically acceptable salt thereof, as defined hereinbefore for use as a medicament.

According to a further aspect of the invention there is provided the use of a compound of the formula (I), or a pharmaceutically acceptable salt thereof, as defined hereinbefore in the manufacture of a medicament for use in the production of a CSF-1R kinase inhibitory effect in a warm-blooded animal such as man.

According to this aspect of the invention there is provided the use of a compound of the formula (I), or a pharmaceutically acceptable salt thereof, as defined hereinbefore, in the manufacture of a medicament for use in the production of an anti-cancer effect in a warm-blooded animal such as man.

According to a further feature of the invention, there is provided the use of a compound of the formula (I), or a pharmaceutically acceptable salt thereof, as defined herein before in the manufacture of a medicament for use in the treatment of melanoma, papillary thyroid tumours, cholangiocarcinomas, colon cancer, ovarian cancer, lung cancer, leukaemias, lymphoid malignancies, carcinomas and sarcomas in the liver, kidney, bladder, prostate, breast and pancreas, and primary and recurrent solid tumours of the skin, colon, thyroid, lungs and ovaries.

According to a further feature of the invention, there is provided the use of a compound of formula (I), or a pharmaceutically acceptable salt thereof, as defined herein before in the manufacture of a medicament for use in the treatment of breast, ovarian, bladder, cervical, endometrial, prostate, lung, kidney and pancreatic tumors; haematological malignancies including myelodysplastic syndrome, acute myelogenous leukemia, chronic myelogenous leukemia, non Hodgkin's lymphoma, Hodgkin's disease, multiple myeloma and chronic lymphocytic leukemia; and glioma, squamous cell carcinoma of the esophagus, malignant uveal melanoma and follicular lymphoma, in a warm-blooded animal such as man.

According to a further feature of the invention, there is provided the use of a compound of formula (I), or a pharmaceutically acceptable salt thereof, as defined hereinbefore, in the manufacture of a medicament for use in the treatment of tumor-associated osteolysis, osteoporosis including ovariectomy-induced bone loss, orthopedic implant failure, autoimmune disorders including systemic lupus erythematosus, arthritis including rheumatoid arthritis, osteoarthritis, renal inflammation and glomerulonephritis; inflammatory bowel disease; transplant rejection including renal and bone marrow allografts and skin xenograft, atherosclerosis, obesity, Alzheimer's Disease and Langerhans cell histiocytosis, in a warm-blooded animal such as man.

According to a further feature of this aspect of the invention there is provided a method for producing a CSF-1R kinase inhibitory effect in a warm-blooded animal, such as man, in need of such treatment, said method comprising administering to said animal an effective amount of a compound of formula (I), or a pharmaceutically acceptable salt thereof, as defined above.

According to a further feature of this aspect of the invention there is provided a method for producing an anti-cancer effect in a warm-blooded animal, such as man, in need of such treatment, said method comprising administering to said animal an effective amount of a compound of formula (I), or a pharmaceutically acceptable salt thereof, as defined above.

According to an additional feature of this aspect of the invention there is provided a method of treating melanoma, papillary thyroid tumours, cholangiocarcinomas, colon cancer, ovarian cancer, lung cancer, leukaemias, lymphoid malignancies, carcinomas and sarcomas in the liver, kidney, bladder, prostate, breast and pancreas, and primary and recurrent solid tumours of the skin, colon, thyroid, lungs and ovaries, in a warm-blooded animal, such as man, in need of such treatment which comprises administering to said animal an effective amount of a compound of formula (I) or a pharmaceutically acceptable salt thereof as defined herein before.

According to an additional feature of the invention, there is provided a method for treating breast, ovarian, bladder, cervical, endometrial, prostate, lung, kidney and pancreatic tumors; haematological malignancies including myelodysplastic syndrome, acute myelogenous leukemia, chronic myelogenous leukemia, non Hodgkin's lymphoma, Hodgkin's disease, multiple myeloma and chronic lymphocytic leukemia; and glioma, squamous cell carcinoma of the esophagus, malignant uveal melanoma and follicular lymphoma, in a warm-blooded animal, such as man, in need of such treatment, said method comprising administering to said animal an effective amount of a compound of formula (I), or a pharmaceutically acceptable salt thereof as defined herein before.

According to an additional feature of the invention, there is provided a method for treating tumor-associated osteolysis, osteoporosis including ovariectomy-induced bone loss, orthopedic implant failure, autoimmune disorders including systemic lupus erythematosus, arthritis including rheumatoid arthritis, osteoarthritis, renal inflammation and glomerulonephritis; inflammatory bowel disease; transplant rejection including renal and bone marrow allografts and skin xenograft, atherosclerosis, obesity, Alzheimer's Disease and Langerhans cell histiocytosis, in a warm blooded animal, such as man, in need of such treatment, said method comprising administering to said animal an effective amount of a compound of formula (I), or a pharmaceutically salt thereof as defined hereinbefore.

In a further aspect of the invention there is provided a pharmaceutical composition which comprises a compound of the formula (I), or a pharmaceutically acceptable salt thereof, as defined herein before in association with a pharmaceutically-acceptable diluent or carrier for use in the production of a CSF-1R kinase inhibitory effect in a warm-blooded animal such as man.

In a further aspect of the invention there is provided a pharmaceutical composition which comprises a compound of the formula (I), or a pharmaceutically acceptable salt thereof, as defined herein before in association with a pharmaceutically-acceptable diluent or carrier for use in the production of an anti-cancer effect in a warm-blooded animal such as man.

In a further aspect of the invention there is provided a pharmaceutical composition which comprises a compound of the formula (I), or a pharmaceutically acceptable salt thereof, as defined herein before in association with a pharmaceutically-acceptable diluent or carrier for use in the treatment of melanoma, papillary thyroid tumours, cholangiocarcinomas, colon cancer, ovarian cancer, lung cancer, leukaemias, lymphoid malignancies, carcinomas and sarcomas in the liver, kidney, bladder, prostate, breast and pancreas, and primary and recurrent solid tumours of the skin, colon, thyroid, lungs and ovaries in a warm-blooded animal such as man.

In a further aspect of the invention, there is provided a pharmaceutical composition comprising a compound of formula (I), or a pharmaceutically acceptable salt thereof, as defined herein before, and at least one pharmaceutically acceptable diluent or carrier, for use in the treatment of breast, ovarian, bladder, cervical, endometrial, prostate, lung, kidney and pancreatic tumors; haematological malignancies including myelodysplastic syndrome, acute myelogenous leukemia, chronic myelogenous leukemia, non Hodgkin's lymphoma, Hodgkin's disease, multiple myeloma and chronic lymphocytic leukemia; and glioma, squamous cell carcinoma of the esophagus, malignant uveal melanoma and follicular lymphoma, in a warm-blooded animal such as man.

In a further aspect of the invention, there is provided a pharmaceutical composition comprising a compound of formula (I), or a pharmaceutically acceptable salt thereof, as defined herein before, and at least one pharmaceutically acceptable diluent or carrier, for use in the treatment of tumor-associated osteolysis, osteoporosis including ovariectomy-induced bone loss, orthopedic implant failure, autoimmune disorders including systemic lupus erythematosus, arthritis including rheumatoid arthritis, osteoarthritis, renal inflammation and glomerulonephritis; inflammatory bowel disease; transplant rejection including renal and bone marrow allografts and skin xenograft, atherosclerosis, obesity, Alzheimer's Disease and Langerhans cell histiocytosis, in a warm-blooded animal such as man.

In a further aspect of the invention, there is provided a compound of formula (I), or a pharmaceutically acceptable salt thereof, as defined herein before, for use in the production of a CSF-1R kinase inhibitory effect in a warm-blooded animal such as man.

In a further aspect of the invention, there is provided a compound of formula (I), or a pharmaceutically acceptable salt thereof, as defined herein before, for use in the production of an anti-cancer effect in a warm-blooded animal such as man.

In a further aspect of the invention, there is provided a compound of formula (I), or a pharmaceutically acceptable salt thereof, as defined herein before, for use in the treatment of breast, ovarian, bladder, cervical, endometrial, prostate, lung, kidney and pancreatic tumors; haematological malignancies including myelodysplastic syndrome, acute myelogenous leukemia, chronic myelogenous leukemia, non Hodgkin's lymphoma, Hodgkin's disease, multiple myeloma and chronic lymphocytic leukemia; and glioma, squamous cell carcinoma of the esophagus, malignant uveal melanoma and follicular lymphoma, in a warm-blooded animal such as man.

In a further aspect of the invention, there is provided a compound of formula (I), or a pharmaceutically acceptable salt thereof, as defined herein before, for use in the treatment of tumor-associated osteolysis, osteoporosis including ovariectomy-induced bone loss, orthopedic implant failure, autoimmune disorders including systemic lupus erythematosus, arthritis including rheumatoid arthritis, osteoarthritis, renal inflammation and glomerulonephritis; inflammatory bowel disease; transplant rejection including renal and bone marrow allografts and skin xenograft, atherosclerosis, obesity, Alzheimer's Disease and Langerhans cell histiocytosis, in a warm-blooded animal such as man.

According to a further aspect of the invention there is provided the use of a compound of the formula (I), or a pharmaceutically acceptable salt thereof, as defined hereinbefore in the production of a CSF-1R kinase inhibitory effect in a warm-blooded animal such as man.

According to this aspect of the invention there is provided the use of a compound of the formula (I), or a pharmaceutically acceptable salt thereof, as defined hereinbefore in the production of an anti-cancer effect in a warm-blooded animal such as man.

According to a further feature of the invention, there is provided the use of a compound of the formula (I), or a pharmaceutically acceptable salt thereof, as defined herein before in the treatment of melanoma, papillary thyroid tumours, cholangiocarcinomas, colon cancer, ovarian cancer, lung cancer, leukaemias, lymphoid malignancies, carcinomas and sarcomas in the liver, kidney, bladder, prostate, breast and pancreas, and primary and recurrent solid tumours of the skin, colon, thyroid, lungs and ovaries.

The CSF-1R kinase inhibitory treatment defined hereinbefore may be applied as a sole therapy or may involve, in addition to the compound of the invention, conventional surgery or radiotherapy or chemotherapy. Such chemotherapy may include one or more of the following categories of anti-tumour agents:—

(i) antiproliferative/antineoplastic drugs and combinations thereof, as used in medical oncology, such as alkylating agents (for example cis-platin, carboplatin, cyclophosphamide, nitrogen mustard, melphalan, chlorambucil, busulphan and nitrosoureas); antimetabolites (for example antifolates such as fluoropyrimidines like 5-fluorouracil and tegafur, raltitrexed, methotrexate, cytosine arabinoside and hydroxyurea; antitumour antibiotics (for example anthracyclines like adriamycin, bleomycin, doxorubicin, daunomycin, epirubicin, idarubicin, mitomycin-C, dactinomycin and mithramycin); antimitotic agents (for example vinca alkaloids like vincristine, vinblastine, vindesine and vinorelbine and taxoids like taxol and taxotere); and topoisomerase inhibitors (for example epipodophyllotoxins like etoposide and teniposide, amsacrine, topotecan and camptothecin);
(ii) cytostatic agents such as antioestrogens (for example tamoxifen, toremifene, raloxifene, droloxifene and iodoxyfene), oestrogen receptor down regulators (for example fulvestrant), antiandrogens (for example bicalutamide, flutamide, nilutamide and cyproterone acetate), LHRH antagonists or LHRH agonists (for example goserelin, leuprorelin and buserelin), progestogens (for example megestrol acetate), aromatase inhibitors (for example as anastrozole, letrozole, vorazole and exemestane) and inhibitors of 5α-reductase such as finasteride;
(iii) Agents which inhibit cancer cell invasion (for example metalloproteinase inhibitors like marimastat and inhibitors of urokinase plasminogen activator receptor function);
(iv) inhibitors of growth factor function, for example such inhibitors include growth factor antibodies, growth factor receptor antibodies (for example the anti-erbb2 antibody trastuzumab [Herceptin™] and the anti-erbb1 antibody cetuximab [C225]), farnesyl transferase inhibitors, MEK inhibitors, tyrosine kinase inhibitors and serine/threonine kinase inhibitors, for example inhibitors of the epidermal growth factor family (for example EGFR family tyrosine kinase inhibitors such as N-(3-chloro-4-fluorophenyl)-7-methoxy-6-(3-morpholinopropoxy)quinazolin-4-amine (gefitinib, AZD1839), N-(3-ethynylphenyl)-6,7-bis(2-methoxyethoxy)quinazolin-4-amine (erlotinib, OSI-774) and 6-acrylamido-N-(3-chloro-4-fluorophenyl)-7-(3-morpholinopropoxy)quinazolin-4-amine (CI 1033)), for example inhibitors of the platelet-derived growth factor family and for example inhibitors of the hepatocyte growth factor family;
(v) antiangiogenic agents such as those which inhibit the effects of vascular endothelial growth factor, (for example the anti-vascular endothelial cell growth factor antibody bevacizumab [Avastin™], compounds such as those disclosed in International Patent Applications WO 97/22596, WO 97/30035, WO 97/32856 and WO 98/13354) and compounds that work by other mechanisms (for example linomide, inhibitors of integrin αvβ3 function and angiostatin);
(vi) vascular damaging agents such as Combretastatin A4 and compounds disclosed in International Patent Applications WO 99/02166, WO00/40529, WO 00/41669, WO01/92224, WO02/04434 and WO02/08213;
(vii) antisense therapies, for example those which are directed to the targets listed above, such as ISIS 2503, an anti-ras antisense;
(viii) gene therapy approaches, including for example approaches to replace aberrant genes such as aberrant p53 or aberrant BRCA1 or BRCA2, GDEPT (gene-directed enzyme pro-drug therapy) approaches such as those using cytosine deaminase, thymidine kinase or a bacterial nitroreductase enzyme and approaches to increase patient tolerance to chemotherapy or radiotherapy such as multi-drug resistance gene therapy;
(ix) immunotherapy approaches, including for example ex-vivo and in-vivo approaches to increase the immunogenicity of patient tumour cells, such as transfection with cytokines such as interleukin 2, interleukin 4 or granulocyte-macrophage colony stimulating factor, approaches to decrease T-cell anergy, approaches using transfected immune cells such as cytokine-transfected dendritic cells, approaches using cytokine-transfected tumour cell lines and approaches using anti-idiotypic antibodies;
(x) Cell cycle inhibitors including for example CDK inhibitiors (eg flavopiridol) and other inhibitors of cell cycle checkpoints (eg checkpoint kinase); inhibitors of aurora kinase and other kinases involved in mitosis and cytokinesis regulation (eg mitotic kinesins); and histone deacetylase inhibitors; and
(xi) endothelin antagonists, including endothelin A antagonists, endothelin B antagonists and endothelin A and B antagonists; for example ZD4054 and ZD1611 (WO 96 40681), atrasentan and YM598.

Such conjoint treatment may be achieved by way of the simultaneous, sequential or separate dosing of the individual components of the treatment. Such combination products employ the compounds of this invention within the dosage range described hereinbefore and the other pharmaceutically-active agent within its approved dosage range.

In addition to their use in therapeutic medicine, the compounds of formula (I) and their pharmaceutically acceptable salts are also useful as pharmacological tools in the development and standardisation of in vitro and in vivo test systems for the evaluation of the effects of inhibitors of CSF-1R kinase in laboratory animals such as cats, dogs, rabbits, monkeys, rats and mice, as part of the search for new therapeutic agents.

In the above other pharmaceutical composition, process, method, use and medicament manufacture features, the alternative and preferred embodiments of the compounds of the invention described herein also apply.

### EXAMPLES

The invention will now be illustrated by the following non limiting examples in which, unless stated otherwise:

(i) temperatures are given in degrees Celsius (° C.); operations were carried out at room or ambient temperature, that is, at a temperature in the range of 18-25° C.;
(ii) organic solutions were dried over anhydrous sodium sulphate; evaporation of solvent was carried out using a rotary evaporator under reduced pressure (600-4000 Pascals; 4.5-30 mmHg) with a bath temperature of up to 60° C.;
(iii) in general, the course of reactions was followed by TLC and reaction times are given for illustration only;
(iv) final products had satisfactory proton nuclear magnetic resonance (NMR) spectra and/or mass spectral data;
(v) yields are given for illustration only and are not necessarily those which can be obtained by diligent process development; preparations were repeated if more material was required;
(vii) when given, NMR data is in the form of delta values for major diagnostic protons, given in parts per million (ppm) relative to tetramethylsilane (TMS) as an internal standard, determined at 400 MHz using perdeuterio dimethyl sulphoxide (DMSO-d₆) as solvent unless otherwise indicated;
(vii) chemical symbols have their usual meanings; SI units and symbols are used;
(viii) solvent ratios are given in volume:volume (v/v) terms; and
(ix) mass spectra were run with an electron energy of 70 electron volts in the chemical ionization (CI) mode using a direct exposure probe; where indicated ionization was effected by electron impact (EI), fast atom bombardment (FAB) or electrospray (ESP); values for m/z are given; generally, only ions which indicate the parent mass are reported; and unless otherwise stated, the mass ion quoted is (MH)⁺;
(x) where a synthesis is described as being analogous to that described in a previous example the amounts used are the millimolar ratio equivalents to those used in the previous example;
(xi) the following abbreviations have been used:

HATU O-(7-Azabenzotriazol-1-yl)-N,N,N′,N′-tetramethyluronium hexafluorophosphate;

THF tetrahydrofuran;

DMF N,N-dimethylformamide;

EtOAc ethyl acetate;

DIEA N,N-diisopropylethylamine;

DCM dichloromethane;

DMSO dimethylsulphoxide;

MeCN acetonitrile;

MeOH methanol; and

DPPA Diphenylphosphoiyl azide

(xii) “ISCO” refers to normal phase flash column chromatography using 12 g and 40 g pre-packed silica gel cartridges used according to the manufacturers instruction obtained from ISCO, Inc, 4700 superior street Lincoln, Nebr., USA.; and
(xiii) “Gilson HPLC” refers to a YMC-AQC18 reverse phase HPLC Column with dimension 20 mm/100 and 50 mm/250 in water/MeCN with 0.1% TFA as mobile phase, obtained
(xiv) Parr Hydrogenator or Parr shaker type hydrogenators are systems for treating chemicals with hydrogen in the presence of a catalyst at pressures up to 5 atmospheres (60 psig) and temperatures to 80° C.

### Preparation of the Starting Materials

### Method 1

### 1,3-Thiazol-5-amine

To a solution of 1,3-thiazole-5-carboxylic acid (728 mg, 5.6 mmol) in tert-BuOH (19 mL) was added Et₃N (2.4 mL, 17 mmol) and DPPA (2.5 mL, 11.3 mmol) and the resulting dark red solution was heated to reflux for 8 hours. After cooling, EtOAc was added, and the organic layer washed with saturated NaHCO₃ solution, water, brine, and dried (MgSO₄). Evaporation of the solvents under reduced pressure afforded tert-butyl 1,3-thiazol-5-ylcarbamate (500 mg), which was used in the next step without any further purification. m/z: 201.

To a solution of tert-butyl 1,3-thiazol-5-ylcarbamate (500 mg) in MeOH (10 mL) at 0° C. was added slowly a solution of 4N HCl in dioxane (5 ml) and the resulting yellow solution was stirred at room temperature for 2 hours. The title compound was isolated as a pale yellow solid after filtration (150 mg) as its hydrochloride salt. m/z: 101.

### Method 2

### 2-Methyl-1,3-thiazol-5-amine

To a solution of aminoacetonitrile bisulfate (6.4 g, 41.6 mmol) in anhydrous MeOH (75 ml) at 0° C. was added Et₃N (11.6 mL, 83 mmol). After 30 minutes, ethyl dithioacetate (5 g, 41.6 mmol) was added and the resulting dark orange solution stirred at room temperature for 2 hours. Half the solvent was removed under reduced pressure. The solution was diluted with an equivalent volume of EtOAc, washed with water, and dried (Na₂SO₄). The solvents were removed under reduced pressure and the residue slurried in warm EtOAc, cooled in an ice bath, and filtered to give 2.45 g (52%) of a brown solid.

¹H NMR DMSO-d6: 6.62 (s, 1H) 5.36 (bs, 2H) 2.89 (s, 3H); m/z: 115.

### Method 3

### 5-Amino-N-methyl-1,3-thiazole-2-carboxamide

To a solution of 2-chloro-N-methylacetamide (1.0 g, 9.3 mmol) in DMF (10 ml) was added Et₃N (2.9 mL) and sulfur (595 mg, 18.6 mmol). After 2 hours, methyl iodide (0.6 mL, 10.2 mmol) was added and the dark solution was stirred at room temperature for a further 3 hours. The reaction mixture was partitioned between EtOAc and water, and the organic layer washed with 1N sodium thiosulfate solution, water, and dried (MgSO₄). Evaporation of the solvents afforded methyl 3-(methylamino)-3-oxoethane(dithioate) (300 mg), which was used without further purification in the next step.

To a solution of aminoacetonitrile bisulfate (400 mg) in EtOAc (10 ml) was added Et₃N (5 mL) and methyl 3-(methylamino)-3-oxoethane(dithioate) (300 mg), and the resulting dark orange solution was stirred at room temperature for 18 hours. The title compound was isolated via filtration (50 mg). m/z: 158.

### Methods 4 and 5

The following compounds were prepared by a procedure analogous to that of Method 3, using the appropriate starting material.

| Method | Compound | m/z | Starting Material |
|---|---|---|---|
| 4 | 2-(Morpholin-4- | 206 | 4-(Chloroacetyl)morpholine |
| | ylcarbonyl)-1,3-thiazol-5- | | |
| | amine | | |
| 5 | 5-Amino-N-methoxy-N- | 188 | 2-Chloro-N-methoxy-N- |
| | methyl-1,3-thiazole-2- | | methylacetamide |
| | carboxamide | | |

### Method 6

### 3-(1-Cyano-1-methylethyl)benzoic acid

A solution of 3-(1-cyano-1-methylethyl)benzoic acid methyl ester (Method 14, 5.5 g, 27.1 mmol) in 100 ml of THF/MeOH/water (3:1:1) was treated with lithium hydroxide (1.95 g) in 20 ml water. The mixture was stirred at room temperature for 12 hours. The solvent was removed under reduced pressure and the resulting solution was diluted with water, then acidified with 10% HCl to pH 2. The resulting white solid (4.83 g, 94%) was filtered, washed with water and dried.

¹H NMR: 13.00 (s, 1H), 7.95 (s, 1H), 7.80 (d, 1H), 7.65 (d, 1H), 7.45 (m, 1H), 1.60 (s, 6H); m/z: 189.

### Methods 7 to 13

The following compounds were prepared by a procedure analogous to that of Method 6, using the appropriate starting material.

| Method | Compound | m/z | Starting Material |
|---|---|---|---|
| 7 | 5-{[3-(1-Cyano-1- | 321 | Methyl 5-{[3-(1-cyano-1- |
| | methylethyl)benzoyl]amino}-2- | | methylethyl)benzoyl]amino}-2- |
| | methylbenzoic acid | | methylbenzoate |
| | | | (Method 16) |
| 8 | 2-Chloro-5-{[3- | 342 | Methyl 2-chloro-5-{[3- |
| | (trifluoromethyl)benzoyl]amino}- | | (trifluoromethyl)benzoyl]- |
| | benzoic acid | | amino}benzoate |
| | | | (Method 21) |
| 9 | 2-Chloro-5-[(3- | 308 | Methyl 2-chloro-5-[(3- |
| | chlorobenzoyl)amino]benzoic | | chlorobenzoyl)amino]benzoate |
| | acid | | (Method 17) |
| 10 | 2-Chloro-5-[(3,5- | 302 | Methyl 2-chloro-5-[(3,5- |
| | dimethylbenzoyl)amino]benzoic acid | | dimethylbenzoyl)amino]benzoate |
| | | | (Method 18) |
| 11 | 2-Methyl-5-{[3- | 322 | Methyl 2-methyl-5-{[3- |
| | (trifluoromethyl)benzoyl]amino}- | | (trifluoromethyl)benzoyl]amino}- |
| | benzoic acid | | benzoate |
| | | | (Method 20) |
| 12 | 2-Chloro-5-{[3-fluoro-5- | 360 | Methyl 2-chloro-5-{[3-fluoro-5- |
| | (trifluoromethyl)benzoyl]amino}- | | (trifluoromethyl)benzoyl]amino}- |
| | benzoic acid | | benzoate |
| | | | (Method 19) |
| 13 | Cyclohexyl (difluoro) acetic acid | 177 | Ethyl cyclohexyl(difluoro)acetate |
| | | | (Method 36) |

### Method 14

### 3-(1-Cyano-1-methylethyl)benzoic acid methyl ester

A solution of 3-cyanomethyl-benzoic acid methyl ester (Method 15, 7.2 g, 41.1 mmol) in anhydrous DMSO (80 ml) was treated with NaH (60% in mineral oil, 4.9 g, 123.3 mmol). Methyl iodide was added dropwise at 0° C. The reaction mixture was stirred at room temperature for 12 hours, quenched with water (200 ml) and extracted with EtOAc. The combined organics were dried and concentrated under reduced pressure. The crude product was purified by column chromatography utilizing an ISCO system (hexane-EtOAc) to give 5.5 g (66%) of a colourless oil.

¹H NMR: 8.05 (s, 1H), 7.90 (d, 1H), 7.75 (d, 1H), 7.55 (m, 1H), 3.80 (s, 3H), 1.62 (s, 6H); m/z: 203.

### Method 15

### 3-Cyanomethyl-benzoic acid methyl ester

A suspension of methyl-3-(bromomethyl)benzoate (13.5 g, 58.9 mmol) and sodium cyanide (4.33 g, 88.4 mmol) in DMF (25 ml) and water (1 ml) was stirred at 75° C. for 5 hours. The reaction mixture was quenched with water (50 ml), extracted with EtOAc (3×100 ml) and the combined organics were dried and concentrated under reduced pressure. The residue was purified by column chromatography utilizing an ISCO system (hexane-EtOAc) to give 7.2 g (70%) of a colourless oil.

¹H NMR: 7.90 (s, 1H), 7.86 (d, 1H), 7.60 (d, 1H), 7.50 (m, 1H), 4.10 (s, 2H), 3.80 (s, 3H); m/z: 175.

### Method 16

### Methyl 5-{[3-(1-cyano-1-methylethyl)benzoyl]amino}-2-methylbenzoate

A solution of methyl 5-amino-2-methylbenzoate (Method 22, 2.7 g, 16.4 mmol), 3-(1-cyano-1-methylethyl)benzoic acid (Method 6, 3.13 g, 16.6 mmol) and N,N-diisopropylethylamine (8.67 ml, 49.8 mmol) in DMF (33 ml) at 0° C. was treated with HATU (9.47 g, 24.9 mmol). The reaction was stirred at room temperature for 24 hours, quenched with water (30 ml) and extracted with EtOAc (100 ml). The organic layer was washed with brine (200 ml), dried (MgSO₄) and concentrated under reduced pressure to give 5.58 g of a reddish-brown oil. m/z: 336.

### Methods 17 and 18

The following compounds were prepared by a procedure analogous to that of Method 16, using the appropriate starting material and methyl 5-amino-2-chlorobenzoate Method 24.

| Method | Compound | m/z | Starting Material |
|---|---|---|---|
| 17 | Methyl 2-chloro-5-[(3- | 324 | 3-Chlorobenzoic |
| | chlorobenzoyl)amino]benzoate | | acid |
| 18 | Methyl 2-chloro-5-[(3,5- | 318 | 3,5- |
| | dimethylbenzoyl)amino]benzoate | | Dimethylbenzoic |
| | | | acid |

### Method 19

### Methyl 2-chloro-5-{[3-fluoro-5-(trifluoromethyl)benzoyl]amino}benzoate

To a solution of methyl 5-amino-2-chlorobenzoate (Method 24, 2.25 g, 12.1 mmol) and triethylamine (2.53 ml, 18.2 mmol) in DCM (15 ml) at 0° C. was added 3-fluoro-5-(trifluoromethyl)benzoyl chloride (3.02 g, 13.3 mmol). After 1.5 hours, the reaction mixture was diluted with DCM (100 ml), washed with 1N HCl (30 ml), water (30 ml), brine (30 ml) and dried (MgSO₄). The crude product was recrystallized from EtOAc:Hex (3 crops) to give 3.55 g (78%) white solid.

¹H NMR CDCl₃ 8.05 (s, 1H), 7.86 (m, 3H), 7.79 (d, 1H), 7.55 (d, 1H), 7.48 (d, 1H), 3.94 (s, 3H); m/z: 374.

### Methods 20 and 21

The following compounds were prepared by a procedure analogous to that of Method 19, using the appropriate starting material and 3-(trifluoromethyl)benzoyl chloride.

| Method | Compound | m/z | Starting Material |
|---|---|---|---|
| 20 | Methyl 2-methyl-5-{[3- | 338 | Methyl 5-amino-2- |
| | (trifluoromethyl)benzoyl]amino}- | | methylbenzoate |
| | benzoate | | (Method 22) |
| 21 | Methyl 2-chloro-5-{[3- | 358 | Methyl 5-amino-2- |
| | (trifluoromethyl)benzoyl]amino}- | | chlorobenzoate |
| | benzoate | | (Method 24) |

### Method 22

### Methyl 5-amino-2-methylbenzoate

A solution of methyl 2-methyl-5-nitrobenzoate (Method 23; 3.4 g) and 10% palladium on carbon (672 mg) in MeOH (20 ml) was treated with H₂ for 48 hours. The reaction mixture was then filtered through diatomaceous earth and washed with MeOH (20 ml) and EtOAc (10 ml). The solvents were removed under reduced pressure to give 2.7 g of a brown oil.

¹H NMR: 7.11 (d, 1H), 6.94 (d, 1H), 6.69 (dd, 1H), 5.13 (s, 2H), 3.78 (s, 3H), 2.33 (s, 3H); m/z: 165.

### Method 23

### Methyl 2-methyl-5-nitrobenzoate

A solution of 2-methyl-5-nitrobenzoic acid (3.9 g, 21.5 mmol) in MeOH (20 ml) was treated with HCl gas for 10 min. The reaction was then refluxed in a sealed tube at 65° C. for 24 hours. The solvent was evaporated giving a cream coloured solid (4.8 g), which was dissolved in EtOAc (200 ml), washed with water (200 ml), brine (200 ml), and dried (MgSO₄). The solvents were removed under reduced pressure to give 3.4 g of a white solid.

¹H NMR: 8.48 (d, 1H), 8.27 (dd, 1H), 7.60 (d, 1H), 3.87 (s, 3H), 2.60 (s, 3H); m/z: 196.

### Method 24

### Methyl 5-amino-2-chlorobenzoate

Thionyl chloride (1.30 ml, 17.8 mmol) was added to a solution of 5-amino-2-chlorobenzoic acid (3.06 g, 17.8 mmol) in MeOH (20 ml). The reaction mixture was stirred for 16 hours, concentrated and the residue dissolved in EtOAc (150 ml). The organic layer was washed with sat. NaHCO₃ solution (75 ml), water (50 ml), brine (50 ml) and dried (MgSO₄). The solvents were removed under reduced pressure to give 2.25 g (68%) of a colorless oil.

¹H NMR CDCl₃ 7.18 (d, 1H), 7.11 (d, 1H), 6.71 (dd, 1H), 3.90 (s, 3H); m/z: 186.

### Method 25

### 5-Amino-2-methyl-N-(2-methyl-1,3-thiazol-5-yl)benzamide

A solution of tert-butyl (4-methyl-3-{[(2-methyl-1,3-thiazol-5-yl)amino]carbonyl}phenyl)carbamate (Example 84, 3.4 g, 9.79 mmol) in MeOH was treated with HCl gas for 30 minutes. The reaction mixture was stirred for 20 hours, and concentrated. The crude product was recrystallized from MeOH to give 1.8 g (74%) of a white solid. m/z: 248.

### Method 26

The following compound was prepared by a procedure analogous to that of Method 25, using the appropriate starting material.

| Method | Compound | m/z | Starting Material |
|---|---|---|---|
| 26 | 5-amino-2-chloro-N- | 267 | tert-Butyl (4-chloro-3-{[(2- |
| | (2-methyl-1,3-thiazol-5- | | methyl-1,3-thiazol-5- |
| | yl)benzamide | | yl)amino]carbonyl}- |
| | | | phenyl)carbamate |
| | | | (Example 85) |

### Method 27

### 5-[(tert-Butoxycarbonyl)amino]-2-methylbenzoic acid

A solution of methyl 5-[(tert-butoxycarbonyl)amino]-2-methylbenzoate (Method 29, 14.8 g, 55.9 mmol) in MeOH:THF:water (1:1:1, 300 ml) was treated with KOH (5 eq.) and stirred for 20 hours. The organic solvent was removed under reduced pressure, and the remaining aqueous phase was acidified to pH=4 with dilute HCl. The aqueous phase was extracted with EtOAc and the organic layer dried ((Na₂SO₄) and concentrated to give 12.1 g (86%) of a white solid.

¹H NMR: 9.28 (s, 1H), 7.80 (s, 1H), 7.36 (dd, 1H), 7.04 (d, 1H), 2.37 (s, 3H), 1.45 (s, 9H).

### Method 28

The following compound was prepared by a procedure analogous to that of Method 27, using the appropriate starting material.

| Method | Compound | m/z | Starting Material |
|---|---|---|---|
| 28 | 5-[(tert- | 269 | Methyl 5-[(tert- |
| | butoxycarbonyl)amino]-2- | | butoxycarbonyl)amino]-2- |
| | chlorobenzoic acid | | chlorobenzoate |
| | | | (Method 30) |

### Method 29

### Methyl 5-[(tert-butoxycarbonyl)amino]-2-methylbenzoate

To a solution of methyl 5-amino-2-methylbenzoate (Method 22, 4.3 g, 26.0 mmol) in THF (160 ml) and water (40 ml) was added di-tert-butyldicarbonate (17.0 g, 78.1 mmol) and K₂CO₃ (10.8 g, 78.1 mmol). The reaction mixture was stirred for 16 hours, the organic solvent was removed under reduced pressure, and the remaining aqueous phase was extracted with EtOAc. After concentration of the organic layer, chromatography gave 6.2 g (90%) of a white solid.

¹H NMR: 9.46 (s, 1H), 8.05 (d, 1H), 7.47 (dd, 1H), 7.19 (d, 1H), 3.81 (s, 3H), 2.42 (s, 3H), 1.47 (s, 9H).

### Method 30

The following compound was prepared by a procedure analogous to that of Method 29, using the appropriate starting material.

| Method | Compound | m/z | Starting Material |
|---|---|---|---|
| 30 | Methyl 5-[(tert- | 285 | Methyl 5-amino-2- |
| | butoxycarbonyl)amino]-2- | | chlorobenzoate |
| | chlorobenzoate | | (Method 24) |

### Method 31

### 3-Cyclopropyl-5-fluorobenzoic acid

To a solution of 3-bromo-5-fluorobenzoic acid (0.500 g, 4.56 mmol) and cyclopropylboronic acid (0.590 g, 6.84 mmol) in toluene (15 ml) and water (0.75 ml) was added K₃PO₄ (3.86 g, 18.24 mmol) and Pd(PPh₃)₄ (1.05 g, 0.912 mmol). The reaction mixture was heated to 100° C. for 12 hours, cooled to room temperature, and quenched with 10% aqueous NaOH (100 ml). The reaction mixture was washed with EtOAc (100 ml) and the resulting aqueous layer isolated and brought to a pH of ˜2 by the careful addition of 3N HCl. The resulting precipitate was filtered, washed with water (100 ml), and dried under vacuum for 24 hours to give 0.31 g (37%) off-white solid; m/z: 181.

### Method 32

### 3-Cyclopropylbenzoic acid

To a solution of diethyl zinc (12.3 ml, 1 M in hexanes) in DCM (20 ml) at 0° C. was added dropwise via syringe trifluoroacetic acid (1.40 g, 12.3 mmol), and after 20 minutes stirring, diiodomethane (3.30 g, 12.3 mmol). After 20 minutes, methyl 3-vinylbenzoate (1.00 g, 6.16 mmol) was added, and the cooling bath removed. After 3 hours, the reaction was quenched by the addition of saturated NH₄Cl solution (50 ml). The aqueous phase was extracted with DCM (3×50 ml), and the combined organic extract dried (MgSO₄) and concentrated in vacuo to yield the crude reaction product which was purified by column chromatography (hexanes/EtOAc 10:1) to give 1.01 g (94%) methyl 3-cyclopropylbenzoate as a colourless oil; m/z: 177.

To a solution of methyl-3-cyclopropylbeizoate (0.275 g, 1.56 mmol) in MeOH (10 ml) and H₂O (1 ml) was added LiOH.H₂O (0.131 g, 3.00 mmol). After 3 hours the pH was adjusted to 3 by the addition of 3N HCl, and the aqueous phase extracted with EtOAc (3×25 ml). The combined organic extract was washed with brine (25 mL), dried (MgSO₄), and concentrated in vacuo to yield 0.192 g (76%) white solid; m/z: 161.

### Method 33

### 2-Isopropyl-1,3-thiazol-5-amine

A solution of 2-methylpropanoic acid (2.5 g, 28.8 mmol) in 1,2,4-trichlorobenzene (5 mL) was added to a suspension of 2,4-bis(methylthio)-1,3,2,4-dithiadiphosphetane 2,4-disulfide (Davy Reagent) (5 g, 15.85 mmol) in 1,2,4-trichlorobeizene (20 ml) at room temperature. The resulting yellow reaction mixture was heated to 130° C. for 10 min. The crude methyl 2-methylpropane dithioate was collected with 1,2,4-trichlorobenzene via vacuum distillation, which was used in the next step without any further purification. Aminoacetonitrile (4.43 g, 28.8 mmol) in 40 ml of methanol was treated with TEA (5.8 g, 57.6 mmol). The reaction was then cooled to 0° C. and methyl 2-methylpropane dithioate (˜28 mmol) in 1,2,4-trichlorobenzene was added to the reaction with an addition funnel over 15 minutes. The resulting reaction mixture was allowed to stir to room temperature over 2 days before being concentrated in vacuo. The residue was partitioned between water and chloroform, separated, and the aqueous phase was extracted an additional with CHCl₃. The organic layers were combined, dried over anhydrous sodium sulfate, filtered, the filtrate was concentrated in vacuo giving the crude product. The residue was purified on 120 g of SiO₂ using hexanes:EtOAc (1:1) as eluent yielding 0.620 g (15% over two steps) of the title compound as a brown solid. ¹H NMR (400 MHz, DMSO): 6.55 (s, 1H), 5.30 (s, 2H), 3.00 (m, 1H), 1.20 (d, 6H); m/z: 142.

### Method 34

The following compound was prepared by the procedure of Method 33, using the appropriate starting material.

| Method | Compound | ¹H NMR (300 MHz) | m/z | Starting Material |
|---|---|---|---|---|
| 34 | 2-Cyclopropyl-1,3- | DMSO-d6 6.50 (s, | 140 | Cyclopropanecarboxylic |
| | thiazol-5-amine | 1H), 5.30 (s, br, 2H), | | acid |
| | | 2.05 (m, 1H), | | |
| | | 0.91 (m, 2H), 0.76 (m, 2H) | | |

### Method 35

### Ethyl difluoro (2-iodocyclohexyl)acetate

Cyclohexene (1.64 g, 20 mmol) and ethyl iododifluoroacetate (5 g, 20 mmol) were dissolved in a solvent system of water (20 ml) and acetonitrile (20 ml). Sodium dithionite (7.4 g) and sodium bicarbonate (3.7 g) were then added to the solution. The mixture was allowed to stir at ambient temperature for 12 h. The reaction was then treated with water (100 ml), poured into a separatory funnel, and extracted with ether (3×40 ml). The combined organic layer was washed with saturated aqueous NaCl, dried over anhydrous sodium sulfate, and concentrated in vacuo to yield the crude product which was purified via SiO₂ chromatography using hexane-EtOAc (9:1) as eluent to give 4.9 g (74%) of the title compound as a mixture of diastereoisomers.

### Method 36

### Ethyl cyclohexyl (difluoro) acetate

A flask fitted with a stir bar and a condenser topped with a nitrogen inlet was charged with Zinc(s) (1.92 g, 29.5 mmol), of NiCl₂.6H₂O (0.354 g, 1.476 mmol), 2.5 drops of water, and 25 ml of THF. The resulting mixture was stirred at 25° C. for 15 min, and then ethyl difluoro (2-iodocyclohexyl)acetate (Method 35) (4.9 g, 14.76 mmol) was added and the reaction was stirred for 4 h. The reaction mixture was then poured into a saturated aqueous solution of NH₄Cl and extracted with ether (3×30 ml). The combined organic phase was dried with MgSO₄, filtered, and concentrated in vacuo to yield the crude product which was purified via SiO₂ chromatography using hexane-EtOAc (9:1) as eluent to give 1.5 g (49%) of the title compound as a light yellow oil.

### Example 1

### 5-{[3-(1-Cyano-1-methylethyl)benzoyl]amino}-2-methyl-N-(2-methyl-1,3-thiazol-5-yl)benzamide

A solution of 5-{[3-(1-cyano-1-methylethyl)benzoyl]amino}-3-2-methylbenzoic acid (Method 7, 82 mg, 0.25 mmol), 2-methyl-1,3-thiazol-5-amine (Method 2, 28 mg, 0.25 mmol), HATU (101 mg, 0.275 mmol) and N,N-diisopropylethylamine (0.135 mL) in DMF (0.5 mL) was stirred for 16 hours at room temperature. The reaction mixture was partitioned between water and EtOAc, and the organic layer was washed with brine and dried (MgSO₄). Purification by reverse HPLC (5%-95% water-MeCN, 15 minutes) afforded 51 mg (48%) of title compound after evaporation of the solvents.

¹H NMR CDCl₃ 11.80 (s, 1H) 10.39 (s, 1H) 8.64 (s, 1H) 7.90-8.02 (m, 3H) 7.78-7.81 (m, 2H) 7.60 (t, 1H) 7.35 (d, 1H) 2.49 (s, 3H) 2.39 (s, 3H) 1.76 (s, 6H); m/z 419.

### Examples 2-24

The following compounds were prepared by a procedure analogous to that described in Example 1 using 1,3-thiazol-5-amine (Method 1), 2-methyl-1,3-thiazol-5-amine (Method 2), 5-amino-N-methyl-1,3-thiazole-2-carboxamide (Method 3), 2-(morpholin-4-ylcarbonyl)-1,3-thiazol-5-amine (Method 4), 5-amino-N-methoxy-N-methyl-1,3-thiazole-2-carboxamide (Method 5), 2-Isopropyl-1,3-thiazol-5-amine (Method 33), or 2-Cyclopropyl-1,3-thiazol-5-amine (Method 34), and the appropriate starting material. In some cases, alternative methods of purification were required (column chromatography or recrystallization from EtOAc:Hex).

| Ex. | Compound | ¹H NMR (300 MHz) | m/z | Starting Material |
|---|---|---|---|---|
| 2 | 2-Chloro-N-1,3-thiazol-5- | DMSO-d6 12.06 (s, 1H) | 426 | 2-Chloro-5-{[3- |
| | yl-5-{[3- | 10.75 (s, 1H) 8.69 (s, 1H) | | (trifluoromethyl)benzoyl]- |
| | (trifluoromethyl)benzoyl]- | 8.32 (s, 1H) 8.29 (d, | | amino}benzoic |
| | amino}benzamide | 1H) 8.06 (s, 1H) | | acid |
| | | 7.99 (m, 2H) 7.82 (m, 1H) | | (Method 8) |
| | | 7.71 (s, 1H) 7.64 (d, 1H) | | |
| 3 | 2-Chloro-5-[(3- | CD₃OD 8.63 (s, 1H) | 392 | 2-Chloro-5-[(3- |
| | chlorobenzoyl)amino]-N- | 8.03 (d, 1H) 7.96 (m, 1H) | | chlorobenzoyl)amino]- |
| | 1,3-thiazol-5-ylbenzamide | 7.86 (m, 2H) 7.71 (s, | | benzoic acid |
| | | 1H) 7.60 (d, 1H) | | (Method 9) |
| | | 7.52 (m, 2H) | | |
| 4 | 2-Chloro-5-[(3,5- | CD₃OD 8.63 (s, 1H) | 386 | 2-Chloro-5-[(3,5- |
| | dimethylbenzoyl)amino]- | 8.03 (s, 1H) 7.85 (d, 1H) | | dimethylbenzoyl)amino]- |
| | N-1,3-thiazol-5- | 7.71 (s, 1H) 7.53 (m, | | benzoic acid |
| | ylbenzamide | 3H) 7.24 (s, 1H) 2.38 (s, | | (Method 10) |
| | | 6H) | | |
| 5 | 5-{[3-(1-Cyano-1- | DMSO-d6 11.83 (s, 1H) | 406 | 5-{[3-(1-Cyano-1- |
| | methylethyl)benzoyl]amino}- | 10.45 (s, 1H) 8.66 (s, 1H) | | methylethyl)benzoyl]- |
| | 2-methyl-N-1,3- | 8.01-8.11 (m, 1H) | | amino}-2- |
| | thiazol-5-ylbenzamide | 7.91-8.01 (m, 2H) | | methylbenzoic acid |
| | | 7.85 (dd, 1H) 7.73-7.81 (m, 1H) | | (Method 7) |
| | | 7.72 (s, 1H) 7.62 (t, 1H) | | |
| | | 7.35 (d, 1H) 2.39 (s, | | |
| | | 3H) 1.76 (s, 6H) | | |
| 6 | 2-Methyl-N-(2-methyl- | CDCl₃ 11.70 (s, 1H) | 421 | 2-Methyl-5-{[3- |
| | 1,3-thiazol-5-yl)-5-{[3- | 10.41 (s, 1H) 8.51 (s, 1H) | | (trifluoromethyl)benzoyl]- |
| | (trifluoromethyl)benzoyl]- | 7.89-8.01 (m, 2H) | | amino}benzoic |
| | amino}benzamide | 7.77-7.81 (m, 3H) | | acid |
| | | 7.60 (t, 1H) 7.35 (d, 1H) | | (Method 11) |
| | | 2.48 (s, 3H) 1.77 (s, 3H) | | |
| 7 | 2-Chloro-5-[(3- | DMSO-d6 11.84 (s, 1H) | 406 | 2-Chloro-5-[(3- |
| | chlorobenzoyl)amino]-N- | 10.61 (s, 1H) 8.02 (s, 2H) | | chlorobenzoyl)amino]- |
| | (2-methyl-1,3-thiazol-5- | 7.92 (m, 2H) 7.69 (d, | | benzoic acid |
| | yl)benzamide | 1H) 7.59 (m, 2H) | | (Method 9) |
| | | 7.40 (s, 1H) 2.57 (s, 3H) | | |
| 8 | 2-Chloro-5-[(3,5- | DMSO-d6 11.83 (s, 1H) | 400 | 2-Chloro-5-[(3,5- |
| | dimethylbenzoyl)amino]- | 10.43 (s, 1H) 8.02 (s, 1H) | | dimethylbenzoyl)amino]- |
| | N-(2-methyl-1,3-thiazol- | 7.96 (d, 1H) 7.57 (m, | | benzoic acid |
| | 5-yl)benzamide | 3H) 7.40 (s, 1H) 7.24 (s, | | (Method 10) |
| | | 1H) 2.57 (s, 3H) 2.35 (s, | | |
| | | 6H) | | |
| 9 | 2-Chloro-N-(2-methyl- | DMSO-d6 11.85 (s, 1H) | 440 | 2-Chloro-5-{[3- |
| | 1,3-thiazol-5-yl)-5-{[3- | 10.72 (s, 1H) 8.30 (s, 1H) | | (trifluoromethyl)benzoyl]- |
| | (trifluoromethyl)benzoyl]- | 8.25 (d, 1H) 8.02 (s, | | amino}benzoic |
| | amino}benzamide | 1H) 7.96 (m, 2H) | | acid |
| | | 7.80 (m, 1H) 7.61 (d, 1H) | | (Method 8) |
| | | 7.41 (s, 1H) 2.57 (s, 3H) | | |
| 10 | 2-Chloro-5-{[3-fluoro-5- | DMSO-d6 11.87 (s, 1H) | 458 | 2-Chloro-5-{[3- |
| | (trifluoromethyl)benzoyl]- | 10.76 (s, 1H) 8.18 (s, 1H) | | fluoro-5- |
| | amino}-N-(2-methyl-1,3- | 8.13 (d, 1H) 8.00 (m, | | (trifluoromethyl)benzoyl]- |
| | thiazol-5-yl)benzamide | 3H) 7.62 (d, 1H) | | amino}benzoic |
| | | 7.41 (s, 1H) 2.57 (s, 3H) | | acid |
| | | | | (Method 12) |
| 11 | 5-[(5-{[3-(1-Cyano-1- | DMSO-d6 11.80 (s, 1H) | 463 | 5-{[3-(1-Cyano-1- |
| | methylethyl)benzoyl]amino}- | 10.21 (s, 1H) 8.06 (d, 1H) | | methylethyl)benzoyl]- |
| | 2- | 7.83-7.89 (m, 1H) | | amino}-2- |
| | methylbenzoyl)amino]-N- | 7.74-7.80 (m, 1H) | | methylbenzoic acid |
| | methyl-1,3-thiazole-2- | 7.70 (dd, 1H) 7.51-7.62 (m, 2H) | | (Method 7) |
| | carboxamide | 7.42 (t, 1H) 7.11 (d, 1H) | | |
| | | 2.57 (s, 3H) 2.56 (s, 3H) | | |
| | | 1.72 (s, 6H) | | |
| 12 | 2-Chloro-5-[(3,5- | DMSO-D6 d ppm | 499 | 2-Chloro-5-[(3,5- |
| | dimethylbenzoyl)amino]- | 12.34 (s, 1H) 10.47 (s, 1H) | | dimethylbenzoyl)amino]- |
| | N-[2-(morpholin-4- | 8.09 (d, 1H) 7.95 (dd, 1H) | | benzoic acid |
| | ylcarbonyl)-1,3-thiazol-5- | 7.74 (s, 1H) | | (Method 10) |
| | yl]benzamide | 7.50-7.61 (m, 3H) 7.24 (s, 1H) | | |
| | | 3.66 (m, 8H) 2.35 (s, | | |
| | | 6H) | | |
| 13 | 5-[(2-Chloro-5-{[3- | DMSO-D6 12.39 (s, 1H) | 538 | 2-Chloro-5-{[3- |
| | (trifluoromethyl)benzoyl]- | 10.75 (s, 1H) | | (trifluoromethyl)benzoyl]- |
| | amino}benzoyl)amino]-N- | 8.23-8.32 (m, 2H) 8.08 (d, 1H) | | amino}benzoic |
| | methoxy-N-methyl-1,3- | 7.93-8.02 (m, 2H) | | acid |
| | thiazole-2-carboxamide | 7.77-7.84 (m, 2H) 7.63 (d, 1H) | | (Method 8) |
| | | 3.80 (s, 3H) 3.48 (s, 3H) | | |
| 14 | 5-({2-Chloro-5-[(3,5- | | 473 | 2-Chloro-5-[(3,5- |
| | dimethylbenzoyl)amino]benzoyl}- | | | dimethylbenzoyl)amino]- |
| | amino)-N- | | | benzoic acid |
| | methoxy-N-methyl-1,3- | | | (Method 10) |
| | thiazole-2-carboxamide | | | |
| 15 | 5-[(5-{[3-(1-Cyano-1- | | 492 | 5-{[3-(1-Cyano-1- |
| | methylethyl)benzoyl]amino}- | | | methylethyl)benzoyl]- |
| | 2- | | | amino}-2- |
| | methylbenzoyl)amino]-N- | | | methylbenzoic acid |
| | methoxy-N-methyl-1,3- | | | (Method 7) |
| | thiazole-2-carboxamide | | | |
| 16 | 2-Chloro-N-(2-isopropyl- | DMSO-d6 11.95 (s, 1H), | 468 | 2-Chloro-5-{[3- |
| | 1,3-thiazol-5-yl)-{[3- | 10.80 (s, 1H), 8.30 (m, | | (trifluoromethyl)benzoyl]- |
| | (trifluoromethyl)benzoyl]- | 2H), 8.00 (m, 3H), | | amino}benzoic |
| | amino}benzamide | 7.80 (m, 1H), 7.55 (d, 1H), | | acid |
| | | 7.50 (s, 1H), 3.25 (m, | | (Method 8) |
| | | 1H), 1.30 (d, 6H) | | |
| 17 | N-(2-Isopropyl-1,3- | DMSO-d6 11.65 (s, 1H), | 447 | 2-Methyl-5-{[3- |
| | thiazol-5-yl)-2-methyl-5- | 10.60 (s, 1H), 8.30 (m, | | (trifluoromethyl)benzoyl]- |
| | {[3- | 2H), 8.00-7.80 (m, 4H), | | amino}benzoic |
| | (trifluoromethyl)benzoyl]- | 7.42 (s, 1H), 7.35 (d, | | acid |
| | amino}benzamide | 1H), 3.20 (m, 1H), | | (Method 11) |
| | | 2.35 (s, 3H), 1.30 (d, 6H). | | |
| 18 | 2-Chloro-5-[(3- | DMSO-d6 12.00 (s, 1H), | 434 | 2-Chloro-5-[(3- |
| | chlorobenzoyl)amino-N- | 11.70 (s, 1H), 8.05 (m, | | chlorobenzoyl)amino]- |
| | (2-isopropyl-1,3-thiazol- | 2H), 7.95 (m, 2H), | | benzoic acid |
| | 5-yl)benzamide | 7.70 (d, 1H), 7.60 (m, 2H), | | (Method 9) |
| | | 7.50 (s, 1H), 3.28 (m, | | |
| | | 1H), 1.35 (d, 6H). | | |
| 19 | 5-{[3-(1-Cyano-1- | DMSO-d6 11.70 (s, 1H), | 446 | 5-{[3-(1-Cyano-1- |
| | methylethyl)benzoyl]amino}- | 10.46 (s, 1H), 8.05 (s, | | methylethyl)benzoyl]- |
| | N-(2-isopropyl-1,3- | 1H), 7.95 (m, 2H), | | amino}-2- |
| | thiazol-5-yl)-2- | 7.85 (d, 1H), 7.75 (d, 1H), | | methylbenzoic acid |
| | methylbenzamide | 7.60 (t, 1H), 7.46 (s, 1H), | | (Method 7) |
| | | 7.30 (d, 1H), 3.23 (m, | | |
| | | 1H), 2.35 (s, 3H), 1.75 (s, | | |
| | | 6H), 1.31 (d, 6H). | | |
| 20 | 2-Chloro-5-[(3,5- | DMSO-d6 12.10 (s, 1H), | 428 | 2-Chloro-5-[(3,5- |
| | dimethylbenzoyl)amino]- | 10.50 (s, 1H), 8.10 (s, | | dimethylbenzoyl)amino]- |
| | N-(2-isopropyl-1,3- | 1H), 7.98 (d, 1H), | | benzoic acid |
| | thiazol-5-yl)benzamide | 7.68 (m, 4H), 7.25 (s, 1H), | | (Method 10) |
| | | 3.30 (m, 1H), 2.37 (s, | | |
| | | 6H), 1.35 (d, 6H). | | |
| 21 | 2-Chloro-5-{[3-fluoro-5- | DMSO-d6 11.90 (s, 1H), | 486 | 2-Chloro-5-{[3- |
| | (trifluoromethyl)benzoyl]- | 10.80 (s, 1H), 8.20 (s, | | fluoro-5- |
| | amino}-N-(2-isopropyl- | 1H), 8.15 (d, 1H), | | (trifluoromethyl)benzoyl]- |
| | 1,3-thiazol-5- | 8.00 (m, 2H), 7.95 (d, 1H), | | amino}benzoic |
| | yl)benzamide | 7.60 (d, 1H), 7.46 (s, | | acid |
| | | 1H), 3.25 (m, 1H), | | (Method 12) |
| | | 1.30 (d, 6H). | | |
| 22 | 2-Chloro-N-(2-cyclopyl- | DMSO-d6 11.89 (s, 1H), | 466 | 2-Chloro-5-{[3- |
| | 1,3-thiazol-5-yl)-5-{[3- | 10.79 (s, 1H), 8.30 (m, | | (trifluoromethyl)benzoyl]- |
| | (trifluoromethyl)benzoyl]- | 2H), 8.00 (m, 3H), | | amino}benzoic |
| | amino}benzamide | 7.80 (m, 1H), 7.60 (d, 1H), | | acid |
| | | 7.40 (s, 1H), 2.35 (m, | | (Method 8) |
| | | 1H), 1.10 (m, 2H), | | |
| | | 0.98 (m, 2H). | | |
| 23 | 2-Chloro-N-(2- | DMSO-d6 12.15 (s, 1H), | 426 | 2-Chloro-5-[(3,5- |
| | cyclopropyl-1,3-thiazol-5- | 10.50 (s, 1H), 8.05 (s, | | dimethylbenzoyl)amino]- |
| | yl)-5-[(3,5- | 1H), 8.00 (d, 1H), | | benzoic acid |
| | dimethylbenzoyl)amino]benzamide | 7.60 (m, 4H), 7.20 (s, 1H), | | (Method 10) |
| | | 2.35 (m, 7H), 1.20 (m, | | |
| | | 2H), 1.05 (m, 2H). | | |
| 24 | 2-Chloro-N-(2- | DMSO-d6 11.90 (s, 1H), | 484 | 2-Chloro-5-{[3- |
| | cyclopropyl-1,3-thiazol-5- | 10.81 (s, 1H), 8.17 (m, | | fluoro-5- |
| | yl)-5-{[3-fluoro-5- | 2H), 8.00 (m, 3H), | | (trifluoromethyl)benzoyl]- |
| | (trifluoromethyl)benzoyl]- | 7.62 (d, 1H), 7.40 (s, 1H), | | amino}benzoic |
| | amino}benzamide | 2.31 (m, 1H), 1.08 (m, | | acid |
| | | 2H), 0.95 (m, 2H). | | (Method 12) |

### Example 25

### 5-{[3-Fluoro-5-(trifluoromethyl)benzoyl]amino}-2-methyl-N-(2-methyl-1,3-thiazol-5-yl)benzamide

To a solution of 5-amino-2-methyl-N-(2-methyl-1,3-thiazol-5-yl)benzamide (Method 25, 100 mg, 0.40 mmol) and 3-fluoro-5-(trifluoromethyl)benzoic acid (85 mg, 0.40 mmol) in anhydrous DMF (5 ml) was added HATU (154 mg, 0.40 mmol) and pyridine (5 eq.). After stirring for 16 hours, the reaction mixture was diluted with EtOAc, washed with water, dried (Na₂SO₄) and concentrated. Purification by column chromatography (Hex:EtOAc) gave 121 mg (68%) of a white solid.

¹H NMR Acetone-d6 10.70 (s, 1H) 9.94 (s, 1H) 8.19 (s, 1H) 8.08 (s, 1H) 8.04 (d, 1H) 7.80 (dd, 2H) 7.49 (s, 1H) 7.32 (d, 1H) 2.60 (s, 3H) 2.43 (s, 3H); m/z: 438.

### Examples 26-66

The following compounds were prepared by a procedure analogous to that described in Example 25 using 5-amino-2-methyl-N-(2-methyl-1,3-thiazol-5-yl)benzamide (Method 25) or 5-amino-2-chloro-N-(2-methyl-1,3-thiazol-5-yl)benzamide (Method 26) and the appropriate SM.

| | | | | Starting |
|---|---|---|---|---|
| Ex. | Compound | NMR | m/z | Material |
| 26 | 5-[(3-Chloro-5- | Acetone-d6 10.69 (s, 1H) | 404 | 3-Chloro-5- |
| | fluorobenzoyl)amino]-2- | 9.78 (s, 1H) | | fluorobenzoic |
| | methyl-N-(2-methyl-1,3- | 8.03 (d, 1H) 7.87 (s, 1H) | | acid |
| | thiazol-5-yl)benzamide | 7.72-7.80 (m, 2H) | | |
| | | 7.50 (m, 1H) 7.47 (s, 1H) | | |
| | | 7.30 (d, 1H) | | |
| | | 2.58 (s, 3H) 2.41 (s, 3H) | | |
| 27 | 5-[(3-Cyclopropyl-5- | Acetone-d6 10.68 (s, 1H) | 410 | 3-Cyclopropyl-5- |
| | fluorobenzoyl)amino]-2- | 9.66 (s, 1H) 8.03 (s, | | fluorobenzoic |
| | methyl-N-(2-methyl-1,3- | 1H) 7.77 (d, 1H) | | acid |
| | thiazol-5-yl)benzamide | 7.56 (s, 1H) 7.46-7.51 (m, 2H) | | (Method 31) |
| | | 7.28 (d, 1H) | | |
| | | 7.06 (m, 1H) 2.58 (s, 3H) | | |
| | | 2.41 (s, 3H) 2.06 (m, 1H) | | |
| | | 1.04 (m, 2H) | | |
| | | 0.80 (m, 2H) | | |
| 28 | 5-[(3-Chlorobenzoyl)amino]- | Acetone-d6 10.68 (s, 1H) | 386 | 3-Chlorobenzoic |
| | 2-methyl-N-(2-methyl-1,3- | 9.73 (s, 1H) | | acid |
| | thiazol-5-yl)benzamide | 7.94-8.05 (m, 3H) 7.79 (dd, | | |
| | | 1H) 7.62 (m, 1H) | | |
| | | 7.56 (m, 1H) 7.48 (s, 1H) | | |
| | | 7.38 (d, 1H) 2.58 (s, 3H) | | |
| | | 2.41 (s, 3H) | | |
| 29 | 5-[(3,4- | Acetone-d6 10.63 (s, 1H) | 420 | 3,4- |
| | Dichlorobenzoyl)amino]-2- | 9.77 (s, 1H) | | Dichlorobenzoic |
| | methyl-N-(2-methyl-1,3- | 8.18 (d, 1H) 8.04 (s, 1H) | | acid |
| | thiazol-5-yl)benzamide | 7.98 (dd, 1H) 7.79 (d, | | |
| | | 1H) 7.74 (d, 1H) | | |
| | | 7.47 (s, 1H) 7.30 (d, 1H) | | |
| | | 2.58 (s, 3H) 2.41 (s, 3H) | | |
| 30 | 5-[(3- | Acetone-d6 10.62 (s, 1H) | 392 | 3- |
| | Cyclopropylbenzoyl)amino]- | 9.58 (s, 1H) | | Cyclopropylbenzoic |
| | 2-methyl-N-(2-methyl-1,3- | 8.06 (d, 1H) 7.73-7.80 (m, | | acid |
| | thiazol-5-yl)benzamide | 2H) 7.69 (s, 1H) | | (Method 32) |
| | | 7.48 (s, 1H) 7.38 (t, 1H) | | |
| | | 7.27-7.31 (m, 2H) | | |
| | | 2.58 (s, 3H) 2.41 (s, 3H) | | |
| | | 2.06 (m, 1H) | | |
| | | 1.01 (m, 2H) 0.74 (m, 2H) | | |
| 31 | 5-[(3,5- | Acetone-d6 10.61 (s, 1H) | 380 | 3,5- |
| | Dimethylbenzoyl)amino]-2- | 9.52 (s, 1H) | | Dimethylbenzoic |
| | methyl-N-(2-methyl-1,3- | 8.05 (d, 1H) 7.80 (dd, 1H) | | acid |
| | thiazol-5-yl)benzamide | 7.60 (s, 2H) 7.48 (s, 1H) | | |
| | | 7.28 (d, 1H) | | |
| | | 7.21 (s, 1H) 2.59 (s, 3H) | | |
| | | 2.41 (s, 3H) 2.35 (s, 6H) | | |
| 32 | 2-Methyl-5-[(3- | CD₃OD 7.90 (d, 1H) | 366 | 3-Methylbenzoic |
| | methylbenzoyl)amino]-N-(2- | 7.65-7.75 (m, 3H) | | acid |
| | methyl-1,3-thiazol-5- | 7.38-7.42 (m, 3H) | | |
| | yl)benzamide | 7.30 (d, 1H) 2.63 (s, 3H) | | |
| | | 2.41 (s, 6H) | | |
| 33 | 2,6-Dichloro-N-(4-methyl-3- | Acetone-d6 10.72 (s, 1H) | 421 | 2,6- |
| | {[(2-methyl-1,3-thiazol-5- | 9.97 (s, 1H) 8.00 (s, | | Dichloroisonicotinic |
| | yl)amino]carbonyl}phenyl)isonicotinamide | 1H) 7.94 (s, 2H) | | acid |
| | | 7.76 (d, 1H) 7.47 (s, 1H) | | |
| | | 7.29 (d, 1H) 2.58 (s, 3H) | | |
| | | 2.40 (s, 3H) | | |
| 34 | 2-Methyl-5-{[(3- | CD₃OD 7.75 (d, 1H) | 372 | 3- |
| | methylcyclohexyl)carbonyl]amino}- | 7.45 (dd, 1H) 7.36 (s, | | Methylcyclohexanecarboxylic |
| | N-(2-methyl-1,3- | 1H) 7.19 (d, 1H) | | acid |
| | thiazol-5-yl)benzamide | 0.86-1.81 (m, 13H) | | |
| 35 | 2-Methyl-N-(2-methyl-1,3- | DMSO-d6 11.56 (s, 1H) | 332 | Pentanoic acid |
| | thiazol-5-yl)-5- | 9.98 (s, 1H) | | |
| | (pentanoylamino)benzamide | 7.74 (d, 1H) 7.68 (dd, 1H) | | |
| | | 7.39 (s, 1H) 7.23 (d, 1H) | | |
| | | 2.55 (s, 3H) | | |
| | | 2.28-2.32 (m, 5H) | | |
| | | 1.51-1.61 (m, 2H) | | |
| | | 1.27-1.34 (m, 2H) 0.88 (t, 3H) | | |
| 36 | 2-Methyl-5-[(4- | Acetone-d6 10.67 (s, 1H) | 360 | 4-Methylhexanoic |
| | methylhexanoyl)amino]-N- | 9.24 (s, 1H) 7.91 (s, | | acid |
| | (2-methyl-1,3-thiazol-5- | 1H) 7.59 (dd, 1H) | | |
| | yl)benzamide | 7.49 (s, 1H) 7.21 (d, 1H) | | |
| | | 2.60 (s, 3H) | | |
| | | 2.33-2.41 (m, 5H) | | |
| | | 1.67-1.77 (m, 1H) | | |
| | | 1.33-1.44 (m, 3H) | | |
| | | 1.13-1.23 (m, 1H) | | |
| | | 0.87-0.91 (m, 6H) | | |
| 37 | 2-Chloro-5- | DMSO-d6 12.00 (s, | 427 | Cyclohexyl- |
| | {[cyclohexyl(difluoro)acetyl]- | 1H), 10.80 9s, 1H), | | (difluoro) acetic |
| | amino}-N-(2-methyl-1,3- | 8.00 (s, 1H), 7.89 (d, | | acid |
| | thiazol-5-yl)benzamide | 1H), 7.60 (d, 1H), | | (Method 13) |
| | | 7.50 (s, 1H), 2.68 (s, 3H), | | |
| | | 2.25 (m, 1H), | | |
| | | 1.80-1.60 (m, 5H), 1.20 (m, 5H) | | |
| 38 | 2-Methyl-5-[(4- | MeOD 7.76 (d, 1H) | 346 | 4- |
| | methylpentanoyl)amino]-N- | 7.65 (dd, 1H) 7.35 (d, | | Methylpentanoic |
| | (2-methyl-1,3-thiazol-5- | 1H) 7.31 (s, 1H) | | acid |
| | yl)benzamide | 2.71 (s, 3H) 2.54 (s, 3H) | | |
| | | 2.26-2.31 (m, 2H) | | |
| | | 1.45-1.51 (m, 3H) 0.85 (d, | | |
| | | 6H) | | |
| 39 | 5- | MeOD 7.79 (s, 1H) | 344 | Cyclopentanecarboxylic |
| | [(Cyclopentylcarbonyl)amino]- | 7.49 (m, 1H) 7.40 (s, | | acid |
| | 2-methyl-N-(2-methyl- | 1H) 7.24 (d, 1H) | | |
| | 1,3-thiazol-5-yl)benzamide | 2.78-2.83 (m, 1H) 2.63 (s, | | |
| | | 3H) 2.38 (s, 3H) | | |
| | | 1.62-1.95 (m, 8H) | | |
| 40 | 5-[(sec- | MeOD 7.70 (d, 1H) | 362 | sec-Butoxyacetic |
| | Butoxyacetyl)amino]-2- | 7.47 (dd, 1H) 7.30 (s, | | acid |
| | methyl-N-(2-methyl-1,3- | 1H) 7.18 (d, 1H) | | |
| | thiazol-5-yl)benzamide | 3.97 (s, 2H) 3.24-3.27 (m, | | |
| | | 2H) 2.53 (s, 3H) | | |
| | | 2.29 (s, 3H) 1.82-1.87 (m, | | |
| | | 1H) 0.84-0.87 (m, 6H) | | |
| 41 | 2-Chloro-5-[(3,4- | DMSO-D6 11.87 (s, | 442 | 3,4- |
| | dichlorobenzoyl)amino]-N- | 1H) 10.67 (s, 1H) | | Dichlorobenzoic |
| | (2-methyl-1,3-thiazol-5- | 8.24 (d, 1H) 8.02 (d, 1H) | | acid |
| | yl)benzamide | 7.92-7.97 (m, 2H) | | |
| | | 7.85 (d, 1H) 7.60 (d, 1H) | | |
| | | 7.42 (s, 1H) 2.58 (s, | | |
| | | 3H) | | |
| 42 | 2-Chloro-5-[(3-chloro-5- | Acetone D-6 10.85 (s, | 424 | 3-Chloro-5- |
| | fluorobenzoyl)amino]-N-(2- | 1H) 9.95 (s, 1H) | | fluorobenzoic |
| | methyl-1,3-thiazol-5- | 8.13 (d, 1H) 7.95 (dd, 1H) | | acid |
| | yl)benzamide | 7.88 (s, 1H) 7.75 (d, | | |
| | | 1H) 7.50-7.54 (m, 3H) | | |
| | | 2.61 (s, 3H) | | |
| 43 | 2-Chloro-5-[(3,4- | Acetone-D6 10.80 (s, | 400 | 3,4- |
| | dimethylbenzoyl)amino]-N- | 1H) 9.71 (s, 1H) | | Dimethylbenzoic |
| | (2-methyl-1,3-thiazol-5- | 8.14 (d, 1H) 7.95 (dd, 1H) | | acid |
| | yl)benzamide | 7.77 (s, 1H) 7.72 (dd, | | |
| | | 1H) 7.45-7.49 (m, 2H) | | |
| | | 7.25 d, 1H) 2.60 (s, 3H) | | |
| | | 2.30 (s, 6H) | | |
| 44 | N-(4-Chloro-3-{[(2-methyl- | DMSO-D6 11.90 (s, | 387 | 6- |
| | 1,3-thiazol-5- | 1H) 10.81 (s, 1H) | | Methylpyridine- |
| | yl)amino]carbonyl}phenyl)- | 8.25 (d, 1H) 8.15 (dd, 1H) | | 2-carboxylic acid |
| | 6-methylpyridine-2- | 8.00 (d, 2H) 7.64 (d, | | |
| | carboxamide | 1H) 7.60 (t, 1H) | | |
| | | 7.46 (s, 1H) 2.68 (s, 3H) | | |
| | | 2.63 (s, 3H) | | |
| 45 | 3-Chloro-N-(4-chloro-3-{[(2- | MeOD 8.56 (d, 1H) | 408 | 3- |
| | methyl-1,3-thiazol-5- | 8.04 (d, 1H) 7.95 (s, | | Chloroisonicotinic |
| | yl)amino]carbonyl}phenyl)isonicotinamide | 1H) 7.82-7.88 (m, 2H) | | acid |
| | | 7.53 (d, 1H) 7.44 (s, | | |
| | | 1H) 2.66 (s, 3H) | | |
| 46 | 2-Chloro-5-{[(3- | MeOD 7.76 (d, 1H) | 393 | 3- |
| | methylcyclohexyl)carbonyl]amino}- | 7.55 (dd, 1H) 7.33 (d, | | Methylcyclohexanecarboxylic |
| | N-(2-methyl-1,3- | 1H) 7.30 (s, 1H) | | acid |
| | thiazol-5-yl)benzamide | 3.24 9 (S, 2H) 2.53 (s, 3H) | | |
| | | 2.24-2.32 (m, 1H) | | |
| | | 1.23-1.75 (m, 6H) | | |
| | | 0.99-1.11 (m, 1H) 0.84 (d, 3H) | | |
| 47 | 2-Chloro-5- | MeOD 7.90 (s, 1H) | 392 | Cyclohexylacetic |
| | [(cyclohexylacetyl)amino]- | 7.68 (dd, 1H) 7.46 (d, | | acid |
| | N-(2-methyl-1,3-thiazol-5- | 1H) 7.43 (s, 1H) | | |
| | yl)benzamide | 2.66 (s, 3H) 2.26 (d, 2H) | | |
| | | 1.68-1.79 (m, 6H) | | |
| | | 1.20-1.35 (m, 3H) | | |
| | | 1.03-1.11 (m, 2H) | | |
| 48 | 2-Chloro-5-[(3- | MeOD 11.90 (s, 1H) | 386 | 3-Methylbenzoic |
| | methylbenzoyl)amino]-N-(2- | 10.81 (s, 1H) 8.25 (d, | | acid |
| | methyl-1,3-thiazol-5- | 1H) 8.15 (dd, 1H) | | |
| | yl)benzamide | 8.00 (d, 1H) 7.64 (d, 1H) | | |
| | | 7.60 (t, 1H) 7.46 (s, | | |
| | | 1H) 2.58 (s, 3H) | | |
| | | 2.63 (s, 3H) | | |
| 49 | N-(4-Chloro-3-{[(2-methyl- | MeOD 8.76 (s, 1H) | 387 | 5-Methylnicotinic |
| | 1,3-thiazol-5- | 8.46 (s, 1H) 8.07 (s, | | acid |
| | yl)amino]carbonyl}phenyl)- | 1H) 7.91 (d, 1H) | | |
| | 5-methylnicotinamide | 7.73 (dd, 1H) 7.40 (d, 1H) | | |
| | | 7.31 (s, 1H) 2.53 (s, | | |
| | | 3H) 2.34 (s, 3H) | | |
| 50 | 2-Chloro-N-(2-methyl-1,3- | MeOD 7.77 (d, 1H) | 352 | Pentanoic acid |
| | thiazol-5-yl)-5- | 7.56 (dd, 1H) 7.35 (d, | | |
| | (pentanoylamino)benzamide | 1H) 7.31 (s, 1H) | | |
| | | 2.54 (s, 3H) 2.28 (t, 2H) | | |
| | | 1.52-1.62 (m, 2H) | | |
| | | 1.24-1.36 (m, 2H) 0.86 (t, | | |
| | | 3H) | | |
| 51 | 2-Chloro-5-[(4- | MeOD 7.77 (d, 1H) | 366 | 4- |
| | methylpentanoyl)amino]-N- | 7.56 (dd, 1H) 7.35 (d, | | Methylpentanoic |
| | (2-methyl-1,3-thiazol-5- | 1H) 7.32 (s, 1H) | | acid |
| | yl)benzamide | 2.71 (s, 3H) 2.30 (t, 2H) | | |
| | | 1.48-1.53 (m, 2H) | | |
| | | 0.84-0.86 (m, 7H) | | |
| 52 | 2-Chloro-5-[(3- | MeOD 7.78 (s, 1H) | 366 | 3- |
| | methylpentanoyl)amino]-N- | 7.67 (dd, 1H) 7.35 (d, | | Methylpentanoic |
| | (2-methyl-1,3-thiazol-5- | 1H) 7.32 (s, 1H) | | acid |
| | yl)benzamide | 2.71 (s, 3H) 2.29 (dd, 1H) | | |
| | | 2.06 (dd, 1H) | | |
| | | 1.80-1.88 (m, 1H) 1.30-1.38 (m, | | |
| | | 1H) 1.13-1.23 (m, 1H) | | |
| | | 0.82-0.89 (m, 6H) | | |
| 53 | 2-Methyl-5-{[(4- | MeOD 7.78 (d, 1H) | 372 | 4- |
| | methylcyclohexyl)carbonyl]amino}- | 7.48 (d, 1H) 7.40 (d, | | Methylcyclohexanecarboxylic |
| | N-(2-methyl-1,3- | 1H) 7.23 (d, 1H) | | acid |
| | thiazol-5-yl)benzamide | 2.63 (s, 3H) 2.37 (s, 3H) | | |
| | | 1.77-1.90 m, 4H) | | |
| | | 1.49-1.64 (m, 5H) 0.99 (d, | | |
| | | 2H) 0.91 (d, 2H) | | |
| 54 | 2-Methyl-5-{[(4- | MeOD 7.79 (d, 1H) | 386 | (4- |
| | methylcyclohexyl)acetyl]amino}- | 7.49 (dd, 1H) 7.40 (s, | | Methylcyclohexyl)acetic |
| | N-(2-methyl-1,3-thiazol- | 1H) 7.24 (d, 1H) | | acid |
| | 5-yl)benzamide | 2.63 (s, 3H) 2.37 (s, 3H) | | |
| | | 2.22 (d, 1H) 2.07 (m, | | |
| | | 1H) 1.66-1.78 (m, | | |
| | | 2H) 1.42-1.56 (m, 3H) | | |
| | | 1.28-1.36 (m, 2H) | | |
| | | 0.97-1.08 (m, 1H) 0.94 (d, | | |
| | | 2H) 0.88 (d, 2H) | | |
| 55 | 2-Methyl-5-[(2- | MeOD 7.70 (d, 1H) | 332 | 2-Methylbutanoic |
| | methylbutanoyl)amino]-N- | 7.41 (dd 1H) 7.30 (s, | | acid |
| | (2-methyl-1,3-thiazol-5- | 1H) 7.15 (d, 1H) | | |
| | yl)benzamide | 2.53 (s, 3H) 2.28-2.36 (m, | | |
| | | 1H) 2.28 (s, 3H) | | |
| | | 1.52-1.67 (m, 1H) | | |
| | | 1.33-1.44 (m, 1H) 1.07 (d, 3H) | | |
| | | 0.84 (t, 3H) | | |
| 56 | 2-Methyl-5-[(2- | MeOD 7.70 (d, 1H) | 360 | 2-Methylhexanoic |
| | methylhexanoyl)amino]-N- | 7.41 (dd, 1H) 7.30 (s, | | acid |
| | (2-methyl-1,3-thiazol-5- | 1H) 7.15 (d, 1H) | | |
| | yl)benzamide | 2.53 (s, 3H) 2.31-2.41 (m, | | |
| | | 1H) 2.28 (s, 3H) | | |
| | | 1.53-1.65 (m, 1H) | | |
| | | 1.18-1.38 (m, 5H) 1.07 (d, 3H) | | |
| | | 0.80 (t, 3H) | | |
| 57 | 5-[(Cyclopropylacetyl)amino]- | MeOD 7.69 (d, 1H) | 330 | Cyclopropylacetic |
| | 2-methyl-N-(2-methyl-1,3- | 7.40 (dd, 1H) 7.30 (s, | | acid |
| | thiazol-5-yl)benzamide | 1H) 7.15 (d, 1H) | | |
| | | 2.53 (s, 3H) 2.28 (s, 3H) | | |
| | | 2.16 (d, 2H) | | |
| | | 0.98-1.05 (m, 1H) 0.43-0.49 (m, | | |
| | | 2H) 0.11-0.16 (m, 2H) | | |
| 58 | 2-Methyl-N-(2-methyl-1,3- | MeOD 7.67 (s, 1H) | 426 | 3- |
| | thiazol-5-yl)-5-({[3- | 7.35 (d, 1H) 7.30 (s, | | (Trifluoromethyl)- |
| | (trifluoromethyl)cyclohexyl]- | 1H) 7.12 (d, 1H) | | cyclohexanecarboxylic |
| | carbonyl}amino)benzamide | 2.51 (s, 3H) 2.26 (s, 3H) | | acid |
| | | 2.06-2.12 (m, 1H) | | |
| | | 1.90-1.98 (m, 2H) | | |
| | | 1.59-1.81 (m, 7H) | | |
| 59 | 5- | MeOD 7.67 (d, 1H) | 358 | Cyclopentylacetic |
| | [(Cyclopentylacetyl)amino]- | 7.37 (dd, 1H) 7.28 (s, | | acid |
| | 2-methyl-N-(2-methyl-1,3- | 1H) 7.12 (d, 1H) | | |
| | thiazol-5-yl)benzamide | 2.50 (s, 3H) 2.25 (s, 3H) | | |
| | | 2.23 (s, 2H) | | |
| | | 2.12-2.19 (m, 1H) 1.69-1.76 (m, | | |
| | | 2H) 1.43-1.58 (m, 4H) | | |
| | | 1.06-1.17 (m, 2H) | | |
| 60 | 5-{[(4,4- | MeOD 7.68 (s, 1H) | 394 | 4,4- |
| | Difluorocyclohexyl)carbonyl]- | 7.37 (d, 1H) 7.30 (s, | | Difluorocyclohexanecarboxylic |
| | amino}-2-methyl-N-(2- | 1H) 7.14 (d, 1H) | | acid |
| | methyl-1,3-thiazol-5- | 2.52 (s, 3H) 2.26 (s, 3H) | | |
| | yl)benzamide | 2.32-2.40 (1H) | | |
| | | 1.97-2.07 (m, 2H) | | |
| | | 1.67-1.86 (m, 6H) | | |
| 61 | N-(4-Chloro-3-{[(2-methyl- | MeOD 7.92 (d, 1H) | 390 | 1,5-Dimethyl-1H- |
| | 1,3-thiazol-5- | 7.74 (dd, 1H) 7.39 (d, | | pyrazole-3- |
| | yl)amino]carbonyl}phenyl)- | 1H) 7.32 (s, 1H) | | carboxylic acid |
| | 1,5-dimethyl-1H-pyrazole-3- | 6.49 (s, 1H) 3.77 (s, 3H) | | |
| | carboxamide | 2.54 (s, 3H) 2.24 (s, | | |
| | | 3H) | | |
| 62 | N-(4-Chloro-3-{[(2-methyl- | MeOD 8.02 (d, 1H) | 376 | 5-Methyl-1H- |
| | 1,3-thiazol-5- | 7.83 (d, 1H) 7.47 (d, | | pyrazole-3- |
| | yl)amino]carbonyl}phenyl)- | 1H) 7.42 (s, 1H) | | carboxylic acid |
| | 5-methyl-1H-pyrazole-3- | 6.57 (s, 1H) 2.64 (s, 3H) | | |
| | carboxamide | 2.33 (s, 3H) | | |
| 63 | 1,5-Dimethyl-N-(4-methyl-3- | MeOD 7.80 (d, 1H) | 370 | 1,5-Dimethyl-1H- |
| | {[(2-methyl-1,3-thiazol-5- | 7.67 (dd, 1H) 7.32 (s, | | pyrazole-3- |
| | yl)amino]carbonyl}phenyl)- | 1H) 7.19 (d, 1H) | | carboxylic acid |
| | 1H-pyrazole-3-carboxamide | 6.49 (s, 1H) 3.77 (s, 3H) | | |
| | | 2.54 (s, 3H) 2.31 (s, | | |
| | | 3H) 2.24 (s, 3H) | | |
| 64 | 5-Methyl-N-(4-methyl-3- | MeOD 7.91 (d, 1H) | 356 | 5-Methyl-1H- |
| | {[(2-methyl-1,3-thiazol-5- | 7067 (dd, 1H) 7.42 (s, | | pyrazole-3- |
| | yl)amino]carbonyl}phenyl)- | 1H) 7.30 (s, 1H) | | carboxylic acid |
| | 1H-pyrazole-3-carboxamide | 6.59 (s, s, 1H) 2.64 (s, 3H) | | |
| | | 2.40 (s, 3H) 2.34 (s, | | |
| | | 3H) | | |
| 65 | 1-tert-Butyl-5-methyl-N-(4- | DMSO-D6 11.62 (s, | 411 | 1-tert-Butyl-5- |
| | methyl-3-{[(2-methyl-1,3- | 1H) 9.73 (s, 1H) | | methyl-1H- |
| | thiazol-5- | 7.88-7.95 (m, 2H) 7.41 (s, | | pyrazole-3- |
| | yl)amino]carbonyl}phenyl)- | 1H) 7.28 (d, 1H) | | carboxylic acid |
| | 1H-pyrazole-3-carboxamide | 6.58 (s, 1H) 2.57 (s, 3H) | | |
| | | 2.48 (s, 3H) 2.33 (s, | | |
| | | 3H) 1.64 (s, 9H) | | |
| 66 | 1-Isopropyl-N-(4-methyl-3- | Acetone-D6 10.62 (s, | 384 | 1-Isopropyl-1H- |
| | {[(2-methyl-1,3-thiazol-5- | 1H) 9.40 (s, 1H) | | pyrazole-3- |
| | yl)amino]carbonyl}phenyl)- | 8.04 (d, 1H) 7.80-7.85 (m, | | carboxylic acid |
| | 1H-pyrazole-3-carboxamide | 2H) 7.48 (s, 1H) | | |
| | | 7.27 (d, 1H) 6.74 (d, 1H) | | |
| | | 4.58-4.67 (m, 1H) | | |
| | | 2.58 (s, 3H) 2.40 (s, 3H) | | |
| | | 1.52 (d, 6H) | | |

### Example 67

### 2-Chloro-N-{2-[(dimethylamino)methyl]-1,3-thiazol-5-yl}-5-{[3-(trifluoromethyl)benzoyl]amino}benzamide

To a 10 mL round bottom flask charged with a magnetic stir bar and 2-chloro-N-(2-formyl-1,3-thiazol-5-yl)-5-{[3 (trifluoromethyl)benzoyl]amino}benzamide (0.123 g, 0.272 mmol) (Example 86) was added anhydrous THF (3 mL). A 2M solution of dimethylamine in THF (0.34 mL, 0.68 mmol) was added to the reaction followed by the addition of glacial acetic acid (0.050 mL, 0.83 mmol). With stirring, NaBH(OAc)₃ (0.23 g, 1.09 mmol) was added and the reaction was warmed to 50° C. and allowed to stir at this temperature for 5 h before being diluted with a saturated aqueous solution of NaHCO₃ (˜5 mL). The mixture was then poured into a separatory funnel and extracted with EtOAc (˜50 mL) and washed with 2×50 mL of saturated aqueous solution of NaHCO₃. The organic phase was separated, dried with MgSO₄, filtered, and conc. in vacuo to yield the crude product. The crude product was purified via reverse phase HPLC using MeCN/H₂O (1:1) as eluent which afforded the title compound as an off white solid; DMSO-D6 12.32 (s, 1H) 10.79 (s, 1H) 8.24-8.34 (m, 2H) 8.12 (d, 1H) 7.89-8.03 (m, 2H) 7.81 (t, 1H) 7.72 (s, 1H) 7.56-7.67 (m, 1H) 4.56-4.69 (m, 2H) 2.82 (s, 6H); m/z: 483.

### Examples 68-81

The following compounds were prepared by a procedure analogous to that described in Example 67 using 2-chloro-N-(2-formyl-1,3-thiazol-5-yl)-5-{[3(trifluoromethyl)benzoyl]amino}benzamide (Example 86), 2-chloro-5-[(3,5-dimethylbenzoyl)amino]-N-(2-formyl-1,3-thiazol-5-yl)benzamide (Example 87), or 5-{[3-(1-cyano-1-methylethyl)benzoyl]amino}-N-(2-formyl-1,3-thiazol-5-yl)-2-methylbenzamide (Example 88) and the appropriate SM.

| Ex. | Compound | NMR | m/z | Starting Material |
|---|---|---|---|---|
| 68 | 2-Chloro-N-{2-[(4- | DMSO-D6 11.98 (s, 1H) | 538 | 1-Methypiperazine |
| | methylpiperazin-1- | 10.76 (s, 1H) | | |
| | yl)methyl]-1,3-thiazol-5- | 9.50 (s, 1H) 8.23-8.34 (m, | | |
| | yl}-5-{[3- | 2H) 8.07 (d, 1H) | | |
| | (trifluoromethyl)benzoyl]amino}- | 7.96-8.03 (m, 1H) | | |
| | benzamide | 7.91 (dd, 1H) 7.81 (t, 1H) | | |
| | | 7.61 (d, 1H) 7.48 (s, 1H) | | |
| | | 3.89 (s, 3H) 3.38 (s, | | |
| | | 2H) 3.07 (s, 4H) | | |
| | | 2.80 (s, 3H) | | |
| 69 | 2-Chloro-N-[2-(piperidin- | DMSO-D6 12.32 (s, 1H) | 523 | Piperidine |
| | 1-ylmethyl)-1,3-thiazol-5- | 10.79 (s, 1H) | | |
| | yl]-5-{[3- | 8.30 (s, 1H) 8.27 (d, 1H) | | |
| | (trifluoromethyl)benzoyl]amino}- | 8.12 (d, 1H) 8.00 (d, 1H) | | |
| | benzamide | 7.92 (dd, 1H) | | |
| | | 7.81 (t, 1H) 7.71 (s, 1H) | | |
| | | 7.57-7.66 (m, 1H) | | |
| | | 4.63 (s, 2H) 3.44 (d, 2H) | | |
| | | 2.98 (s, 2H) | | |
| | | 1.70-1.86 (m, 3H) 1.66 (s, 3H) | | |
| 70 | 2-Chloro-N-{2- | DMSO-D6 12.24 (s, 1H) | 469 | Methylamine |
| | [(methylamino)methyl]- | 10.78 (s, 1H) | | |
| | 1,3-thiazol-5-yl}-5-{[3- | 9.10 (s, 1H) 8.24-8.35 (m, | | |
| | (trifluoromethyl)benzoyl]amino}- | 2H) 8.11 (d, 1H) | | |
| | benzamide | 7.89-8.04 (m, 2H) 7.81 (t, | | |
| | | 1H) 7.58-7.67 (m, 2H) | | |
| | | 4.48 (s, 2H) 2.63 (s, | | |
| | | 3H) | | |
| 71 | 2-Chloro-N-[2-(morpholin- | DMSO-D6 12.27 (s, 1H) | 525 | Morpholine |
| | 4-ylmethyl)-1,3-thiazol-5- | 10.78 (s, 1H) | | |
| | yl]-5-{[3- | 8.24-8.34 (m, 2H) 8.11 (d, 1H) | | |
| | (trifluoromethyl)benzoyl]amino}- | 7.89-8.03 (m, 2H) | | |
| | benzamide | 7.81 (t, 1H) | | |
| | | 7.57-7.71 (m, 2H) 4.57 (s, 2H) | | |
| | | 3.78 (m, 4H) 3.17 (m, | | |
| | | 4H) | | |
| 72 | 2-Chloro-N-[2-({[2- | DMSO-D6 12.23 (s, 1H) | 526 | N,N- |
| | (dimethylamino)ethyl]amino}- | 10.79 (s, 1H) | | Dimethylethane- |
| | methyl)-1,3-thiazol-5- | 8.23-8.34 (m, 2H) 8.13 (d, 1H) | | 1,2-diamine |
| | yl]-5-{[3- | 8.00 (d, 1H) | | |
| | (trifluoromethyl)benzoyl]amino}- | 7.91 (dd, 1H) 7.81 (t, 1H) | | |
| | benzamide | 7.56-7.66 (m, 2H) | | |
| | | 4.50 (s, 2H) 3.36 (s, 4H) | | |
| | | 2.84 (s, 6H) | | |
| 73 | N-[2-(Azetidin-1- | DMSO-D6 12.27 (s, 1H) | 495 | Azetidine |
| | ylmethyl)-1,3-thiazol-5- | 10.78 (s, 1H) | | |
| | yl]-2-chloro-5-{[3- | 8.30 (s, 1H) 8.27 (d, 1H) | | |
| | (trifluoromethyl)benzoyl]amino}- | 8.11 (d, 1H) | | |
| | benzamide | 7.89-8.03 (m, 2H) 7.81 (t, 1H) | | |
| | | 7.56-7.69 (m, 2H) | | |
| | | 4.72 (s, 2H) 4.12 (m, 4H) | | |
| | | 2.40 (m, 2H) | | |
| 74 | 2-Chloro-N-{2- | DMSO-D6 12.24 (s, 1H) | 509 | Cyclobutylamine |
| | [(cyclobutylamino)methyl]- | 10.77 (s, 1H) | | |
| | 1,3-thiazol-5-yl}-5-{[3- | 8.30 (s, 1H) 8.27 (d, 1H) | | |
| | (trifluoromethyl)benzoyl]amino}- | 8.11 (d, 1H) | | |
| | benzamide | 7.89-8.03 (m, 2H) 7.81 (t, 1H) | | |
| | | 7.57-7.70 (m, 2H) | | |
| | | 4.41 (s, 2H) 3.76 (s, 1H) | | |
| | | 2.06-2.20 (m, 4H) | | |
| | | 1.70-1.84 (m, 2H) | | |
| 75 | 2-Chloro-N-(2- | DMSO-D6 12.24 (s, 1H) | 509 | (Cyclopropylmethyl)- |
| | {[(cyclopropylmethyl)amino]- | 10.77 (s, 1H) | | amine |
| | methyl}-1,3-thiazol-5- | 8.23-8.33 (m, 2H) 8.12 (d, 1H) | | |
| | yl)-5-{[3- | 7.89-8.03 (m, 2H) | | |
| | (trifluoromethyl)benzoyl]amino}- | 7.81 (t, 1H) | | |
| | benzamide | 7.57-7.69 (m, 2H) 4.52 (s, 2H) | | |
| | | 2.91 (s, 2H) 1.05 (d, 1H) | | |
| | | 0.53-0.63 (m, 2H) | | |
| | | 0.30-0.40 (m, 2H) | | |
| 76 | 2-Chloro-N-(2-{[(2- | DMSO-D6 12.30 (s, 1H) | 527 | 2-Methoxy-N- |
| | methoxyethyl)(methyl)amino]- | 10.76 (s, 1H) | | methylethanamine |
| | methyl}-1,3-thiazol-5- | 8.28 (s, 2H) 8.12 (s, 1H) | | |
| | yl)-5-{[3- | 7.96 (s, 2H) 7.81 (s, 1H) | | |
| | (trifluoromethyl)benzoyl]amino}- | 7.66 (s, 2H) 4.64 (s, | | |
| | benzamide | 2H) 3.70 (s, 3H) | | |
| | | 3.31 (s, 4H) 2.78 (s, 3H) | | |
| 77 | 2-Chloro-N-(2-{[4- | DMSO-D6 12.30 (s, 1H) | 553 | Piperidin-4- |
| | (hydroxymethyl)piperidin- | 10.76 (s, 1H) | | ylmethanol |
| | 1-yl]methyl}-1,3-thiazol-5- | 8.28 (s, 2H) 8.12 (s, 1H) | | |
| | yl)-5-{[3- | 7.96 (s, 2H) 7.81 (s, 1H) | | |
| | (trifluoromethyl)benzoyl]amino}- | 7.66 (s, 2H) 4.64 (s, | | |
| | benzamide | 2H) 3.70 (s, 4H) | | |
| | | 3.31 (s, 4H) 2.78 (s, 3H) | | |
| 78 | 2-Chloro-N-{2- | DMSO-D6 12.18 (s, 1H) | 495 | Cyclopropylamine |
| | [(cyclopropylamino)methyl]- | 10.72 (s, 1H) | | |
| | 1,3-thiazol-5-yl}-5-{[3- | 8.18-8.28 (m, 2H) 8.06 (d, 1H) | | |
| | (trifluoromethyl)benzoyl]amino}- | 7.84-7.98 (m, 2H) | | |
| | benzamide | 7.75 (t, 1H) | | |
| | | 7.51-7.63 (m, 2H) 4.54 (s, 2H) | | |
| | | 2.73 (s, 1H) | | |
| | | 0.66-0.79 (m, 4H) | | |
| 79 | 2-Chloro-5-[(3,5- | DMSO-D6 12.31 (s, | 485 | Morpholine |
| | dimethylbenzoyl)amino]- | 1H), 10.50 (s, 1H), | | |
| | N-[2-(morpholin-4- | 8.15 (s, 1H), 7.95 (d, 1H), | | |
| | ylmethyl)-1,3-thiazol-5- | 7.70 (s, 1H), 7.59 (m, | | |
| | yl]benzamide | 3H), 7.25 9s, 1H), | | |
| | | 4.70 (s, 2H), 4.00-3.30 (m, | | |
| | | 8H), 2.36 (s, 6H). m/z | | |
| | | 485.5 | | |
| 80 | 2-Chloro-N-{2- | DMSO-D6 14.50 (s, | 455 | Cyclopropylamine |
| | [(cyclopropylamino)methyl]- | 1H), 11.90 (s, br, 2H), | | |
| | 1,3-thiazol-5-yl}-5-[(3,5- | 10.40 (s, 1H), 10.21 (d, | | |
| | dimethylbenzoly)amino]benzamide | 1H), 9.96 (s, 1H), | | |
| | | 9.89 (m, 3H), 9.50 (s, 1H), | | |
| | | 6.85 (s, 2H), 4.60 (s, | | |
| | | 6H), 3.15 (m, 2h), 3.00 | | |
| | | 9m, 2H) | | |
| 81 | 5-{[3-(1-Cyano-1- | DMSO-D6 12.00 (s, | 503 | 5-{[3-(1-Cyano-1- |
| | methylethyl)benzoyl]amino}- | 1H), 10.49 (s, 1H), | | methylethyl)benzoyl]- |
| | 2-methyl-N-(2- | 8.01 (s, 1H), 7.99 (s, 1H), | | amino}-N-(2- |
| | morpholin-4-ylmethyl)- | 7.89 (d, 1H), 7.76 (d, | | formyl-1,3-thiazol- |
| | 1,3-thiazol-5-yl)benzamide | 1H), 7.69 (m, 2H), | | 5-yl)-2- |
| | | 7.55 (t, 1H), 7.27 (d, 1H), | | methylbenzamide |
| | | 4.60 (s, 2H), | | (Example 88) |
| | | 3.86-3.70 (m, 4H), 3.30-3.10 (m, | | |
| | | 4H), 2.26 (s, 3H), | | |
| | | 1.65 (s, 6H) | | |

### Example 82

### 2-Chloro-N-(2-formyl-1,3-thiazol-5-yl)-5-{[3-(trifluoromethyl)benzoyl]amino}benzamide

To a 25 mL round bottom flask charged with a magnetic stir bar and 2-chloro-N-(2-formyl-1,3-thiazol-5-yl)-5-{[3(trifluoromethyl)benzoyl]amino}benzamide (0.28 g, 0.58 mmol) (Example 86) was added methanol (5 mL). Sodium borohydride (0.66 g, 1.74 mmol) was added and the reaction was allowed to stir at rt for 1 h before being diluted with a saturated aqueous solution of NH₄Cl (˜20 mL). The mixture was then poured into a separatory funnel and extracted with EtOAc (˜50 mL). The organic phase was separated, dried with MgSO₄, filtered, and conc. in vacuo to yield the crude product. The crude oil was purified on SiO₂ (40 g) using EtOAc as eluent which afforded the title compound as a white solid; DMSO-D6 11.88 (s, 1H) 10.74 (s, 1H) 8.24-8.32 (m, 2H) 7.93-8.04 (m, 3H) 7.80 (t, 1H) 7.60 (d, 1H) 7.49 (s, 1H) 4.66 (s, 2H); m/z: 456.

### Example 83

### 2-Chloro-N-[2-(1-hydroxyethyl)-1,3-thiazol-5-yl]-5-{[3-(trifluoromethyl)benzoyl]amino}benzamide

To a 10 mL round bottom flask charged with a magnetic stir bar and 2-chloro-N-(2-formyl-1,3-thiazol-5-yl)-5-{[3(trifluoromethyl)benzoyl]amino}benzamide (0.071 g, 0.16 mmol) (Example 86) was added anhydrous THF (2.5 mL) and the reaction was cooled to 0° C. A 3M solution of methyl magnesium bromide in Et₂O (0.15 mL, 0.468 mmol) was added to the reaction via syringe and the resulting mixture was allowed to stir at 0° C. for 0.5 h before being diluted with a saturated aqueous solution of NH₄Cl (20 mL). The mixture was then poured into a separatory funnel and extracted with EtOAc (50 mL). The organic phase was separated, dried with MgSO₄, filtered, and conc. in vacuo to yield the crude product. The crude oil was purified on SiO₂ (40 g) using EtOAc as eluent which afforded the title compound as a white solid; DMSO-D6 11.82 (s, 1H) 10.73 (s, 1H) 8.24-8.34 (m, 2H) 7.93-8.03 (m, 3H) 7.80 (t, 1H) 7.60 (d, 1H) 7.47 (s, 1H) 6.00 (s, 1H) 4.87 (q, 1H) 1.43 (d, 3H); m/z: 470.

### Example 84

### tert-Butyl (4-methyl-3-{[(2-methyl-1,3-thiazol-5-yl)amino]carbonyl}phenyl)carbamate

To a solution of 5-[(tert-butoxycarbonyl)amino]-2-methylbenzoic acid (Method 27, 2.9 g, 11.5 mmol) and 2-methyl-1,3-thiazol-5-amine (Method 2, 1.3 g, 11.4 mmol) in anhydrous DMF (10 ml) was added HATU (4.4 g, 11.6 mmol) and pyridine (4.6 ml, 56.9 mmol, 5 eq.). After stirring for 16 hours, the reaction mixture was diluted with EtOAc, washed with water, dried (Na₂SO₄) and concentrated. Purification by chromatography gave 3.4 g (85%) of a white solid. ¹H NMR 11.54 (s, 1H), 9.46 (s, 1H), 7.62 (s, 1H), 7.39-7.42 (m, 2H), 7.18 (d, 1H), 2.56 (s, 3H), 2.29 (s, 3H) 1.47 (s, 9H); m/z: 348.

### Example 85

The following compound was prepared by a procedure analogous to that used in the preparation of Example 84, using 2-methyl-1,3-thiazol-5-amine and the appropriate starting material.

| Ex. | Compound | m/z | SM |
|---|---|---|---|
| 85 | tert-Butyl (4-chloro-3-{[(2- | 367 | 5-[(tert-Butoxycarbonyl)amino]- |
| | methyl-1,3-thiazol-5- | | 2-chlorobenzoic acid |
| | yl)amino]carbonyl}- | | (Method 28) |
| | phenyl)carbamate | | |

### Example 86

### 2-Chloro-N-(2-formyl-1,3-thiazol-5-yl)-5-{[3(trifluoromethyl)benzoyl]amino}benzamide

To a 25 mL round bottom flask charged with a magnetic stir bar and 5-[(2-chloro-5-{[3-(trifluoromethyl)benzoyl]amino}benzoyl)amino]-N-methoxy-N-methyl-1,3-thiazole-2-carboxamide (0.28 g, 0.545 mmol) (Example 13) was added anhydrous THF (5 mL). The reaction was cooled to 0° C. and lithium aluminum hydride (0.03 g, 0.698 mmol) was added to the reaction mixture with stirring. The reaction mixture was stirred for 0.5 h before being carefully diluted with a saturated aqueous solution of NH₄Cl (˜20 mL). The mixture was then poured into a separatory funnel and extracted with EtOAc (˜50 mL). The organic phase was separated, dried with MgSO₄, filtered, and conc. in vacuo to yield the title compound as a colourless oil which was used without further purification; m/z: 454.

### Examples 87 and 88

The following compounds were prepared by a procedure analogous to that described in Example 86, using the appropriate starting material.

| Ex. | Compound | m/z | SM |
|---|---|---|---|
| 87 | 2-Chloro-5-[(3,5- | 414 | 5-({2-Chloro-5-[(3,5- |
| | dimethylbenzoyl)amino]- | | dimethylbenzoyl)amino]- |
| | N-(2-formyl-1,3- | | benzoyl}amino)-N- |
| | thiazol-5-yl)benzamide | | methoxy-N-methyl-1,3-thiazole- |
| | | | 2-carboxamide (Example 14) |
| 88 | 5-{[3-(1-Cyano-1- | 433 | 5-[(5-{[3-(1-Cyano-1- |
| | methylethyl)benzoyl]- | | methylethyl)benzoyl]amino}-2- |
| | amino}-N-(2-formyl- | | methylbenzoyl)amino]- |
| | 1,3-thiazol-5-yl)-2- | | N-methoxy-N-methyl-1,3- |
| | methylbenzamide | | thiazole-2-carboxamide |
| | | | (Example 15) |

What is claimed is:

1. A compound of formula (I): 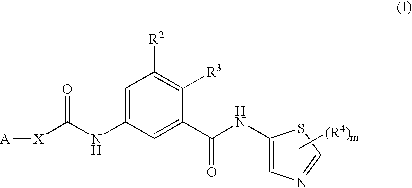
or a pharmaceutically acceptable salt thereof, wherein:
A is 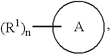
wherein Ring A is aryl, heteroaryl, carbocyclyl or heterocyclyl; wherein if said heteroaryl or heterocyclyl contains an —NH— moiety that nitrogen may be optionally substituted by a group selected from R⁵; or
A is C₁₋₆alkyl, C₂₋₆alkenyl, or C₂₋₆alkynyl; wherein A may be optionally substituted on carbon by one or more R^{8a}; and wherein if said heterocyclyl contains an —NH— moiety that nitrogen may be optionally substituted by a group selected from R^{9a};
R¹ is a substituent on carbon and is selected from halo, nitro, cyano, hydroxy, trifluoromethoxy, amino, carboxy, carbamoyl, mercapto, sulphamoyl, C₁₋₆alkyl, C₂₋₆alkenyl, C₂₋₆alkynyl, C₁₋₆alkoxy, C₁₋₆alkanoyl, C₁₋₆alkanoyloxy, N—(C₁₋₆alkyl)amino, N,N—(C₁₋₆alkyl)₂-amino, C₁₋₆alkanoylamino, N—(C₁₋₆alkyl)carbamoyl, N,N—(C₁₋₆alkyl)₂-carbamoyl, C₁₋₆alkylS(O)ₐ wherein a is 0 to 2, C₁₋₆alkoxycarbonyl, C₁₋₆alkoxycarbonylamino, N—(C₁₋₆alkyl)sulphamoyl, N,N—(C₁₋₆alkyl)₂sulphamoyl, N—(C₁₋₆alkyl)-N—(C₁₋₆alkoxy)sulphamoyl, N,N′—(C₁₋₆alkyl)₂ureido, N′,N′—(C₁₋₆alkyl)₂ureido, N—(C₁₋₆alkyl)-N′,N′—(C₁₋₆alkyl)₂ureido, C₁₋₁₆alkylsulphonylamino, carbocyclyl-R⁶— or heterocyclyl-R⁷—; wherein R¹ may be optionally substituted on carbon by one or more R⁸; and wherein if said heterocyclyl contains an —NH— moiety that nitrogen may be optionally substituted by a group selected from R⁹;
n is selected from 0-4; wherein the values of R¹ may be the same or different;
X is absent or is O or NRₐ, wherein Rₐ is H or C₁₋₆alkyl;
R² and R³ are independently selected from hydrogen, halo, nitro, cyano, hydroxy, trifluoromethoxy, amino, carboxy, carbamoyl, mercapto, sulphamoyl, C₁₋₆alkyl, C₂₋₆alkenyl, C₂₋₆alkynyl, C₁₋₆alkoxy, C₁₋₆alkanoyl, C₁₋₆alkanoyloxy, N—(C₁₋₆alkyl)amino, N,N—(C₁₋₆alkyl)₂-amino, C₁₋₆alkanoylamino, N—(C₁₋₆alkyl)carbamoyl, N,N—(C₁₋₆alkyl)₂-carbamoyl, C₁₋₆alkylS(O)ₐ wherein a is 0 to 2, C₁₋₆alkoxycarbonyl, N—(C₁₋₆alkyl)sulphamoyl, N,N—(C₁₋₆alkyl)₂sulphamoyl, C₁₋₆alkylsulphonylamino, carbocyclyl-R¹⁰— or heterocyclyl-R¹¹—; wherein R² may be optionally substituted on carbon by one or more R¹²; and wherein if said heterocyclyl contains an —NH— moiety that nitrogen may be optionally substituted by a group selected from R¹³;
R⁴ is selected from halo, cyano, amino, carboxy, carbamoyl, mercapto, sulphamoyl, ureido, C₁₋₆alkyl, C₂₋₆alkenyl, C₂₋₆alkynyl, C₁₋₆alkoxy, C₁₋₆alkanoyl, C₁₋₁₆alkanoyloxy, N—(C₁₋₁₆alkyl)amino, N,N—(C₁₋₆alkyl)₂-amino, C₁₋₁₆alkanoylamino, N—(C₁₋₆alkyl)carbamoyl, N,N—(C₁₋₆alkyl)₂-carbamoyl, N—(C₁₋₆alkyl)-N—(C₁₋₆alkoxy)carbamoyl, C₁₋₆alkylS(O)ₐ wherein a is 0 to 2, C₁₋₆alkoxycarbonyl, N—(C₁₋₆alkyl)sulphamoyl, N,N—(C₁₋₆alkyl)₂sulphamoyl, C₁₋₆alkylsulphonylamino, carbocyclyl-R¹⁴— or heterocyclyl-R¹⁵—; wherein R⁴ may be optionally substituted on carbon by one or more R¹⁶; and wherein if said heterocyclyl contains an —NH— moiety that nitrogen may be optionally substituted by a group selected from R¹⁷;
m is selected from 0-2; wherein the values of R⁴ may be the same or different;
R⁸, R^{8a}, and R¹² in each occurrence are independently selected from aryl, heteroaryl, halo, nitro, cyano, hydroxy, trifluoromethoxy, amino, carboxy, carbamoyl, mercapto, sulphamoyl, C₁₋₆alkyl, C₂₋₆alkenyl, C₂₋₆alkynyl, C₁₋₆alkoxy, C₁₋₆alkanoyl, C₁₋₆alkanoyloxy, N—(C₁₋₆alkyl)amino, N,N—(C₁₋₆alkyl)₂-amino, N—(C₁₋₆alkyl)-N—(C₁₋₆alkoxy)amino, C₁₋₆alkanoylamino, N—(C₁₋₆alkyl)carbamoyl, N,N—(C₁₋₆alkyl)₂-carbamoyl, C₁₋₆alkylS(O)ₐ wherein a is 0 to 2, C₁₋₆alkoxycarbonyl, C₁₋₆alkoxycarbonylamino, N—(C₁₋₆alkyl)sulphamoyl, N—(C₁₋₆alkyl)-N—(C₁₋₆alkoxy)sulphamoyl, N,N—(C₁₋₆alkyl)₂sulphamoyl, N,N′—(C₁₋₆alkyl)₂ureido, N′,N′—(C₁₋₆alkyl)₂ureido, N—(C₁₋₁₆alkyl)-N′,N′—(C₁₋₁₆alkyl)₂ureido, C₁₋₁₆alkylsulphonylamino, carbocyclyl-R¹⁸— or heterocyclyl-R¹⁹—; wherein R⁸ and R¹² independently of each other may be optionally substituted on carbon by one or more R²⁰; and wherein if said heterocyclyl contains an —NH— moiety that nitrogen may be optionally substituted by a group selected from R²¹;
R¹⁶ in each occurrence is independently selected from halo, nitro, cyano, hydroxy, trifluoromethoxy, amino, carboxy, carbamoyl, mercapto, sulphamoyl, C₁₋₆alkyl, C₂₋₆alkenyl, C₂₋₆alkynyl, C₁₋₆alkoxy, C₁₋₆alkanoyl, C₁₋₆alkanoyloxy, N—(C₁₋₆alkyl)amino, N,N—(C₁₋₆alkyl)₂-amino, C₁₋₆alkanoylamino, N—(C₁₋₆alkyl)carbamoyl, N,N—(C₁₋₆alkyl)₂-carbamoyl, C₁₋₆alkylS(O)ₐ wherein a is 0 to 2, C₁₋₆alkoxycarbonyl, N—(C₁₋₆alkyl)sulphamoyl, N,N—(C₁₋₆alkyl)₂sulphamoyl, C₁₋₆alkylsulphonylamino, carbocyclyl-R²²— or heterocyclyl-R²³—; wherein R may be optionally substituted on carbon by one or more R²⁴; and wherein if said heterocyclyl contains an —NH— moiety that nitrogen may be optionally substituted by a group selected from R²⁵;
R⁶, R⁷, R¹⁰, R¹¹, R¹⁴, R¹⁵, R¹⁸, R¹⁹, R²² and R²³ in each occurrence are independently selected from a direct bond, —O—, —N(R²⁶)—, —C(O)—, —N(R²⁷)C(O)—, —C(O)N(R²)—, —S(O)ₛ—, —SO₂N(R²⁹)— or —N(R³⁰)SO₂—; wherein R²⁶, R²⁷, R²⁸, R²⁹ and R³⁰ are independently selected from hydrogen or C₁₋₆alkyl and s is 0-2;
R⁵, R⁹, R^{9a} R¹³, R¹⁷, R²¹ and R²⁵ in each occurrence are independently selected from C₁₋₆alkyl, C₁₋₆alkanoyl, C₁₋₆alkylsulphonyl, C₁₋₆alkoxycarbonyl, carbamoyl, N—(C₁₋₆alkyl)carbamoyl, N,N—(C₁₋₆alkyl)carbamoyl, benzyl, benzyloxycarbonyl, benzoyl and phenylsulphonyl;
R²⁰ and R²⁴ in each occurrence are independently selected from halo, nitro, cyano, hydroxy, trifluoromethoxy, trifluoromethyl, amino, carboxy, carbamoyl, mercapto, sulphamoyl, methyl, ethyl, cyclopropyl, cyclobutyl, cyclopentyl, cyclohexyl, methoxy, ethoxy, acetyl, acetoxy, methylamino, ethylamino, dimethylamino, diethylamino, N-methyl-N-ethylamino, acetylamino, N-methylcarbamoyl, N-ethylcarbamoyl, N,N-dimethylcarbamoyl, N,N-diethylcarbamoyl, N-methyl-N-ethylcarbamoyl, phenyl, methylthio, ethylthio, methylsulphinyl, ethylsulphinyl, mesyl, ethylsulphonyl, methoxycarbonyl, ethoxycarbonyl, N-methylsulphamoyl, N-ethylsulphamoyl, N,N-dimethylsulphamoyl, N,N-diethylsulphamoyl or N-methyl-N-ethylsulphamoyl; wherein R²⁰ and R²⁴ may be optionally substituted on carbon by one or more R⁵⁰; and
R⁵⁰ in each occurrence is independently selected from halo, hydroxy, cyano, and C₁₋₆alkoxy.

2. A compound of formula (I), or a pharmaceutically acceptable salt thereof, as claimed in claim 1, wherein:
A is 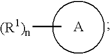
wherein Ring A is selected from aryl, heteroaryl, and carbocyclyl; wherein if said heteroaryl contains an —NH— moiety that nitrogen may be optionally substituted by a group selected from R⁵; or
A is C₁₋₆alkyl; wherein A may be optionally substituted on carbon by one or more R^{8a};
R⁵ is C₁₋₆alkyl;
R^{8a} in each occurrence is independently selected from halo, C₁₋₆alkoxy, and carbocyclyl-R¹⁸—, wherein R^{8a} may be optionally substituted on carbon by one or more R²⁰;
R¹⁸ is a direct bond; and
R²⁰ is methyl.

3. A compound of formula (I), or a pharmaceutically acceptable salt thereof, as in claim 1 wherein X is absent or O.

4. A compound of formula (I), or a pharmaceutically acceptable salt thereof, as in claim 1, wherein
R¹ is a substituent on carbon and is selected from halo, C₁₋₆alkyl, and carbocyclyl-R⁶—; wherein R¹ may be optionally substituted on carbon by one or more R⁸;
R⁶ is a direct bond; and
R⁸ in each occurrence is independently selected from halo and cyano.

5. A compound of formula (I), or a pharmaceutically acceptable salt thereof, as in claim 1, wherein R² and R³ are independently selected from hydrogen, halo, C₁₋₆alkyl.

6. A compound of formula (I), or a pharmaceutically acceptable salt thereof, as in claim 1, wherein:
R⁴ is selected from C₁₋₆alkyl, N—(C₁₋₆alkyl)carbamoyl, N—(C₁₋₆alkyl)-N—(C₁₋₆alkoxy)carbamoyl, carbocyclyl-R¹⁴— or heterocyclyl-R¹⁵—; wherein R⁴ may be optionally substituted on carbon by one or more R¹⁶;
R¹⁴ is a direct bond;
R¹⁵ is —C(O)—;
R¹⁶ in each occurrence is independently selected from hydroxy, N—(C₁₋₆alkyl)amino, N,N—(C₁₋₆alkyl)₂-amino, carbocyclyl-R²²— and heterocyclyl-R²³—; wherein R¹⁶ may be optionally substituted on carbon by one or more R²⁴; and wherein if said heterocyclyl contains an —NH— moiety that nitrogen may be optionally substituted by a group selected from R²⁵;
R²² is —N(R²⁶)—;
R²³ is a direct bond;
R²⁴ in each occurrence is independently selected from methyl, methoxy, dimethylamino, and cyclopropyl, wherein R²⁴ may be optionally substituted on carbon by one or more R⁵⁰;
R²⁵ is C₁₋₆alkyl;
R²⁶ is hydrogen; and
R⁵⁰ is hydroxy.

7. A compound of formula (I), or a pharmaceutically acceptable salt thereof, as in claim 1, wherein m is selected from 0 and 1.

8. A compound of formula (I), or a pharmaceutically acceptable salt thereof, as in claim 1, wherein n is selected from 0 to 2, wherein the values of R¹ may be the same or different.

9. A compound of formula (I): 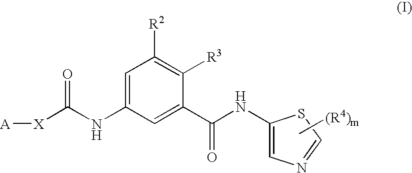
or a pharmaceutically acceptable salt thereof, wherein
A is selected from 3-(1-cyano-1-methylethyl)phenyl, 3-(trifluoromethyl)phenyl, 3-chlorophenyl, 3,5-dimethylphenyl, 3-fluoro-5-(trifluoromethyl)phenyl, 3-chloro-5-fluorophenyl, 3-cyclopropyl-5-fluorophenyl, 3,4-dichlorophenyl, 3-cyclopropylphenyl, 3-methylphenyl, 3-methylcyclohexyl, 2,6-dichloropyridin-4-yl, cyclopentyl, 3,4-dimethylphenyl, 6-methylpyridin-2-yl, 3-chloropyridin-4-yl, 5-methylpyridin-3-yl, 1,5-dimethyl-1H-pyrazol-3-yl, 5-methyl-1H-pyrazol-3-yl, 4-methylcyclohexyl, 3-(trifluoromethyl)cyclohexyl, 4,4-difluorocyclohexyl, 1-tert-butyl-5-methyl-1H-pyrazol-3-yl, 1-isopropyl-1H-pyrazol-3-yl, butyl, 3-methylpentyl, 2-methylbutyl, 3-methylbutyl, sec-butoxymethyl, cyclohexylmethyl, 2-methylprop-2-yl, (4-methylcyclohexyl)methyl, but-2-yl, hex-2-yl, cyclopropylmethyl, cyclopentylmethyl, and cyclohexyl(difluoro)methyl;
X is absent or O;
R¹ is a substituent on carbon and is selected from fluoro, chloro, methyl, trifluoromethyl, 2-cyanoprop-2-yl, and cyclopropyl;
R² is hydrogen;
R³ is selected from chloro and methyl;
R⁴ is selected from methyl, isopropyl, N-methylcarbamoyl, (4-methylpiperazin-1-yl)methyl, morpholincarbonyl, N-methyl-N-methoxycarbamoyl, hydroxymethyl, (dimethylamino)methyl, 1-hydroxyethyl, piperidinomethyl, (methylamino)methyl, morpholin-4-ylmethyl, 2-(dimethylamino)ethyl, 1-azetidinylmethyl, (cyclobutylamino)methyl, [(cyclopropylmethyl)amino]methyl, [(2-methoxyethyl)methylamino]methyl, [4-(hydroxymethyl)piperidin-1-yl]methyl, isopropyl, (cyclopropylamino)methyl, and cyclopropyl;
m is selected from 0 and 1; and
n is selected from 0 to 2, wherein the values of R¹ may be the same or different.

10. A compound of formula (I), selected from the group consisting of:
5-{[3-(1-Cyano-1-methylethyl)benzoyl]amino}-2-methyl-N-(2-methyl-1,3-thiazol-5-yl)benzamide;
2-Chloro-N-1,3-thiazol-5-yl-5-{[3-(trifluoromethyl)benzoyl]amino}benzamide;
2-Chloro-5-[(3-chlorobenzoyl)amino]-N-1,3-thiazol-5-ylbenzamide;
2-Chloro-5-[(3,5-dimethylbenzoyl)amino]-N-1,3-thiazol-5-ylbenzamide;
5-{[3-(1-Cyano-1-methylethyl)benzoyl]amino}-2-methyl-N-1,3-thiazol-5-ylbenzamide;
2-Methyl-N-(2-methyl-1,3-thiazol-5-yl)-5-{[3-(trifluoromethyl)benzoyl]amino}benzamide;
2-Chloro-5-[(3-chlorobenzoyl)amino]-N-(2-methyl-1,3-thiazol-5-yl)benzamide;
2-Chloro-5-[(3,5-dimethylbenzoyl)amino]-N-(2-methyl-1,3-thiazol-5-yl)benzamide;
2-Chloro-N-(2-methyl-1,3-thiazol-5-yl)-5-{[3-(trifluoromethyl)benzoyl]amino}benzamide;
2-Chloro-5-{[3-fluoro-5-(trifluoromethyl)benzoyl]amino}-N-(2-methyl-1,3-thiazol-5-yl)benzamide;
5-[(5-{[3-(1-Cyano-1-methylethyl)benzoyl]amino}-2-methylbenzoyl)amino]-N-methyl-1,3-thiazole-2-carboxamide;
5-{[3-fluoro-5-(trifluoromethyl)benzoyl]amino}-2-methyl-N-(2-methyl-1,3-thiazol-5-yl)benzamide;
5-[(3-Chloro-5-fluorobenzoyl)amino]-2-methyl-N-(2-methyl-1,3-thiazol-5-yl)benzamide;
5-[(3-Cyclopropyl-5-fluorobenzoyl)amino]-2-methyl-N-(2-methyl-1,3-thiazol-5-yl)benzamide;
5-[(3-Chlorobenzoyl)amino]-2-methyl-N-(2-methyl-1,3-thiazol-5-yl)benzamide;
5-[3,4-Dichlorobenzoyl)amino]-2-methyl-N-(2-methyl-1,3-thiazol-5-yl)benzamide;
5-[(3-Cyclopropylbenzoyl)amino]-2-methyl-N-(2-methyl-1,3-thiazol-5-yl)benzamide;
5-[(3,5-Dimethylbenzoyl)amino]-2-methyl-N-(2-methyl-1,3-thiazol-5-yl)benzamide;
2-methyl-5-[(3-methylbenzoyl)amino]-N-(2-methyl-1,3-thiazol-5-yl)benzamide;
2,6-Dichloro-N-(4-methyl-3-{[(2-methyl-1,3-thiazol-5-yl)amino]carbonyl}phenyl)isonicotinamide;
2-Methyl-5-{[(3-methylcyclohexyl)carbonyl]amino}-N-(2-methyl-1,3-thiazol-5-yl)benzamide;
2-Methyl-N-(2-methyl-1,3-thiazol-5-yl)-5-(pentanoylamino)benzamide; and
2-methyl-5-[(4-methylhexanoyl)amino]-N-(2-methyl-1,3-thiazol-5-yl)benzamide.

11. (canceled)

12. (canceled)

13. (canceled)

14. (canceled)

15. (canceled)

16. A method for producing a CSF-1R kinase inhibitory effect in a warm-blooded animal, such as man, in need of such treatment, said method comprising administering to said animal an effective amount of a compound of formula (I), or a pharmaceutically salt thereof, as in claim 1.

17. A method for producing an anti-cancer effect in a warm-blooded animal such as man, in need of such treatment, said method comprising administering to said animal an effective amount of a compound of formula (I), or a pharmaceutically acceptable salt thereof, as in claim 1.

18. A method for treating breast, ovarian, bladder, cervical, endometrial, prostate, lung, kidney and pancreatic tumors; haematological malignancies including myelodysplastic syndrome, acute myelogenous leukemia, chronic myelogenous leukemia, non Hodgkin's lymphoma, Hodgkin's disease, multiple myeloma and chronic lymphocytic leukemia; and glioma, squamous cell carcinoma of the esophagus, malignant uveal melanoma and follicular lymphoma, in a warm-blooded animal, such as man, in need of such treatment, said method comprising administering to said animal an effective amount of a compound of formula (I), or a pharmaceutically acceptable salt thereof, as in claim 1.

19. A method for treating tumor-associated osteolysis, osteoporosis including ovariectomy-induced bone loss, orthopedic implant failure, autoimmune disorders including systemic lupus erythematosus, arthritis including rheumatoid arthritis, osteoarthritis, renal inflammation and glomerulonephritis; inflammatory bowel disease; transplant rejection including renal and bone marrow allografts and skin xenograft, atherosclerosis, obesity, Alzheimer's Disease and Langerhans cell histiocytosis, in a warm blooded animal, such as man, in need of such treatment, said method comprising administering to said animal an effective amount of a compound of formula (I), or a pharmaceutically salt thereof, as in claim 1.

20. A pharmaceutical composition comprising a compound of formula (I), or a pharmaceutically acceptable salt thereof, as in claim 1, and at least one pharmaceutically acceptable carrier, diluent, or excipient.

21. (canceled)

22. (canceled)

23. (canceled)

24. (canceled)

25. (canceled)

26. (canceled)

27. (canceled)

28. (canceled)
